(19) United States
(12) Patent Application Publication
(10) Pub. No.: US 2022/0323532 A1
(43) Pub. Date: Oct. 13, 2022

(54) COMPOSITIONS COMPRISING PLANT-DERIVED EXOSOME-LIKE NANOVESICLES OR EXOSOMES AND METHODS OF USE THEREOF

(71) Applicant: Nutraceutical Wellness Inc., New York, NY (US)

(72) Inventors: Giorgio DELL'ACQUA, Jersey City, NJ (US); Aleksander RICHARDS, New York, NY (US); Rebecca SCOTLAND, Miami Beach, FL (US); Roland PERALTA, Monroe, NJ (US)

(21) Appl. No.: 17/710,929

(22) Filed: Mar. 31, 2022

Related U.S. Application Data
(60) Provisional application No. 63/168,936, filed on Mar. 31, 2021.
(60) Provisional application No. 63/237,064, filed on Aug. 25, 2021.

(51) Int. Cl. A61K 36/81; A61K 9/127; A61K 31/4045; A61K 35/747; A61K 31/047; A61K 31/455; A61K 31/728; A61K 47/10; A61K 31/522; A61K 47/38; A61K 36/03; A61K 36/04; A61K 47/24; A61K 36/31; A61K 36/185; A61K 36/48; A61K 36/258; A61P 17/14

(52) U.S. Cl. CPC A61K 36/81; A61K 9/127; A61K 31/4045; A61K 35/747; A61K 31/047; A61K 31/455; A61K 31/728; A61K 47/10; A61K 31/522; A61K 47/38; A61K 36/03; A61K 36/04; A61K 47/24; A61K 36/31; A61K 36/185; A61K 36/48; A61K 36/258; A61P 17/14; A61K 2236/31; A61K 2236/37

(57) ABSTRACT

The present disclosure provides a composition comprising (a) exosome-like nanovesicles or exosomes and (b) a carrier, wherein the exosome-like nanovesicles or exosomes are extracted from Withania somnifera and methods of effecting a change in hair appearance, hair growth, hair pigmentation, hair follicle size or hair shaft size, comprising administering to the skin of a subject in need thereof an effective amount of such a composition.

### CROSS-REFERENCE TO RELATED APPLICATIONS

This application claims the benefit of U.S. Provisional Application No. 63/168,936, filed on Mar. 31, 2021, and 63/237,064, filed on Aug. 25, 2021, each of which is incorporated by reference herein in its entirety.

### BACKGROUND

Hair loss (alopecia) is a widespread problem affecting about 80 million men and women in the United States alone according to the American Academy of Dermatology. The $7 billion hair loss industry is a testament to the significance and the scope of the issue. The most common alopecias are androgenic alopecia, telogen effluvium and alopecia areata. Accordingly, there is a need for improved compositions or methods for treating alopecia.

### SUMMARY OF THE INVENTION

In one aspect, the present invention provides compositions comprising (a) exosome-like nanovesicles or exosomes and (b) a carrier, wherein the exosome-like nanovesicles or exosomes are extracted from Withania somnifera.

In another aspect, the present invention provides methods of promoting hair growth or reducing hair loss, comprising administering to a subject in need thereof an effective amount of a composition as described in any of the embodiments herein for a time sufficient to promote hair growth or reduce hair loss.

In another aspect, the present invention provides methods of preventing, reducing or reversing hair loss, comprising administering to a subject in need thereof an effective amount of a composition as described in any of the embodiments herein for a time sufficient to prevent, reduce or reverse hair loss.

In another aspect, the present invention provides methods for effecting a change in mammalian hair appearance, hair growth, hair pigmentation, hair follicle size or hair shaft size, comprising administering to the skin of a mammal in need thereof an effective amount of a composition as described in any of the embodiments herein for a time sufficient to effect a change in mammalian hair appearance, hair growth, hair pigmentation, hair follicle size or hair shaft size.

In another aspect, the present invention provides methods for producing a melanogenetic action in hair or promoting its pigmentation, comprising administering to a subject in need thereof an effective amount of a composition as described in any of the embodiments herein for a time sufficient to produce a melanogenetic action in the hair or promote its pigmentation.

In another aspect, the present invention provides methods of stimulating hair growth or preventing hair loss, comprising topically administering to a subject in need thereof an effective amount of a composition comprising (a) Withania somnifera-extracted exosome-like nanovesicles or exosomes and (b) a carrier, wherein:

the amount of the amount of the Withania somnifera-extracted exosome-like nanovesicles or exosomes is from about 0.1% to about 5% by weight of the composition,

the number of Withania somnifera-extracted exosome-like nanovesicles or exosomes is from about 1×10⁸ per mL of the composition to about 1×10¹⁰ per mL of the composition, and

the Withania somnifera is dried Withania somnifera seeds.

In another aspect, the present invention provides methods of promoting hair growth or reducing hair loss, comprising administering to dermal papilla of a subject in need thereof an effective amount of a composition comprising Withania somnifera-extracted exosome-like nanovesicles or exosomes having an increased level of heat shock stress-response exosomes,

wherein the Withania somnifera is a stem, root, leaf, or fruit of a Withania somnifera plant, wherein the Withania somnifera plant is grows at a conditioning temperature

In another aspect, the present invention provides kits for promoting hair growth or preventing, reducing, or reversing hair loss, comprising a composition as described in any of the embodiments herein and instructions for topically administering the composition to a scalp of a subject in need of hair-growth promotion or prevention, reduction or reversal of hair loss.

In another aspect, the present invention provides uses of a composition as described in any of the embodiments herein for promoting hair growth or preventing, reducing, or reversing hair loss in a subject in need thereof.

### BRIEF DESCRIPTION OF THE DRAWINGS

The accompanying drawings, which are included to provide a further understanding of the present disclosure, are incorporated in and constitute a part of this specification, illustrate aspects of the present disclosure and, together with the detailed description, serve to explain the principles of the present disclosure. The patent or application file contains at least one drawing executed in color. Copies of this patent or patent application publication with color drawing(s) will be provided by the Office upon request and payment of the necessary fee.

FIG. 1 shows an example of the Ashwagandha exosome protein analysis by Western Blot.

FIG. 2 shows an example of the Ashwagandha exosome protein analysis by immunoprecipitation followed by Western Blot.

FIG. 3 shows the regions from the Ashwagandha plant where exosomes of the present disclosure are harvested. Specifically, the Ashwagandha root is represented by the number 1, the Ashwagandha central stem is represented by the number 2, the Ashwagandha leaf stem is represented by the number 3, the Ashwagandha leaf is represented by the number 4, the Ashwagandha fruit is represented by the number 5, and the Ashwagandha seed is represented by the number 6.

FIG. 4A depicts dermal fibroblast treated with a P. acnes lysate to induce oxidative stress. Viability for both Ashwagandha Seed Derived Exosomes (ASHWG1) and Human Adipo Tissue Mesenchimal Stem Cells Derived Exosomes (MSC ZEN) as a control was not different than Media Only Negative Control in the presence of P. acnes lysate, suggesting a lack of toxicity by the exosomes after 6 hours.

FIG. 4B. depicts dermal fibroblast treated with a P. acnes lysate to induce oxidative stress. Viability for both Ashwagandha Seed Derived Exosomes (ASHWG1) and Human Adipo Tissue Mesenchimal Stem Cells Derived Exosomes (MSC ZEN) as a control was not different than Media Only Negative Control in the presence of P. acnes lysate, suggesting a lack of toxicity by the exosomes after 24 hours.

FIG. 5A shows the P. acnes triggered Super Oxide release in Dermal Fibroblasts at 6 h. Both Ashwagandha Seed Derived Exosomes (ASHWG1) and Human Adipo Tissue Mesenchimal Stem Cells Derived Exosomes (MSC ZEN) were not different than Media Only Negative Control in the presence of P. acnes lysate and induction of oxidative stress, suggesting a lack of toxicity by the exosomes.

FIG. 5B shows the P. acnes triggered Super Oxide release in Dermal Fibroblasts at 24 h. Both Ashwagandha Seed Derived Exosomes (ASHWG1) and Human Adipo Tissue Mesenchimal Stem Cells Derived Exosomes (MSC ZEN) were not different than Media Only Negative Control in the presence of P. acnes lysate and induction of oxidative stress, suggesting a lack of toxicity by the exosomes. Interestingly ASHWG1 (1×10¹⁰ and 1×10⁹) seemed to reduce the release of Super Oxide.

FIG. 6A. shows the P. acnes triggered ROS release in Dermal Fibroblasts. Both Ashwagandha Seed Derived Exosomes (ASHWG1) and Human Adipo Tissue Mesenchimal Stem Cells Derived Exosomes (MSC ZEN) reduced P. acnes induced ROS release.

FIG. 6B shows the P. acnes triggered ROS release in Dermal Fibroblasts. Both Ashwagandha Seed Derived Exosomes (ASHWG1) and Human Adipo Tissue Mesenchimal Stem Cells Derived Exosomes (MSC ZEN) showed no difference compared to control (Media only).

FIG. 7 shows a serum free migration assay in human entothelial cells (UVEC). 1×10⁹ NV/mL of Ashwagandha Seed Derived Exosome (ASHWG) treatment shows significant increase in cell migration above Serum free (SF) control and 1×10⁹ adipose Human Adipose Tissue Mesenchymal Stem Cells Derived Exosomes (MSC). 1×10¹⁰ ASHWG and 1×10¹⁰ MSC exosomes also demonstrated enhanced cell migration above that of the control media.

FIG. 8 shows Ashwagandha Derived Seed Exosomes treated human dermal fibroblasts stimulating cells migration compared to serum free, growth factor-free base media alone (SF). This increase is most evident at 16 and 24 h incubation. Complete: Complete media. Error bars are shown as ±SEM. 2way ANOVA statistical test shows significance for 1×10⁹ and 1×10¹⁰ at 16 h and 24 h incubation compared to SF.

FIG. 9 shows Ashwagandha Derived Seed Exosomes treated human dermal fibroblasts, particularly at 1×10⁸ and 1×10⁹ concentrations, stimulating cells migration compared to serum 1/20 media alone ( 1/20). This increase is most evident at 24 h incubation. Complete: Complete media. Error bars are shown as ±SEM. 2way ANOVA statistical test shows significance for 1×10⁹ at 24 hours incubation compared to 1/20.

FIG. 10 shows the data from Ashwagandha Exosomes treated Human Hair Follicle Dermal Papilla Cells. At 1×10¹⁰ concentration, an increase in growth both at baseline and in the presence of growth inhibitor Cortisol was detected. This increase was significant for Ashwagandha Seed and Stem derived Exosomes. *p<0.05 vs CT (untreated control), Student's T test.

FIG. 11A depicts Inflammatory Marker IL-29 Expression in Dermal Fibroblasts Treated with a P. acnes Lysate. Time point is 6 hours after treatment. CT: Untreated, ASH: Ashwagandha Seed Derived Exosomes, MSC: Adipose Tissue Derived Stem Cells Exosomes, Dex: Dexamethasone, **p<0.01, *p<0.05 vs CT (+P. acnes lysate), Student's t Test.

FIG. 11B depicts Inflammatory Marker IL-10 Expression in Dermal Fibroblasts Treated with a P. acnes Lysate. Time point is 6 hours after treatment. CT: Untreated, ASH: Ashwagandha Seed Derived Exosomes, MSC: Adipose Tissue Derived Stem Cells Exosomes, Dex: Dexamethasone, **p<0.01, *p<0.05 vs CT (+P. acnes lysate), Student's t Test.

FIG. 11C depicts Inflammatory Marker IL-4 Expression in Dermal Fibroblasts Treated with a P. acnes Lysate. Time point is 6 hours after treatment. CT: Untreated, ASH: Ashwagandha Seed Derived Exosomes, MSC: Adipose Tissue Derived Stem Cells Exosomes, Dex: Dexamethasone, **p<0.01, *p<0.05 vs CT (+P. acnes lysate), Student's t Test.

FIG. 11D depicts Inflammatory Marker EGF Expression in Dermal Fibroblasts Treated with a P. acnes Lysate. Time point is 6 hours after treatment. CT: Untreated, ASH: Ashwagandha Seed Derived Exosomes, MSC: Adipose Tissue Derived Stem Cells Exosomes, Dex: Dexamethasone, **p<0.01, *p<0.05 vs CT (+P. acnes lysate), Student's t Test.

FIG. 12 shows the uptake of Ashwagandha exosomes by Human Dermal Fibroblasts. Exosome: green staining; Dermal Fibroblasts Nuclei: blue staining.

FIG. 13 shows zoomed images of dermal fibroblasts after 16 hours of treatment with labeled Ashwagandha exosomes. Exosomes appear in cytoplasmic compartments at various intensities. At 4 C, the exosomes do not show internalization into the cell and appear to be dispersed in the media in clumps.

FIG. 14 shows progressive uptake of Ashwagandha exosomes by human dermal fibroblasts in a dose-dependent and time-dependent pattern at 37 C (solid lines), while the dotted lines represent uptake at 4 C.

FIG. 15 shows Ashwagandha dry seed EVs promoting HUVEC tubulogenesis at 1×10⁹ particle/mL dosage. Data represented of mean±SD. Numbers below plant type EVs represent total particles added per well for each change in treatment media. One-way ANOVA Dunnett post hoc test (*p<0.05, **p<0.01, ***p<0.001, ****p<0.0001). n=3 biological replicates. Total Tube Area: Total tube area (excluding nodes) in μm2. Total Tube Length: Total length of the tubes in μm; Segments: Number of tube segments connecting branch points and/or ends; Branch Points: Number of junctions connecting segments (excluding nodes, which are not considered branches); Connected Sets: Number of distinct objects detected in the image not connected to one another (no path of connected pixels of tubes or nodes connects the objects). Measures the overall connectivity of the growth network (a completely connected network would have just one connected set of pixels); Mean Tube Length: Total tube length divided by the number of segments; Mean Tube Area: Mean Tubule Area Total tube area divided by the number of segments; Tube Length Per Set: Total tube length in microns divided by the number of connected sets.

FIG. 16 shows Ashwagandha Seed Derived Exosomes (ASH) compared to Aloe Leaf Derived Exosomes to induce HUVEC tubule formation. ASH at 1×10⁹ was superior to Aloe Exosomes at the same concentration.

FIG. 17 shows the positive Control VEGF-A to induce HUVEC tubule formation. ECGM-1: standard endothelial growth media.

FIG. 18 shows electron microscopy image of Ashwagandha Seed Derived Exosomes or ELN (Exosome Like Nanovescicles) FIG. 19 shows VEGF-A induction by Aloe Leaf Derived Exosomes or ELN (as a control) and Ashwagandha Seed Derived Exosomes or ELN by net concentration (top) and by fold change (bottom).

FIG. 20 shows Ashwagandha ELN (Evs) increasing melanogenesis in B16 melanoma cells after 72 h treatment. B16 cells were triggered to produce melanin with α-MSH. Kojic Acid was used as an inhibitor of melanogenesis.

FIG. 21 shows the procedure for extracting the Ashwagandha derived exosomes or ELN from the Ashwagandha seeds along with the corresponding yield and concentration.

FIG. 22 shows the penetration studies according to embodiments of the present disclosure.

FIG. 23 shows the results from an ORAC assay indicating antioxidant activity related to the Ashwagandha exosomes or ELNs.

FIG. 24A-C show the overall safety and perception profile for the Ashwagandha exosomes or ELNs in A) a primary skin irritation evaluation; B) repeat insult patch test; and C) consumer perception and tolerability evaluation.

FIGS. 25A-D show a series of graphs depicting that Ashwagandha seed plant exosome-like nanovesicles (Ash-PLEN) stimulate in vitro human dermal hair follicle cell (hHFDPC) growth factor expression. FIG. 25A shows an increase in leukemia inhibitory factor (LIF) expression. FIG. 25B shows an increase in placental growth factor 1 (PLGF-1) expression. FIG. 25C shows an increase in basic fibroblast growth factor (FGF-2) expression. FIG. 25D shows an increase in vascular endothelial secreted growth factor A (VEGF-A) expression.

FIG. 26 shows the increase in melanin production in human primary melanocytes treated with Ashwagandha nanovesicles in a dose-dependent manner.

FIG. 27 shows treatment with Ashwagandha nanovesicles (exosomes) prolongs Anagen Phase in human hair follicles after 5 days in human dissected hair follicle ex vivo organ culture.

FIG. 28 shows the western blot characterization of ashwagandha seed derived nanovesicles (ASH-NV). Three ASH-NV Sample Lots were obtained. Samples were compared by loading an equal amount of protein per well (35 ug). Membranes were probed with plant antibodies directed at plant Hsp70, Hsp90, Actin, TET8 and with human antibodies directed at plant HSP70. The antibodies were all tested at 1:1000 dilution followed by washing and secondary antibody anti-rabbit HRP at 1:3000. Membranes were exposed to chemiluminescent reagent and imaged on BioRad Gel documentation system. Expected Band Size was Hsp70=˜70 KDa, TET8=˜31 KDa, Hsp90=˜90, Actin=˜40.

### DETAILED DESCRIPTION OF THE INVENTION

While the present disclosure is described herein with reference to illustrative embodiments for particular applications, it should be understood that embodiments of the present disclosure are not limited thereto. Other embodiments are possible, and modifications can be made to the described embodiments within the spirit and scope of the teachings herein, as they may be applied to the field of the present disclosure or to any additional fields in which such embodiments would be of significant utility.

In one aspect, the present disclosure is directed to a composition comprising (a) exosome-like nanovesicles or exosomes and (b) a carrier, wherein the exosome-like nanovesicles or exosomes are extracted from Withania somnifera.

In some embodiments, the composition is useful for stimulating hair growth or preventing hair loss in a subject.

In some embodiments, the Withania somnifera is Withania somnifera stem, Withania somnifera root, Withania somnifera leaf, Withania somnifera fruit, or Withania somnifera seed.

In some embodiments, the exosome-like nanovesicles or exosomes are extracted from Withania somnifera seed.

In some embodiments, the Withania somnifera is heat shocked Withania somnifera.

In some embodiments, the Withania somnifera is not heat shocked.

In some embodiments, the composition is useful for treating, preventing, or reversing sparse hair growth, short hair growth, thin hair growth, partial or complete hair loss on the scalp, alopecia, androgenic alopecia, alopecia androgenetica, male pattern baldness, female pattern baldness, non-androgenic alopecia, alopecia areata, alopecia totalis, alopecia universalis, radiation induced alopecia, alopecia due to radiotherapy, drug induced alopecia, alopecia due to chemotherapy, traumatic alopecia, scarring alopecia, psychogenic alopecia, stress related alopecia, cortisol related alopecia or anagen effluvium.

In some embodiments, the composition is a topical composition.

In some embodiments, the composition is a liquid, an ointment or a cream.

In some embodiments, the composition is a liquid.

In some embodiments, the composition is a cosmetic composition.

In some embodiments, the composition is useful for preventing or reversing cortisol induced growth arrest in human follicle dermal papilla cells.

In some embodiments, the Withania somnifera is dried.

In some embodiments, the Withania somnifera is dried Withania somnifera seeds.

In some embodiments, the Withania somnifera is freeze-dried.

In some embodiments, the Withania somnifera is freeze-dried Withania somnifera seeds.

In some embodiments, the composition of the present disclosure further comprises aloe-extracted exosome-like nanovesicles or aloe-extracted exosomes.

In some embodiments, the composition of the present disclosure further comprises human exosomes.

In some embodiments, the number of Withania somnifera-extracted exosome-like nanovesicles or exosomes is from about 1×10⁷ per mL of the composition to about 1×10¹² per mL of the composition.

In some embodiments, the number of extracted Withania somnifera-extracted exosome-like nanovesicles or exosomes is about 1×10⁷ per mL of the composition, about 1×10⁸ per mL of the composition, about 1×10⁹ per mL of the composition, about 1×10¹⁰ per mL of the composition, about 1×10¹¹ per mL of the composition, or about 1×10¹² per mL of the composition.

In some embodiments, the number of Withania somnifera-extracted exosome-like nanovesicles or exosomes is from about 1×10⁹ per mL of the composition to about 1×10¹⁰ per mL of the composition.

In some embodiments, the number of Withania somnifera-extracted exosome-like nanovesicles or exosomes is about 1×10⁹ per mL of the composition.

In some embodiments, the number of Withania somnifera-extracted exosome-like nanovesicles or exosomes is about 1×10¹⁰ per mL of the composition.

In some embodiments, the composition further comprises aloe-extracted exosome-like nanovesicles or exosomes, and the number of the aloe-extracted exosome-like nanovesicles or exosomes is from about 1×10⁷ per mL of the composition to about 1×10¹² per mL of the composition.

In some embodiments, the number of aloe-extracted exosome-like nanovesicles or exosomes is about 1×10⁷ per mL, about 1×10⁸ per mL, about 1×10⁹ per mL, about 1×10¹⁰ per mL, about 1×10¹¹ per mL, or about 1×10¹² per mL of the composition.

In some embodiments, number of aloe-extracted exosome-like nanovesicles or exosomes is from about 1×10⁹ per mL to about 1×10¹⁰ per mL.

In some embodiments, the number of aloe-extracted exosome-like nanovesicles or exosomes is about 1×10⁹ per mL of the composition.

In some embodiments, the number of aloe-extracted exosome-like nanovesicles or exosomes within the composition is about 1×10¹⁰ per mL of the composition.

In some embodiments, the Withania somnifera-extracted exosome-like nanovesicles or exosomes are purified.

In some embodiments, the carrier comprises an aqueous solution, suspension or mixture.

In some embodiments, the composition further comprises glycerin, Melaleuca alternifolia leaf water, propanediol, 1,2-hexanediol, panthenol, niacinamide, hydroxyethylcellulose, Lepidium meyenii root extract, maltodextrin, caprylhydroxamic acid, Hippophae rhamnoides fruit extract, Equisetum arvense extract, Laminaria saccharina extract, Chondrus crispus extract, sodium metabisulfite, alcohol, phospholipids, arginine, lactic acid, melatonin, potassium sorbate, lactobacillus ferment, Pisum sativum extract, or phosphate buffered saline.

In some embodiments, the composition further comprises glycerin, Camellia sinensis (green tea) leaf extract, glycine, Larix europaea wood extract, sodium metabisulfite, zinc chloride, Pisum sativum (pea) sprout extract, alcohol, Olea europaea (olive) leaf extract, Curcuma longa(turmeric) root extract, Equisetum arvense (horsetail) extract, Hippophae rhamnoides (sea buckthorn) fruit oil, Laminaria saccharina (neptune kelp) extract, Lepidium meyenii (maca) root extract, Melaleuca alternifolia (tea tree) leaf oil, Moringa oleifera (moringa) leaf extract, Panax ginseng (ginseng) root extract, DL-panthenol, L-theanine, Melatonin, Niacinamide, sodium dehydroacetate, sodium hyaluronate, or phytic acid.

In some embodiments, the composition further comprises water, glycerin, Melaleuca alternifolia leaf water, propanediol, butylene glycol, caffeine, 1,2-hexanediol, niacinamide, hydroxyethylcellulose, panthenol, Lepidium meyenii root extract, maltodextrin, caprylhydroxamic acid, Chondrus crispus extract, Hippophae rhamnoides fruit extract, Laminaria saccharina (neptune kelp) extract, an alcohol, phospholipids, sodium metabisulfite, arginine, lactic acid, melatonin, potassium sorbate, lactobacillus ferment, Pisum sativum extract, phosphate buffered saline, or Panax ginseng root extract.

In some embodiments, the carrier is water, and the composition further comprises glycerin, an aqueous buffer, or a naturally occurring preservative.

In some embodiments, the composition further comprises a naturally occurring preservative.

In some embodiments, the naturally occurring preservative comprises lactobacillus ferment.

In some embodiments, the composition further comprises melatonin.

In some embodiments, the composition further comprises niacinamide.

In some embodiments, the composition further comprises an alcohol.

In some embodiments, the alcohol is ethyl alcohol.

In some embodiments, the composition comprises the Withania somnifera-extracted exosome-like nanovesicles or exosomes in an amount of about 0.01% to 10% by weight of the composition.

In some embodiments, the composition comprises the Withania somnifera-extracted exosome-like nanovesicles or exosomes in an amount of about 0.1% to 5% by weight of the composition.

In some embodiments, the composition comprises the Withania somnifera-extracted exosome-like nanovesicles or exosomes in an amount of about 0.1% to 4% by weight of the composition.

In some embodiments, the composition comprises the Withania somnifera-extracted exosome-like nanovesicles or exosomes in an amount of about 0.1% to 3% by weight of the composition.

In some embodiments, the composition comprises the Withania somnifera-extracted exosome-like nanovesicles or exosomes in an amount of about 0.1% to 2% by weight of the composition.

In some embodiments, the composition comprises the Withania somnifera-extracted exosome-like nanovesicles or exosomes in an amount of about 0.1% to 1% by weight of the composition.

In some embodiments, the composition comprises the Withania somnifera-extracted exosome-like nanovesicles or exosomes in an amount of about 0.3% to about 1% by weight of the composition.

In another aspect, the present disclosure is directed to a method of promoting hair growth or reducing hair loss, comprising administering to a subject in need thereof an effective amount of a composition as described in any of the embodiments herein for a time sufficient to promote hair regrowth or reduce hair loss.

In another aspect, the present disclosure is directed to a method of preventing, reducing or reversing hair loss, comprising administering to a subject in need thereof an effective amount of a composition as described in any of the embodiments herein for a time sufficient to prevent, reduce or reverse hair loss.

In some embodiments, the hair loss is caused or mediated by cortisol or stress.

In another aspect, the present disclosure is directed to a method for effecting a change in mammalian hair appearance, hair growth, hair pigmentation, hair follicle size or hair shaft size, comprising administering to the skin of a mammal in need thereof an effective amount of a composition as described in any of the embodiments herein for a time sufficient to effect a change in mammalian hair appearance, hair growth, hair pigmentation, hair follicle size or hair shaft size.

In some embodiments, administering is topically administering.

In another aspect, the present disclosure is directed to a method for producing a melanogenetic action in hair or promoting its pigmentation, comprising administering to a subject in need thereof an effective amount of a composition as described in any of the embodiments herein for a time sufficient to produce a melanogenetic action in the hair or promote its pigmentation.

In another aspect, the present disclosure is directed to a method of stimulating hair growth or preventing hair loss, comprising topically administering to a subject in need thereof a composition comprising (a) Withania somnifera-extracted exosome-like nanovesicles or exosomes and (b) a carrier, wherein:

the amount of the Withania somnifera-extracted exosome-like nanovesicles or exosomes is from about 0.1% to about 5% by weight of the composition,

the number of Withania somnifera-extracted exosome-like nanovesicles or exosomes is from about 1×10⁸ per mL of the composition to about 1×10¹⁰ per mL of the composition, and

the Withania somnifera is dried Withania somnifera seeds.

In another aspect, the present disclosure is directed to a method of promoting hair growth or reducing hair loss comprising,

administering to dermal papilla of a subject in need thereof a composition comprising Withania somnifera-extracted exosome-like nanovesicles or exosomes having an increased level of heat shock stress-response exosomes,

wherein the Withania somnifera-extracted exosome-like nanovesicles or exosomes are extracted from Withania somnifera stem, Withania somnifera root, Withania somnifera leaf, or Withania somnifera fruit of a Withania somnifera plant, wherein the Withania somnifera plant is grown at a conditioning temperature.

In some embodiments, the conditioning temperature is about 33° C. to about 45° C.

In some embodiments, the Withania somnifera is grown at the conditioning temperature for about 1 hour to about 5 hours.

In some embodiments, the Withania somnifera plant is grown at a conditioning temperature of about 33° C. to about 45° C. for about 1 hour to about 5 hours.

In some embodiments, the conditioning temperature is about 45° C.

In some embodiments, the Withania somnifera plant is primed by warming it at a priming temperature prior to growing it at the conditioning temperature.

In some embodiments, the Withania somnifera plant is primed by warming it at a priming temperature prior to growing it at the conditioning temperature, wherein the priming temperature is about 20° C. to about 33° C.

In another aspect, the present disclosure is directed to a kit for promoting hair growth or preventing, reducing, or reversing hair loss, comprising a composition as described in any of the embodiments herein and instructions for topically administering the composition to a scalp of a subject in need of hair-growth promotion or hair-loss prevention, reduction or reversal.

In another aspect, the present disclosure directed to the use a composition as described in any of the embodiments herein for promoting hair growth or preventing, reducing, or reversing hair loss in a subject in need thereof.

In another aspect, the present disclosure directed to Withania somnifera-extracted exosome-like nanovesicles or exosomes for use in the preparation of a cosmetic composition as described in any of the embodiments herein for promoting hair growth or preventing, reducing, or reversing hair loss in a subject in need thereof.

The present disclosure is directed to plant exosome-like nanovesicles and/or plant-derived exosomes, formulations/compositions, and methods of use thereof. In some embodiments, the present disclosure is directed to a method of treating or a method of preventing hair loss using plant exosome-like nanovesicles and/or plant-derived exosomes or a formulation/composition thereof. In some embodiments, the present disclosure is directed to Withania somnifera-derived exosome-like nanovesicles and/or exosomes, formulations/compositions, and methods of use thereof. In some embodiments, the present disclosure is directed to method of treating or a method of preventing hair loss using a Withania somnifera-derived exosome-like nanovesicles and/or exosomes or a formulation/composition thereof.

Accordingly, the isolated Withania somnifera-derived exosome-like nanovesicles and/or exosomes produced according to the methods provided herein can have advantages over existing systemic or direct application of pharmaceuticals or plant extracts for promoting hair growth or preventing hair loss.

In some embodiments, the exosome-like nanovesicles and/or exosomes of the present disclosure are derived from Withania somnifera (Ashwagandha). In some embodiments, the plant source for the exosome-like nanovesicles and/or exosomes of the present disclosure is Withania somnifera. In some embodiments, the exosome-like nanovesicles and/or exosomes are derived from one or more of the Withania somnifera root, Withania somnifera stem, Withania somnifera leaf, Withania somnifera fruit, and/or Withania somnifera seed. In some embodiments, the exosome-like nanovesicles and/or exosomes are derived from one or more of the heat shocked Withania somnifera root, heat shocked Withania somnifera stem, heat shocked Withania somnifera leaf, heat shocked Withania somnifera fruit, and heat shocked Withania somnifera seed.

In some embodiments, the Withania somnifera-derived exosome-like nanovesicles and/or exosomes are within a composition. In some embodiments, the Withania somnifera exosome-like nanovesicles and/or exosomes are within a cosmetic composition. In some embodiments, the Withania somnifera exosome-like nanovesicles and/or exosomes are within a cosmetic composition and applied topically. In some embodiments, the Withania somnifera-derived exosome-like nanovesicles and/or exosomes are used for the treatment of various types of hair loss.

In some embodiments, the present disclosure provides for the use of a composition comprising Withania somnifera exosome-like nanovesicles and/or exosomes to promote or enhance hair growth. In some embodiments, the present disclosure provides for the use of Withania somnifera exosome-like nanovesicles and/or exosomes for the preparation of a composition to promote or enhance hair growth. In some embodiments, the present disclosure provides for the use of a composition comprising Withania somnifera exosome-like nanovesicles and/or exosomes to prevent or slow hair loss. In some embodiments, the present disclosure provides for the use of Withania somnifera exosome-like nanovesicles and/or exosomes for the preparation of a composition to prevent or slow hair loss.

In some embodiments, the present disclosure is directed to a composition comprising exosome-like nanovesicles and/or exosomes derived from Withania somnifera (Ashwaganda) and a carrier. In some embodiments, the present disclosure is directed to a composition for treating a hair follicle in a mammal comprising exosome-like nanovesicles and/or exosomes derived from derived from Withania somnifera. In some embodiments, the present disclosure is directed to a composition comprising exosome-like nanovesicles and/or exosomes derived from Withania somnifera, wherein the exosome-like nanovesicles and/or exosomes originate from one or more of the Withania somnifera stem, the Withania somnifera root, the Withania somnifera leaf, the Withania somnifera fruit, and the Withania somnifera seed. In some embodiments, the present disclosure is directed to a composition comprising exosome-like nanovesicles and/or exosomes derived from Withania somnifera, wherein the exosomes originate from the Withania somnifera stem. In some embodiments, the present disclosure is directed to a composition comprising exosome-like nanovesicles and/or exosomes derived from Withania somnifera, wherein the exosome-like nanovesicles and/or exosomes originate from the Withania somnifera root. In some embodiments, the present disclosure is directed to a composition comprising exosome-like nanovesicles and/or exosomes derived from Withania somnifera, wherein the exosomes originate from the Withania somnifera leaf. In some embodiments, the present disclosure is directed to a composition comprising exosome-like nanovesicles and/or exosomes derived from Withania somnifera, wherein the exosome-like nanovesicles and/or exosomes originate from the Withania somnifera seeds. In some embodiments, the present disclosure is directed to a composition comprising exosomes derived from Withania somnifera, wherein the exosome-like nanovesicles and/or exosomes originate from the Withania somnifera fruit.

In some embodiments, the present disclosure is directed to a composition comprising exosome-like nanovesicles and/or exosomes derived from Withania somnifera, wherein the exosome-like nanovesicles and/or exosomes originate from dried Withania somnifera. In some embodiments, the present disclosure is directed to a composition comprising exosome-like nanovesicles and/or exosomes derived from Withania somnifera, wherein the exosome-like nanovesicles and/or exosomes originate from dried Withania somnifera stem. In some embodiments, the present disclosure is directed to a composition comprising exosome-like nanovesicles and/or exosomes derived from Withania somnifera, wherein the exosome-like nanovesicles and/or exosomes originate from dried Withania somnifera root. In some embodiments, the present disclosure is directed to a composition comprising exosome-like nanovesicles and/or exosomes derived from Withania somnifera, wherein the exosome-like nanovesicles and/or exosomes originate from dried Withania somnifera leaf. In some embodiments, the present disclosure is directed to a composition comprising exosome-like nanovesicles and/or exosomes derived from Withania somnifera, wherein the exosome-like nanovesicles and/or exosomes originate from dried Withania somnifera fruit. In some embodiments, the present disclosure is directed to a composition comprising exosome-like nanovesicles and/or exosomes derived from Withania somnifera, wherein the exosome-like nanovesicles and/or exosomes originate from dried Withania somnifera seeds.

In some embodiments, the present disclosure is directed to a composition comprising exosome-like nanovesicles and/or exosomes derived from Withania somnifera for the treatment or prevention of sparse hair growth, short hair growth, thin hair growth, partial or complete hair loss on the scalp, alopecia, androgenic alopecia, alopecia androgenetica, male pattern baldness, female pattern baldness, non-androgenic alopecia, alopecia areata, alopecia totalis, alopecia universalis, radiation induced alopecia, alopecia due to radiotherapy, drug induced alopecia, alopecia due to chemotherapy, traumatic alopecia, scarring alopecia, psychogenic alopecia, stress related alopecia, cortisol related alopecia or anagen effluvium.

In some embodiments, the present disclosure is directed to a composition comprising exosome-like nanovesicles and/or exosomes derived from Withania somnifera, wherein the Withania somnifera is heat shocked before obtaining the exosome-like nanovesicles and/or exosomes. In some embodiments, the present disclosure is directed to a composition comprising exosome-like nanovesicles and/or exosomes derived from Withania somnifera, wherein the Withania somnifera is not heat shocked before obtaining the exosome-like nanovesicles and/or exosomes.

In some embodiments, the present disclosure is directed to a composition comprising exosome-like nanovesicles and/or exosomes derived from Withania somnifera, wherein the composition is applied topically. In some embodiments, the present disclosure is directed to a composition comprising exosome-like nanovesicles and/or exosomes derived from Withania somnifera, wherein the composition is an ointment or a cream.

In some embodiments, the present disclosure is directed to a composition comprising exosome-like nanovesicles and/or exosomes derived from Withania somnifera for use in a method of promoting hair growth or reducing hair loss comprising administering to a subject an effective amount of the composition of any one of the preceding claims, in a suitable vehicle, for a time sufficient to promote hair regrowth and reduce hair loss in subjects in need thereof. In some embodiments, the present disclosure is directed to a composition comprising exosome-like nanovesicles and/or exosomes derived from Withania somnifera for use in a method of preventing, reducing or reversing hair loss comprising administering to a subject in need thereof an effective amount of a Withania somnifera-derived exosome-like nanovesicles and/or exosomes or composition, wherein the hair loss is caused or mediated by cortisol or stress.

In some embodiments, the present disclosure is directed to a composition comprising exosome-like nanovesicles and/or exosomes derived from Withania somnifera for use in a method of promoting hair growth or reducing hair loss in a subject in need thereof comprising treating human dermal papilla with Withania somnifera-derived exosome-like nanovesicles and/or exosomes, or a composition thereof, having increased levels of heat shock stress-response molecules.

In some embodiments, the present disclosure is directed to a composition comprising exosome-like nanovesicles and/or exosomes derived from Withania somnifera for use in a method of promoting hair growth or reducing hair loss in a subject in need thereof comprising treating human dermal papilla with Withania somnifera-derived exosome-like nanovesicles and/or exosomes, or a composition thereof, having increased levels of heat shock stress-response molecules wherein the exosome-like nanovesicles and/or exosomes are obtained from one or more of the Withania somnifera stem, the Withania somnifera root, the Withania somnifera leaf, the Withania somnifera fruit, and the Withania somnifera seed conditioned by growing the Withania somnifera plant under heat shock conditions, and wherein the heat shock conditions comprise heating the Withania somnifera plant.

In some embodiments, the present disclosure is directed to a composition comprising exosome-like nanovesicles and/or exosomes derived from Withania somnifera, wherein the heat shock conditions comprise heating the Withania somnifera plant to a temperature of about 33° C. to about 45° C.

In some embodiments, the present disclosure is directed to a composition comprising exosome-like nanovesicles and/or exosomes derived from Withania somnifera, wherein the heat shock conditions comprise heating the Withania somnifera plant for about 1 hour to about 5 hours.

In some embodiments, the present disclosure is directed to a composition comprising exosome-like nanovesicles and/or exosomes derived from Withania somnifera, wherein the heat shock conditions comprise heating the Withania somnifera plant to a temperature of about 33° C. to about 45° C. for about 1 hour to about 5 hours.

In some embodiments, the present disclosure is directed to a composition comprising exosome-like nanovesicles and/or exosomes derived from Withania somnifera, wherein the heat shock conditions comprise heating the Withania somnifera nifera plant to a temperature of about 45° C.

In some embodiments, the present disclosure is directed to a composition comprising exosome-like nanovesicles and/or exosomes derived from Withania somnifera, wherein the Withania somnifera plant is primed by warming the Withania somnifera plant prior to the by heat shock conditions.

In some embodiments, the present disclosure is directed to a composition comprising exosome-like nanovesicles and/or exosomes derived from Withania somnifera, wherein the Withania somnifera-derived exosomes are within a cosmetic composition.

In some embodiments, the present disclosure is directed to a composition comprising exosome-like nanovesicles and/or exosomes derived from Withania somnifera, wherein the Withania somnifera-derived exosomes are comprised within a cosmetic composition and are applied topically to the scalp.

In some embodiments, the present disclosure is directed to a use of Withania somnifera-derived exosome-like nanovesicles and/or exosomes in the preparation of a cosmetic composition for promoting hair growth or reducing hair loss in a subject in need thereof.

In some embodiments, the present disclosure is directed to a composition comprising exosome-like nanovesicles and/or exosomes derived from Withania somnifera, wherein the Withania somnifera-derived exosomes, or composition thereof, is effective at preventing or reversing cortisol induced growth arrest in human follicle dermal papilla cells.

In some embodiments, the present disclosure is directed to a composition comprising exosome-like nanovesicles and/or exosomes derived from Withania somnifera, wherein the Withania somnifera-derived exosomes, or composition thereof, is effective at promoting growth factor secretion in wounded human follicle dermal papilla cells.

In some embodiments, the present disclosure is directed to a composition comprising exosome-like nanovesicles and/or exosomes derived from Withania somnifera, wherein the Withania somnifera-derived exosomes, or composition thereof, is effective at inducing melanogenesis in human primary melanocytes.

In some embodiments, the present disclosure is directed to a composition comprising exosome-like nanovesicles and/or exosomes derived from Withania somnifera, wherein the Withania somnifera-derived exosomes, or composition thereof, is effective at prolonging anagen phase in hair follicles.

In some embodiments, the present disclosure is directed to a composition comprising exosome-like nanovesicles and/or exosomes derived from Withania somnifera, wherein the Withania somnifera is dried before the exosomes are extracted.

In some embodiments, the present disclosure is directed to a composition comprising exosomes derived from Withania somnifera, wherein the Withania somnifera seeds are dried before the exosomes are extracted from the seeds.

In some embodiments, the present disclosure is directed to a composition comprising exosome-like nanovesicles and/or exosomes derived from Withania somnifera, wherein the Withania somnifera is freeze dried before the exosomes are extracted.

In some embodiments, the present disclosure is directed to a composition comprising exosome-like nanovesicles and/or exosomes derived from Withania somnifera, wherein the Withania somnifera seeds are freeze dried before the exosomes are extracted from the seeds.

In some embodiments, the composition further comprises an additional exosome-like nanovesicle or exosome. In some embodiments, the composition further comprises an additional exosome-like nanovesicle or exosome derived from aloe. In some embodiments, the composition further comprises a human exosome.

In some embodiments, any or all of the embodiments as discussed herein can be used with each other separately and in combination.

In one aspect, the present disclosure is directed to a composition comprising exosome-like nanovesicles and/or exosomes derived or extracted from Withania somnifera (Ashwaganda) and a carrier.

In another aspect, the present disclosure is directed to a composition for treating a hair follicle in a mammal comprising exosome-like nanovesicles and/or exosomes derived or extracted from Withania somnifera and a carrier.

In some embodiments, the composition of the present disclosure comprises exosome-like nanovesicles and/or exosomes that are extracted from one or more of the Withania somnifera stem, the Withania somnifera root, the Withania somnifera leaf, the Withania somnifera fruit, and the Withania somnifera seed.

In some embodiments, the composition of the present disclosure comprises exosome-like nanovesicles and/or exosomes extracted from the Withania somnifera seed.

In some embodiments, the Withania somnifera is heat shocked before extracting the exosome-like nanovesicles and/or exosomes.

In some embodiments, the Withania somnifera is not heat shocked before extracting the exosome-like nanovesicles and/or exosomes.

In some embodiments, the composition of the present disclosure treats, prevents or reverses sparse hair growth, short hair growth, thin hair growth, partial or complete hair loss on the scalp, alopecia, androgenic alopecia, alopecia androgenetica, male pattern baldness, female pattern baldness, non-androgenic alopecia, alopecia areata, alopecia totalis, alopecia universalis, radiation induced alopecia, alopecia due to radiotherapy, drug induced alopecia, alopecia due to chemotherapy, traumatic alopecia, scarring alopecia, psychogenic alopecia, stress related alopecia, cortisol related alopecia or anagen effluvium.

In some embodiments, the composition of the present disclosure is applied topically.

In some embodiments, the composition of the present disclosure is a liquid, and ointment or a cream. In some embodiments, the composition of the present disclosure is a liquid.

In some embodiments, the composition of the present disclosure is a cosmetic composition. In some embodiments, the composition of the present disclosure is a pharmaceutical composition.

In some embodiments, the composition of the present disclosure is a cosmetic composition and is applied topically to the scalp.

In some embodiments, the composition of the present disclosure is effective at preventing or reversing cortisol induced growth arrest in human follicle dermal papilla cells.

In some embodiments, the composition of the present disclosure comprises exosomes extracted from Withania somnifera which are dried before the exosome-like nanovesicles and/or exosomes are extracted.

In some embodiments, the composition of the present disclosure comprises exosomes extracted from Withania somnifera which are dried before the exosome-like nanovesicles and/or exosomes are extracted from the seeds.

In some embodiments, the composition of the present disclosure comprises exosomes extracted from Withania somnifera which are freeze dried before the exosome-like nanovesicles and/or exosomes are extracted.

In some embodiments, the composition of the present disclosure comprises exosomes extracted from Withania somnifera which are freeze dried before the exosome-like nanovesicles and/or exosomes are extracted from the seeds.

In some embodiments, the composition of the present disclosure further comprises an additional exosome-like nanovesicle or exosome.

In some embodiments, the composition of the present disclosure further comprises an additional exosome-like nanovesicle or exosome derived from aloe.

In some embodiments, the composition of the present disclosure further comprises a human exosome.

In some embodiments, the number of Withania somnifera exosome-like nanovesicles and/or exosomes within the composition is between about 1×10⁷ per mL to about 1×10¹² per mL.

In some embodiments, the number of extracted Withania somnifera exosome-like nanovesicles and/or exosomes within the composition is about 1×10⁷ per mL, about 1×10⁸ per mL, about 1×10⁹ per mL, about 1×10¹⁰ per mL, about 1×10¹¹ per mL, or about 1×10¹² per mL. In some embodiments, the number of Withania somnifera exosome-like nanovesicles and/or exosomes within the composition is from about 1×10⁹ per mL to about 1×10¹⁰ per mL.

In some embodiments, the number of Withania somnifera exosome-like nanovesicles and/or exosomes within the composition is about 1×10⁹ per mL.

In some embodiments, the number of Withania somnifera exosome-like nanovesicles and/or exosomes within the composition is about 1×10¹⁰ per mL.

In some embodiments, the composition further comprises aloe derived exosome-like nanovesicles and/or exosomes, and the number of aloe derived exosome-like nanovesicles and/or exosomes within the composition is from about 1×10⁷ per mL to about 1×10¹² per mL.

In some embodiments, the composition further comprises aloe derived exosome-like nanovesicles and/or exosomes, and the number of aloe derived exosome-like nanovesicles and/or exosomes within the composition is about 1×10⁷ per mL, about 1×10⁸ per mL, about 1×10⁹ per mL, about 1×10¹⁰ per mL, about 1×10¹¹ per mL, or about 1×10¹² per mL.

In some embodiments, the composition further comprises aloe derived exosome-like nanovesicles and/or exosomes, and the number of aloe derived exosome-like nanovesicles and/or exosomes within the composition is from about 1×10⁹ per mL to about 1×10¹⁰ per mL.

In some embodiments, the composition further comprises aloe derived exosome-like nanovesicles and/or exosomes, and the number of aloe derived exosome-like nanovesicles and/or exosomes within the composition is about 1×10⁹ per mL.

In some embodiments, the composition further comprises aloe-derived/extracted exosome-like nanovesicles and/or exosomes, and the number of aloe derived exosome-like nanovesicles and/or exosomes within the composition is about 1×10¹⁰ per mL.

In some embodiments, the exosome-like nanovesicles and/or exosomes are extracted and isolated from Withania somnifera.

In some embodiments, the exosome-like nanovesicles and/or exosomes are extracted or isolated from Withania somnifera, wherein the exosome-like nanovesicles and/or exosomes are purified.

In some embodiments, the composition of the present disclosure comprises a carrier.

In some embodiments, the carrier comprises water.

In some embodiments, the carrier comprises an aqueous solution.

In some embodiments, the composition of the present disclosure further comprises one or more of glycerin, Melaleuca alternifolia leaf water propanediol, 1,2-hexanediol, panthenol, niacinamide, hydroxyethylcellulose, Lepidium meyenii, root extract, maltodextrin, caprylhydroxamic acid, Hippophae rhamnoides fruit extract, Equisetum arvense extract, Laminaria saccharina extract, Chondrus crispus extract, sodium metabisulfite, alcohol, phospholipids, arginine, lactic acid, melatonin, potassium sorbate, lactobacillus ferment, Pisum sativum extract, and/or phosphate buffered saline.

In some embodiments, the composition of the present disclosure further comprises one or more of glycerin, Camellia sinensis (green tea) leaf extract, glycine, Larix europaea wood extract, sodium metabisulfite, zinc chloride, Pisum sativum (pea) sprout extract, alcohol, Olea europaea (olive) leaf extract, Curcuma longa (turmeric) root extract, Equisetum arvense (horsetail) extract, Hippophae rhamnoides (sea buckthorn) fruit oil, Laminaria saccharina (neptune kelp) extract, Lepidium meyenii (maca) root extract, Melaleuca alternifolia (tea tree) leaf oil, Moringa oleifera (moringa) leaf extract, Panax ginseng (ginseng) root extract, DL-panthenol, L-theanine, Melatonin, Niacinamide, sodium dehydroacetate, sodium hyaluronate, and/or phytic acid.

In some embodiments, the composition of the present disclosure further comprises one or more of water, glycerin, Melaleuca alternifolia leaf water, propanediol, butylene glycol, caffeine, 1,2-hexanediol, niacinamide, hydroxyethylcellulose, panthenol, Lepidium meyenii root extract, maltodextrin, caprylhydroxamic acid, Chondrus crispus extract, Hippophae rhamnoides fruit extract, Laminaria saccharina (neptune kelp) extract, an alcohol, phospholipids, sodium metabisulfite, arginine, lactic acid, melatonin, potassium sorbate, lactobacillus ferment, Pisum sativum extract, phosphate buffered saline, and/or Panax ginseng root extract.

In some embodiments, the composition of the present disclosure comprises a carrier comprising water, and the composition further comprises one or more of glycerin, an aqueous buffer, and a naturally occurring preservative.

In some embodiments, the composition of the present disclosure further comprises a naturally occurring preservative.

In some embodiments, the composition of the present disclosure further comprises a naturally occurring preservative, wherein the naturally occurring preservative comprises lactobacillus ferment.

In some embodiments, the composition of the present disclosure further comprises melatonin.

In some embodiments, the composition of the present disclosure further comprises niacinamide.

In some embodiments, the composition of the present disclosure further comprises an alcohol.

In some embodiments, the composition of the present disclosure further comprises ethyl alcohol.

In some embodiments, the composition of the present disclosure comprises about 0.01% to 10% by weight Withania somnifera exosome-like nanovesicles and/or exosomes.

In some embodiments, the composition of the present disclosure comprises about 0.1% to 5% by weight Withania somnifera exosome-like nanovesicles and/or exosomes.

In some embodiments, the composition of the present disclosure comprises about 0.1% to 4% by weight Withania somnifera exosome-like nanovesicles and/or exosomes.

In some embodiments, the composition of the present disclosure comprises about 0.1% to 3% by weight Withania somnifera exosome-like nanovesicles and/or exosomes.

In some embodiments, the composition of the present disclosure comprises about 0.1% to 2% by weight Withania somnifera exosome-like nanovesicles and/or exosomes.

In some embodiments, the composition of the present disclosure comprises about 0.1% to 1% by weight Withania somnifera exosome-like nanovesicles and/or exosomes.

In some embodiments, the composition of the present disclosure comprises about 0.3% to about 1% by weight Withania somnifera exosome-like nanovesicles and/or exosomes.

In another aspect, the present disclosure is directed to a method of promoting hair growth or reducing hair loss comprising administering to a subject an effective amount of the composition comprising extracted Withania somnifera exosome-like nanovesicles and/or exosomes in a suitable carrier, for a time sufficient to promote hair regrowth and reduce hair loss and/or promote hair growth in subjects in need thereof.

In another aspect, the present disclosure is directed to a method of preventing, reducing or reversing hair loss comprising administering to a subject in need thereof an effective amount of a composition comprising extracted Withania somnifera exosome-like nanovesicles and/or exosomes, for a time sufficient to prevent, reduce or reverse hair loss, wherein the hair loss is caused or mediated by cortisol or stress.

In another aspect, the present disclosure is directed to a method for effecting changes in mammalian hair appearance, hair growth, hair pigmentation and hair follicle and hair shaft size, comprising administering to the skin of a mammal an effective amount of a topically active composition comprising extracted Withania somnifera derived exosome-like nanovesicles and/or exosomes.

In another aspect, the present disclosure is directed to a method for producing a melanogenetic action in the hair and to promote its pigmentation and pigmentation of the stem, comprising the step of administering to a subject in need thereof an effective amount of a composition comprising extracted Withania somnifera derived exosome-like nanovesicles and/or exosomes.

In another aspect, the present disclosure is directed to a method of stimulating hair growth or preventing hair loss in a subject in need thereof, comprising topically applying a composition comprising between about 0.1% to about 5% extracted Withania somnifera exosome-like nanovesicles and/or exosomes and a carrier,

wherein the number of Withania somnifera exosome-like nanovesicles and/or exosomes within the composition is between about 1×10⁸ per mL and about 1×10¹⁰ per mL, and wherein the Withania somnifera exosome-like nanovesicles and/or exosomes are extracted from dried Withania somnifera seeds.

In another aspect, the present disclosure is directed to a method of promoting hair growth or reducing hair loss in a subject in need thereof comprising,

treating human dermal papilla with Withania somnifera-derived exosome-like nanovesicles and/or exosomes, or a composition thereof, having increased levels of heat shock stress-response molecules,

wherein the exosomes are extracted from one or more of the Withania somnifera stem, the Withania somnifera root, the Withania somnifera leaf, the Withania somnifera fruit, and the Withania somnifera seed conditioned by growing the Withania somnifera plant under heat shock conditions, and

wherein the heat shock conditions comprise heating the Withania somnifera plant.

In some embodiments, the heat shock conditions comprise heating the Withania somnifera plant to a temperature of about 33° C. to about 45° C.

In some embodiments, the heat shock conditions comprise heating the Withania somnifera plant for about 1 hour to about 5 hours.

In some embodiments, the heat shock conditions comprise heating the Withania somnifera plant to a temperature of about 33° C. to about 45° C. for about 1 hour to about 5 hours.

In some embodiments, the heat shock conditions comprise heating the Withania somnifera plant to a temperature of about 45° C.

In some embodiments, the Withania somnifera plant is primed by warming the Withania somnifera plant prior to the by heat shock conditions.

In another aspect, the present disclosure is directed to a kit for promoting hair growth or preventing, reducing, or reversing hair loss comprising (i) a composition comprising Withania somnifera exosome-like nanovesicles and/or exosomes and (ii) instructions for topically applying the composition to the scalp of a subject in need thereof.

In another aspect, the present disclosure is directed to the use of extracted Withania somnifera-derived exosome-like nanovesicles and/or exosomes in the preparation of a cosmetic composition for promoting hair growth or preventing, reducing, or reversing hair loss in a subject in need thereof.

In some embodiments, the present disclosure is directed to plant-derived exosome-like nanovesicles and/or exosomes, formulations/compositions, and methods of use thereof. In some embodiments, the present disclosure is directed to method of treating or a method of preventing hair loss using a plant-derived exosome-like nanovesicles and/or exosomes or a formulation/composition thereof.

In some embodiments, the exosome-like nanovesicles and/or exosomes of the present disclosure are derived from Withania somnifera (Ashwagandha). In some embodiments, the plant source for the exosome-like nanovesicles and/or exosomes of the present disclosure is Withania somnifera. In some embodiments, the exosome-like nanovesicles and/or exosomes are derived from one or more of the Withania somnifera root, Withania somnifera stem, Withania somnifera leaf, Withania somnifera fruit, and/or Withania somnifera seed. In some embodiments, the exosome-like nanovesicles and/or exosomes are derived from one or more of the heat shocked Withania somnifera root, heat shocked Withania somnifera stem, heat shocked Withania somnifera leaf, heat shocked Withania somnifera fruit, and heat shocked Withania somnifera seed.

In some embodiments, the Withania somnifera-derived exosome-like nanovesicles and/or exosomes are within a composition. In some embodiments, the Withania somnifera exosome-like nanovesicles and/or exosomes are within a cosmetic composition. In some embodiments, the Withania somnifera exosome-like nanovesicles and/or exosomes are within a cosmetic composition and applied topically. In some embodiments, the Withania somnifera-derived exosome-like nanovesicles and/or exosomes are used for the treatment of various types of hair loss.

In some embodiments, the present disclosure provides for the use of a composition comprising Withania somnifera exosome-like nanovesicles and/or exosomes to promote or enhance hair growth. In some embodiments, the present disclosure provides for the use of Withania somnifera exosome-like nanovesicles and/or exosomes for the preparation of a composition to promote or enhance hair growth. In some embodiments, the present disclosure provides for the use of a composition comprising Withania somnifera exosome-like nanovesicles and/or exosomes to prevent or slow hair loss. In some embodiments, the present disclosure provides for the use of Withania somnifera exosome-like nanovesicles and/or exosomes for the preparation of a composition to prevent or slow hair loss.

In some embodiments, the present disclosure is directed to a method for effecting changes in mammalian hair appearance, hair growth, hair pigmentation and hair follicle and hair shaft size, comprising topical application to the skin of a mammal an effective amount of a topically active composition comprising a Withania somnifera derived exosome-like nanovesicles and/or exosomes or compositions thereof. In some embodiments all the methods and compositions herein are useful for the reduction of grey and white hair.

In some embodiments, the present disclosure is directed to a method for producing a melanogenetic action in the hair and to promote its pigmentation and pigmentation of the skin, comprising the step of administering to a subject in need thereof an effective amount of a Withania somnifera derived exosome-like nanovesicles and/or exosomes or compositions thereof.

In some embodiments, the Withania somnifera derived exosome-like nanovesicles and/or exosomes are given in combination with one or more additional agents. In some embodiments, the one or more additional agents are selected from, an additional plant-derived exosome, a human exosome, a human exosome-like nanovesicle, and/or an agent that prevents or reverses hair loss. In some embodiments, the Withania somnifera derived exosome-like nanovesicles and/or exosomes are given in combination with a human exosome. In some embodiments, the Withania somnifera derived exosome-like nanovesicles and/or exosomes are given in combination with a plant derived exosome-like nanovesicle. In some embodiments, the Withania somnifera derived exosome-like nanovesicles and/or exosomes are given in combination with an aloe derived exosome-like nanovesicle and/or exosomes.

### Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, illustrative materials and methods are described herein. Accordingly, the terminology used herein is for the purpose of describing particular embodiments only, and is not intended to be limiting.

In the detailed description herein, references to “some embodiments,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While the following terms are believed to be well understood in the context used herein, the following definitions are set forth to facilitate explanation of the presently disclosed subject matter.

As used herein, “a”, “an”, and “the” refer to “one or more” when used in this application, including the claims. Thus, for example, reference to “a carrier” includes mixtures of one or more carriers, two or more carriers, and the like.

As used herein, “about” or “approximately” shall generally mean within 20 percent, within 10 percent, or within 5 percent of a given value or range.

As used herein, “comprising” means that other steps and other ingredients which do not affect the end result can be added. This term encompasses the terms “consisting of” and “consisting essentially of”. As used herein, the verb “comprise” as is used in this description and in the claims and its conjugations are used in its non-limiting sense to mean that items following the word are included, but items not specifically mentioned are not excluded.

As used herein, “mixtures” is meant to include a simple combination of materials and any compounds that may result from their combination.

As used herein, “molecular weight” or “M. Wt.” refers to the weight average molecular weight unless otherwise stated.

As used herein, the terms “include,” “includes,” and “including,” are meant to be non-limiting and are understood to mean “comprise,” “comprises,” and “comprising,” respectively.

As used herein, “percentage” or “%” refer to concentrations by weight or by mass, unless defined otherwise.

As used herein, “mass,” “%”, “wt/wt,” or alternatively “weight,” mean the calculations of the mass of one or more components in a formulation divided by the total mass of the formulation. In some embodiments, the mass of each component and the total mass of the formulation can be determined by using analytical balances as is well known by those skilled in the art. In some embodiments, the mass or weight is determined on an as-is basis. In some embodiments, the calculations of the mass can include the mass of liquids present in the component and/or the formulation.

As used herein, the terms “subject,” “individual,” and “patient” are used interchangeably herein to refer to a vertebrate, in some embodiments a mammal, and in some embodiments a human. Mammals include, but are not limited to, murines, simians, humans, farm animals, sport animals, and pets. Tissues, cells and their progeny of a biological entity obtained in vivo or cultured in vitro are also encompassed.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, “alopecia” refers to partial or complete hair loss on the scalp, including, but not limited to sparse hair growth, short hair growth, thin hair growth, etc. Hair loss also occurs in a variety of in other conditions.

As used herein, the term “effective amount” or “therapeutically effective amount” refers to the amount of an agent that is sufficient to effect beneficial or desired results. The therapeutically effective amount may vary depending upon one or more of: the subject and condition being treated, the weight and age of the subject, the severity of the condition, the manner of administration and the like, which can readily be determined by one of ordinary skill in the art. The term also applies to a dose that will provide a detectable change by any one of the methods described herein. The specific dose may vary depending on one or more of: the particular agent chosen, the dosing regimen to be followed, whether it is administered in combination with other compounds, timing of administration, the desired effect, and the physical delivery system in which it is carried. In some embodiments, the composition is a certain concentration, and the concentration is in exosome-like nanovesicles and/or exosomes per mL. In some embodiments, the exosome-like nanovesicles and/or exosomes per mL are between 1×10⁷ and 1×10¹².

As used herein, “plant-derived exosomes”, “exosome-like nanovesicles (ELNVs or ELNs)”, “plant derived exosome-like nanovesicles”, “plant derived exosome-like nanovesicles” refer to are biological nanostructures which are secreted by most types of cells and relay information between cells and organisms to regulate physiological functions of multicellular organisms in an intercellular transmission manner. The terms “plant-derived exosomes”, “exosome-like nanovesicles (ELNVs)”, “plant derived exosome-like nanovesicles”, “plant derived exosome-like nanovesicles”, “exosomes”, “exosome-like vesicles”, “microvesicles”, “secreted microvesicles”, “extracellular vesicles”, and “secreted vesicles” are used interchangeably herein for the purposes of the specification and claims.

As used herein, “Withania somnifera exosomes”, “Withania somnifera nanovesicles”, “Withania somnifera exosome-like nanovesicles”, and “ASH-NV” refer to the species that are extracted from Withania somnifera according to the Examples herein.

As used herein, the term “heat shock” and “stress-response molecules” are used interchangeably herein for the purposes of the specification and claims. These terms are meant to include molecules present in exosomes that are secreted by plant cells subjected to high temperature (otherwise known as “heat shock”).

As used herein, the term “extract” or “isolated” are used interchangeably herein and includes separating one or more substances (e.g. Withania somnifera exosome-like nanovesicles) from a mixture (e.g. a Withania somnifera plant). This includes those substances “derived from” (e.g. Withania somnifera exosome-like nanovesicles) a particular source. The term “derived from” assumes that the component is extracted from the source (e.g. a Withania somnifera plant). In some circumstances, extraction can be accomplished via chemical methods, physical methods (e.g. centripetal force, size exclusion, etc.), or other means of removing or taking a substance out of a mixture of two or more components or substances.

“Eyebrow” as used in this document refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris such as dandruff and other small objects from falling into the eyes, as well as providing a more sensitive sense for detecting objects being near the eye, like small insects. Eyebrows also have an important facilitative function in communication, strengthening facial expressions such as surprise, confusion, or anger.

The terms “eyelash” and “lash” are used interchangeably to refer to one of the hairs that grow at the edge of the eyelid. Eyelashes protect the eye from debris and provide a warning that an object (such as an insect or dust mite) is near the eye (which then is closed reflexively).

In some embodiments, isolated exosomes can be prepared from Withania somnifera in a controlled environment, wherein the plant is exposed to various stimuli to manipulate the exosomal cargo. In an example of providing exosomes engineered for promoting hair growth or preventing hair loss, Withania somnifera can be subjected to relatively high temperature (otherwise known as “heat shock”) to produce exosomes having increased levels of heat shock stress-response molecules, including stress-response proteins. In some embodiments, the stress-response proteins are in the HSP70 protein family. HSP70 proteins are a family of proteins expressed in response to heat stress or heat shock. HSP70 proteins have three major functional domains: N-terminal ATPase domain, substrate binding domain, and C-terminal domain.

As used herein, the term “increased levels” of heat shock stress-response molecules means that the amount of stress-response molecules present in exosomes of a plant that has been subjected to a relatively high temperature (or heat shock) is higher than the amount of stress-response molecules present in exosomes of a plant subjected to conventional plant exposure temperatures (for example, room temperature, which is generally around 25° C.). For example, increased levels may include increases of 5% to 200% relative to plants having no heat shock treatment. For example, the level of heat shock stress-response molecules in exosomes of a heat shocked plant may be 5%, 10%, 20%, 30%, 40%, 50%, 60%, 70%, 80%, 90%, 100%, 125%, 150%, 175% or 200% greater than the level of heat shock stress-response molecules in exosomes of non-heat shocked plants. Additionally, the level of heat shock stress-response molecules in exosomes of a heat shocked plant may be 2, 3, 4, 5, 10, 15, 20, 25, or 30 times greater than the level of heat shock stress-response molecules in exosomes of non-heat shocked plants.

As used herein, “exosome” or “exosomes” may refer to the Withania somnifera derived exosomes of the present disclosure.

In some embodiments, the present disclosure is directed to a Withania somnifera-derived exosome-containing composition comprising isolated Withania somnifera-based exosomes containing heat shock stress-response molecules and a carrier. The heat shock stress-response molecules can be any molecules present in the plant exosomes that are secreted by Withania somnifera cells in response to being subjected to a growing temperature that is relatively higher than the growing temperature to which the plant was exposed previously. Heat shock stress-response molecules are typically proteins produced by cells in response to exposure to stressful conditions, such as heat shock. Heat-shock proteins are named according to their molecular weight. For example, Hsp60, Hsp70 and Hsp90 refer to families of heat shock proteins on the order of 60, 70, and 90 kilodaltons in size, respectively.

In some embodiments, a cell population can comprise one or more cell types, notably 2 or more cell types, 3 or more cell types, 4 or more cell types, or 5 or more cell types. In some embodiments, a cell population comprises at least 1 to 40 cell types, notably at least 1 to 30, at least 5 to 20, at least 5 to 10, at least 2 to 8 or at least 2 to 5 cell types. Therefore, cell type or cell subtype exosomes can be purified from a mixed exosome population obtained from a cell population.

A plant may be accustomed to being grown at a temperature of about room temperature, which is about 25° C. Thus, any growing temperature higher than 25° C. could be a relatively higher temperature. A plant may also be accustomed to a growing temperature that is higher or lower than room temperature.

A relatively higher growing temperature may include a temperature at least 10° C. greater than the growing temperature to which the plant was previously exposed. Using room temperature as an example, a relatively higher growing temperature may include 35, 36, 37, 38, 39, 40, 41, 42, 43, 44, 45, 46, 47, 48, 49, or 50° C. Increasing the growing temperature of the plant may include increasing the temperature to a range of about 30° C. to about 45° C., about 30° C. to about 40° C., about 32° C. to about 38° C., about 33° C. to about 37° C., and/or about 40° C. to about 45° C.

A plant can be subjected to a relatively higher growing temperature for various periods of time. For example, a plant can be subjected to a relatively higher temperature for a period of time of about 30 minutes to about 6 hours, including, for example, 30 minutes, 60 minutes, 90 minutes, 2 hours, 2.5 hours, 3 hours, 3.5 hours, 4 hours, 4.5 hours, 5 hours 5.5 hours or 6 hours. After the plant is subjected to a relatively higher growing temperature for a period of time (i.e., heat shocked), the plant may then be exposed to a relatively lower growing temperature for a period of time. For example, the plant may be exposed to a temperature of about 25° C. to about 27° C. for about 24 hours to about 72 hours subsequent to heat shocking.

### Exosomes

Exosomes are small membrane vesicles formed in late endocytic compartments (multivesicular bodies) first described to be secreted by reticulocytes in 1983 and subsequently found to be secreted by many cell types including various haematopoietic cells, tumors of haematopoietic or non-haematopoietic origin and epithelial cells. They are distinct entities from the more recently described ‘ribonuclease complex’ also named exosome.

Exosomes may be defined by a number of morphological and biochemical parameters. Accordingly, the exosome described here may comprise one or more of these morphological or biochemical parameters.

Exosomes are classically defined as “saucer-like” vesicles or a flattened sphere sometimes limited by a lipid bilayer. The molecular composition of exosomes from different cell types and of different species has been examined. In general, exosomes contain ubiquitous proteins that appear to be common to all exosomes and proteins that are cell-type specific. Also, proteins in exosomes from the same cell-type but of different species are highly conserved. The ubiquitous exosome-associated proteins include cytosolic proteins found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. The tetraspannins are highly enriched in exosomes and are known to be involved in the organization of large molecular complexes and membrane subdomains.

Exosomes are also known to contain mRNA and microRNA, which can be delivered to another cell, and can be functional in this new location. The physiological functions of exosome remain poorly defined. It is thought to help eradicate obsolete proteins, recycle proteins, mediate transmission of infectious particles such as prions and viruses, induce complement resistance, facilitate immune cell-cell communication and transmit cell signaling. Exosomes have been used in immunotherapy for treatment of cancer.

The exosome-like nanovesicles and/or exosomes may have a molecular weight of greater than 100 kDa. It may have a molecular weight of greater than 500 kDa. For example, it may have a molecular weight of greater than 1000 kDa.

The molecular weight may be determined by various means. In principle, the molecular weight may be determined by size fractionation and filtration through a membrane with the relevant molecular weight cut-off. The exosome size may then be determined by tracking segregation of component proteins with SDS-PAGE or by a biological assay.

### Assay of Molecular Weight by SDS-PAGE

The Withania somnifera derived exosome-like nanovesicles and/or exosomes may have a molecular weight of greater than 100 kDa. For example, the Withania somnifera derived exosome-like nanovesicles and/or exosomes may be such that most proteins of the exosome with less than 100 kDa molecular weight segregate into the greater than 100 kDa molecular weight retentate fraction, when subject to filtration. Similarly, when subjected to filtration with a membrane with a 500 kDa cut off, most proteins of the Withania somnifera derived exosome-like nanovesicles and/or exosomes with less than 500 kDa molecular weight may segregate into the greater than 500 kDa molecular weight retentate fraction. This indicates that the Withania somnifera derived exosome-like nanovesicles and/or exosomes may have a molecular weight of more than 500 kDa.

### Exosome Size

The Withania somnifera derived exosome-like nanovesicles and/or exosomes may have a size of greater than 1 nm. The Withania somnifera derived exosome-like nanovesicles and/or exosomes may have a size of greater than 5 nm, 10 nm, 20 nm, 30 nm, 40 nm, 50 nm, 60 nm, 70 nm, 80 nm, 90 nm, 100 nm, 150 nm, 200 nm, 250 nm, 300 nm. The Withania somnifera derived exosome-like nanovesicles and/or exosomes may have a size of greater than 100 nm, such as greater than 150 nm. The Withania somnifera derived exosome-like nanovesicles and/or exosomes may have a size of substantially 200 nm or greater. The Withania somnifera derived exosome-like nanovesicles and/or exosomes may have a range of sizes, such as between 1 nm to 20 nm, 1 nm to 50 nm, 1 nm to 100 nm, 1 nm to 150 nm or 1 nm to 200 nm. The Withania somnifera derived exosome-like nanovesicles and/or exosomes may have a size between 10 nm to 50 nm, 10 nm to 100 nm, 10 nm to 150 nm, or 10 nm to 200 nm. The Withania somnifera derived exosome-like nanovesicles and/or exosomes may have a size between 50 nm to 100 nm, 50 nm to 150 nm or 50 nm to 200 nm. The Withania somnifera derived exosome-like nanovesicles and/or exosomes may have a size between 100 nm to 150 nm or 100 nm to 200 nm. The Withania somnifera derived exosome-like nanovesicles and/or exosomes may have a size between 150 nm to 200 nm. The Withania somnifera derived exosome-like nanovesicles and/or exosomes may have a size between 50 nm to 250 nm. The Withania somnifera derived exosome-like nanovesicles and/or exosomes may have a size between 100 nm to 250 nm. The Withania somnifera derived exosome-like nanovesicles and/or exosomes may have a size between 150 nm to 250 nm. The Withania somnifera derived exosome-like nanovesicles and/or exosomes may have a size between 200 nm to 250 nm. The Withania somnifera derived exosome-like nanovesicles and/or exosomes may have a size between 50 nm to 500 nm. The Withania somnifera derived exosome-like nanovesicles and/or exosomes may have a size between 100 nm to 500 nm. The Withania somnifera derived exosome-like nanovesicles and/or exosomes may have a size between 150 nm to 500 nm. The Withania somnifera derived exosome-like nanovesicles and/or exosomes may have a size between 200 nm to 500 nm. The Withania somnifera derived exosome-like nanovesicles and/or exosomes may have a size between 250 nm to 500 nm.

The size may be determined by various means. In principle, the size may be determined by size fractionation and filtration through a membrane with the relevant size cut-off. The exosome-like nanovesicles and/or exosomes size may then be determined by tracking segregation of component proteins with SDS-PAGE or by a biological assay.

The size may also be determined by electron microscopy.

In some embodiments, the size of the Withania somnifera derived exosome-like nanovesicles and/or exosomes may comprise a hydrodynamic radius. In some embodiments, hydrodynamic radius of the Withania somnifera derived exosome-like nanovesicles and/or exosomes may be below 100 nm, below 150 nm, below 200 nm, below 250 nm, below 300 nm, below 350 nm, below 400 nm, below 450 nm, or below 500 nm. The hydrodynamic radius of the exosome-like nanovesicles and/or exosomes may be below 150 nm. The hydrodynamic radius of the exosome-like nanovesicles and/or exosomes may be below 100 nm.

The hydrodynamic radius of the exosome-like nanovesicles and/or exosomes may be below 200 nm. The hydrodynamic radius of the exosome-like nanovesicles and/or exosomes may be below 150 nm. The hydrodynamic radius of the exosome-like nanovesicles and/or exosomes may be below 100 nm.

In some embodiments, the hydrodynamic radius of the Withania somnifera derived exosome-like nanovesicles and/or exosomes may be between 100 nm and 500 nm, between 150 nm and 500 nm, between 200 nm and 500 nm, between 250 nm and 500 nm, between 300 nm and 500 nm, between 350 nm and 500 nm, between 400 nm and 500 nm, between 450 nm and 500 nm.

In some embodiments, the hydrodynamic radius of the Withania somnifera derived exosome-like nanovesicles and/or exosomes may be between 100 nm and 400 nm, between 150 nm and 400 nm, between 200 nm and 400 nm, between 250 nm and 400 nm, between 300 nm and 400 nm, between 350 nm and 400 nm.

In some embodiments, the hydrodynamic radius of the Withania somnifera derived exosome-like nanovesicles and/or exosomes may be between 100 nm and 300 nm, between 150 nm and 300 nm, between 200 nm and 300 nm, or between 250 nm and 300 nm.

In some embodiments, the hydrodynamic radius of the Withania somnifera derived exosome-like nanovesicles and/or exosomes may be between 100 nm and 300 nm, between 150 nm and 300 nm, between 200 nm and 300 nm, or between 250 nm and 300 nm.

In some embodiments, the hydrodynamic radius of the Withania somnifera derived exosome-like nanovesicles and/or exosomes may be between 100 nm and 250 nm, between 150 nm and 250 nm, or between 200 nm and 250 nm.

In some embodiments, the hydrodynamic radius of the Withania somnifera derived exosome-like nanovesicles and/or exosomes may be between 100 nm and 200 nm, or between 150 nm and 200 nm.

In some embodiments, the hydrodynamic radius of the Withania somnifera derived exosome-like nanovesicles and/or exosomes may be between 10 nm and 200 nm, or between 50 nm and 200 nm.

In some embodiments, the hydrodynamic radius of the Withania somnifera derived exosome-like nanovesicles and/or exosomes may be between about 30 nm and about 70 nm. In some embodiments, the hydrodynamic radius of the Withania somnifera derived exosome-like nanovesicles and/or exosomes may be between about 40 nm and about 60 nm, such as between about 45 nm and about 55 nm. In some embodiments, the hydrodynamic radius of the Withania somnifera derived exosome-like nanovesicles and/or exosomes may be about 50 nm.

The hydrodynamic radius of the exosome-like nanovesicles and/or exosomes may be determined by any suitable means, for example, laser diffraction or dynamic light scattering. An example of a dynamic light scattering method to determine hydrodynamic radius is described in WO 2009/105044.

Ashwagandha (Withania somnifera)

Withania somnifera (Ashwaganda), also known as Indian ginseng, poison gooseberry, or winter cherry, is a plant in the Solanaceae or nightshade family and is a major herbal remedy in Ayurvedic medicine. Ashwagandha is known for the treatment and prevention of a range of diseases. The traditional use of this herb is as a tonic and activator. It is believed to prolong life, increase mental function and physical stamina, and improve sexual function. It also helps improve learning ability and memory capacity. Ashwagandha is considered to be an “adaptogen”: a substance that has the ability to help the body adjust to stressful situations. Adaptogens have effects on the human body that assist in maintaining equilibrium in response to physical, psychological, emotional or environmental stress. Accordingly, ashwagandha has been used for more than 2,500 years to address a range of medical issues including improving physical energy and endurance, improving immune function and providing resistance against ailments. Adaptogens such as ashwagandha can be utilized as supplements as part of a daily regimen to reduce psychological and physical stress in an individual. In the context of the present disclosure, the term “adaptogen” specifically refers an ingredient to combat stress in the body. Administration of an adaptogen such as ashwagandha is herein described as a method for reducing stress in the body in order to enhance the specific actions of a mixture of ingredients. The adaptogens are a unique class of herbal ingredients that result in the restoration of normal physiological function (homeostasis), and to increase the body's resistance to the effects of stress, such as by decreasing cellular sensitivity to stress. Ashwagandha is known to rebalance and lower the levels of the stress hormone cortisol, to improve thyroid function, and to elevate the body's endogenous antioxidant enzymes through its principal withanolides. Ashwagandha also exhibits inhibitory effects on pro-inflammatory cytokines such as IL-6 and TNF-α. The active compounds in Withania somnifera leaves and roots are C28 steroidal lactone molecules known as withanolides, such as Withaferin A, and are extracted from the plant using known methods, U.S. Pat. No. 7,108,870.

### Hair Growth

Methods of assessing promotion of hair growth are known in the art and are described below. A straightforward method for assessing improvement in hair growth is by taking a photograph of a test area of the skin before and after application of nano- or micro-emulsion composition. The skin may optionally be shaved for this purpose. A photograph is taken. The treatment is then applied. A second photograph is then taken. The increase in hair growth may be quantified by counting any combination of: (a) number of hairs appearing; (b) length of hair appearing; (c) thickness of hair appearing; (d) straightness of hair appearing; (e) area of hair growth. Where the skin is not shaved, the relevant measurements may be with regard to improvement in the measured parameters, i.e., number of new hairs, increase in length of hair, increase in thickness of hair, increase in straightness of hair and increase in area of hair growth.

For example, hair growth may be assessed in an individual. An individual to whom the composition is administered may display enhanced hair growth, as measured by any of the parameters described above, of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more. This may be compared to hair growth in an individual to which the composition is not administered. The enhanced hair growth may be assessed by the number of additional or the number of thick or the number of straight hairs. Otherwise, it may be assessed by the thickness of hair growth. It may be assessed by an increased area of hair growth.

The Withania somnifera derived exosome-like nanovesicles and/or exosomes or compositions thereof may be used to alleviate any type of alopecia. Examples of non-androgenic alopecia include alopecia areata, alopecia due to radiotherapy or chemotherapy, scarring alopecia, stress related alopecia, etc. As used in this application, “alopecia” refers to partial or complete hair loss on the scalp, including, but not limited to sparse hair growth, short hair growth, thin hair growth, etc. Hair loss also occurs in a variety of in other conditions.

Anagen effluvium, is hair loss due to chemicals or radiation, such as chemotherapy or radiation treatment for cancer. It is also commonly referred to as “drug induced” or “radiation induced” alopecia. The Withania somnifera derived exosome-like nanovesicles and/or exosomes or compositions thereof may be used to manufacture preparations to treat these types of alopecia.

Alopecia areata is an autoimmune disorder which initially presents with hair loss in a rounded patch on the scalp. It can progress to the loss of all scalp hair, which is known as alopecia totalis and to the loss of all scalp and body hair, which is known as alopecia universalis. The Withania somnifera derived exosome-like nanovesicles and/or exosomes or composition thereof may be used to manufacture preparations to treat these types of alopecia.

Traumatic alopecia is the result of injury to the hair follicle. It is also commonly referred to as “scarring alopecia”. Psychogenic alopecia occurs due to acute emotional stress. In some embodiments, and without being bound by any theory, by inducing anagen, the Withania somnifera derived exosome-like nanovesicles and/or exosomes or composition thereof can be beneficial in these types of alopecia as well. Thus, the uses of the Withania somnifera derived exosome-like nanovesicles and/or exosomes or composition thereof are not limited to treating androgenetic alopecia. The Withania somnifera derived exosome-like nanovesicles and/or exosomes or composition thereof can be used to manufacture preparations to alleviate any type of hair loss (by prolonging the anagen phase).

Thus, the Withania somnifera derived exosome-like nanovesicles and/or exosomes or composition thereof can be applied topically to the scalp and hair to prevent or alleviate balding. Further, the Withania somnifera derived exosome-like nanovesicles and/or exosomes or composition thereof can be applied topically in order to induce or promote the growth of hair on the scalp.

In some embodiments, the compositions described herein and methods described herein can be used to stimulate hair growth or prevent hair loss in any situation where additional hair growth is desired. In particular, the method of the present disclosure is useful when a subject has experienced hair loss associated with various conditions, including but not limited to: anagen effluvium, drug properties Alopecia, radiation therapy, poisoning, diffuse alopecia areata, alopecia areata, loose anagen syndrome, postoperative occipital alopecia, syphilis, traction alopecia (traction alopecia), tricholtillomania tinea capitis, resting hair loss, telogen gravidarum, chronic resting hair loss, early male onset alopecia, iron deficiency, malnutrition/dyspepsia, Hypothyroidism, hyperthyroidism, systemic lupus erythematosus, chronic renal failure, liver dysfunction, advanced malignancy, viral or bacterial infection, and male developmental alopecia. In particular, the methods of the present disclosure are useful for male developmental alopecia, alopecia areata, alopecia in drug-induced alopecia (e.g. following cancer chemotherapy), and recovery of alopecia resulting from radiation therapy.

In some embodiments, the Withania somnifera derived exosome-like nanovesicles and/or exosomes or composition thereof can be applied topically to the scalp and hair in order to prolong the anagen phase of the hair cycle. In some embodiments, the Withania somnifera derived exosome-like nanovesicles and/or exosomes or composition thereof can be applied topically to the scalp and hair in order to prevent or alleviate balding. In some embodiments, the Withania somnifera derived exosome-like nanovesicles and/or exosomes or composition thereof can be applied topically to the scalp and hair in order to induce or promote the growth of hair on the scalp. In some embodiments, the Withania somnifera derived exosome-like nanovesicles and/or exosomes or composition thereof can be used to manufacture preparations to simulate hair growth or prevent or alleviate any type of hair loss by prolonging the anagen phase.

### Hair Appearance and Pigmentation

Melanocytes present in the epidermis, in the bulb of the hair follicle and in the external root sheath of the hair follicle are mutually different. The major differences lie in the respective melanocyte-keratinocyte functional units. The melanin unit of the hair bulb is found in the bulb in the proximal anagen, which is an immunologically distinct region of the skin. Said unit comprises one melanocyte every 5 keratinocytes in the hair bulb, and one melanocyte every keratinocyte in the basal layer of the hair bulb matrix. Conversely, each epidermal melanocyte is associated with 36 vital keratinocytes in the immunocompetent epidermal melanin unit.

The most evident difference between these two melanocyte populations is that the activity of the melanocyte in the hair bulb is subjected to cycle control and, therefore, the corresponding melanogenesis is strictly associated with the growth cycle of hair and is, hence, discontinuous. Epidermal melanogenesis, instead, appears to be continuous.

In fact, the hair cycle includes periods of melanocyte proliferation (during the early anagen phase), maturation (from halfway through to the end of the anagen phase), and death of melanocytes by apoptosis (during the early catagen phase). Every hair cycle is associated with the reconstruction of a pigment unit that is intact at least for the first ten cycles (Tobin, Int. J. Cosmetic Science, 2008; Tobin and Paus, Exp. Gerontol., 2001). Biosynthesis of melanin and its subsequent transfer from melanocytes to keratinocytes in the hair bulb depend on the availability of melanin precursors and on complex signal transduction mechanisms.

Though follicular and epidermal melanocytes have common traits, follicular melanocytes seem to be more sensitive than epidermal ones to the aging process. The pigmentary unit of hair plays an important role as environmental sensor, and also an important physiological function. In practice, pigments contribute to the is rapid excretion of heavy metals and toxins from the body through their selective bond with melanin (Tobin, Int. J. Cosmetic Science, 2008).

When grey and white hair appear, they suggest age-related and genetically regulated exhaustion of the pigment-forming potential of each hair follicle. The aging of melanocytes can be associated ‘with damage mediated by reactive oxygen species to the nucleus and to mitochondrial DNA with subsequent buildup of mutations with age, besides an evident alteration in antioxidant mechanisms or in pro-apoptotic and anti-apoptotic factors in cells. Oxidative stress is generated by several factors, such as environmental factors and endogenous changes (radiations, inflammation, emotional stress) that accelerate the aging process.

Other data in the literature report that the continuous synthesis of melanin during the growth phases of hair (anagen) generates high levels of oxidative stress, and that melanocytes are particularly sensitive to aging induced by free radicals. In fact, it has been proven that the pigmentary unit of grey hair contains apoptotic melanocytes and also presents a high level of oxidative stress.

In some embodiments, the present disclosure is directed to a method for effecting changes in mammalian hair appearance, hair growth, hair pigmentation and hair follicle and hair shaft size, comprising topical application to the skin of a mammal an effective amount of a topically active composition comprising Withania somnifera derived exosome-like nanovesicles and/or exosomes or compositions thereof. In some embodiments all the methods and compositions herein are useful for the reduction of grey and white hair. In some embodiments all the methods and compositions herein are useful in preventing the formation of grey and white hair.

In some embodiments, the present disclosure is directed to a method for producing a melanogenetic action in the hair and to promote its pigmentation and pigmentation of the stem, comprising the step of administering to a subject in need thereof an effective amount of a Withania somnifera derived exosome-like nanovesicles and/or exosomes or compositions thereof.

In some embodiments, the present disclosure is directed to a method for increasing the production of melanocytes comprising administering to a subject in need thereof an effective amount of Withania somnifera derived exosome-like nanovesicles and/or exosomes or compositions thereof. In some embodiments the effective amount of Withania somnifera derived exosome-like nanovesicles and/or exosomes is between 1×10⁷ and 1×10¹² ASH-NVs/mL (i.e. Withania somnifera derived exosome-like nanovesicles and/or exosomes per mL). In some embodiments, the present disclosure is directed to a method for increasing the production of melanocytes comprising topical application to the skin of a mammal an effective amount of a topically active composition comprising Withania somnifera derived exosome-like nanovesicles and/or exosomes or compositions thereof.

In some embodiments all the methods and compositions for increasing the production of melanocytes are useful for the reduction of grey and white hair. In some embodiments all the methods and compositions for increasing the production of melanocytes are useful in preventing the formation of grey and white hair. In some embodiments all the methods and compositions for increasing the production of melanocytes are useful for effecting changes in mammalian hair appearance, hair growth, hair pigmentation and hair follicle and hair shaft size.

In some embodiments, the present disclosure is directed to a method for increasing melanin production in human primary melanocytes comprising administering to a subject in need thereof an effective amount of Withania somnifera derived exosome-like nanovesicles and/or exosomes or compositions thereof.

In some embodiments, the present disclosure is directed to a method for increasing melanin production in human primary melanocytes comprising topical application to the skin of a mammal an effective amount of a topically active composition comprising Withania somnifera derived exosome-like nanovesicles and/or exosomes or compositions thereof.

In some embodiments all the methods and compositions for increasing melanin production in human primary melanocytes are useful for the reduction of grey and white hair. In some embodiments all the methods and compositions for increasing melanin production in human primary melanocytes are useful in preventing the formation of grey and white hair. In some embodiments all the methods and compositions for increasing melanin production in human primary melanocytes are useful for effecting changes in mammalian hair appearance, hair growth, hair pigmentation and hair follicle and hair shaft size.

### Skin Treatment

In some embodiments, the Withania somnifera derived exosome-like nanovesicles and/or exosomes or compositions thereof described herein are useful for preventing, retarding, and/or treating uneven skin texture by regulating oily/shiny appearance. In some embodiments, the Withania somnifera derived exosome-like nanovesicles and/or exosomes or compositions thereof described herein are useful for regulating and/or reducing pore size appearance.

The disclosure further relates to methods for regulating the condition of mammalian keratinous tissue wherein the methods each comprise the step of topically applying to the keratinous tissue of a mammal needing such treatment, a safe and effective amount of the Withania somnifera derived exosome-like nanovesicles and/or exosomes or compositions thereof of the present disclosure. In some embodiments, the Withania somnifera derived exosome-like nanovesicles and/or exosomes or compositions thereof described herein are effective for the treatment of pruritus, chronic pruritus, skin roughening, skin dryness, scar therapy, scar lightening, reduction of pathological myofibroblasts.

In some embodiments, the Withania somnifera derived exosome-like nanovesicles and/or exosomes or compositions thereof described herein is useful to treat chronic pruritus is defined as an itch persisting for >6 weeks, which can be severe enough to interfere with lifestyle activities. 1 Pruritus can be a hallmark of many skin diseases as well as other noncutaneous diseases. Neuropathic, psychogenic, systemic, and dermatologic disorders constitute the majority of causes of pruritus.

In some embodiments, the Withania somnifera derived exosome-like nanovesicles and/or exosomes or compositions thereof described herein is useful to treat skin roughening, mainly due to dryness, is generally caused by damage to the intracellular lipids of the skin, which decreases the water-retention capacity of the stratum corneum.

In some embodiments, the Withania somnifera derived exosome-like nanovesicles and/or exosomes or compositions thereof described herein is useful to treat scar therapy via the mechanism of targeted killing the myofibroblasts.

The exosome-like nanovesicles and/or exosomes or composition may include disinfectants, antiseptics, or drug substances. Incorporation of one or more disinfectants or antiseptics is especially useful in those situations where it is important to inactivate the microorganisms which remain on the skin after normal cleansing. Incorporation of a drug substance in the composition may be useful for the prevention or treatment of various skin disorders or to deliver drug substances to the skin which are advantageously administered topically for percutaneous absorption.

In some embodiments, the Withania somnifera derived exosome-like nanovesicles and/or exosomes or compositions thereof described herein is useful to treat dermatosis. As used herein, term “dermatosis” should refer to the disease of skin, imbalance or defective, this includes but not limited to acne (including but not limited to acne vulgaris and acne rosacea), psoriasis, infect, flaw, pigmentation (include but not limited to inflammation after pigmentation (PIH)), hypopigmentation, hair growth imbalance (as the undue or unnecessary growth of alopecia and hair), pachylosis, skin is done, cutis laxa (include but not limited to skin-tightening and lack flexibility), wrinkle (including but not limited to microgroove and years stricture of vagina), blood vessel hyperplasia skin (including but not limited to skin dark stain), sebum generates imbalance (for example skin glow), the pore hypertrophy, excessively perspire (comprising hyperhidrosis), tatoo, erythra (comprising allergic rash and diaper rash), cicatrix, pain, scratch where it itches, burn, inflammation, wart, clavus, callus, edema, Rhus toxicodendron/poison lacquer rattan peel rash, skin carcinoma and insecticide, Aranea, biting of Serpentis and other animals.

In some embodiments, the Withania somnifera derived exosome-like nanovesicles and/or exosomes or compositions thereof described herein is useful to treat skin infections which includes but is not limited to acne, pustule, folliculitis, furunculosis, ecthyma, eczema, psoriasis, atoipc dermatitis, epidermolysis bullosa, ichthyosis, infected wound (ulcer that has for example infected, slight burns, incised wound, scratch, laceration, wound, tissue biopsy position, operative incision and sting place), herpes (for example cold sore) or other antibacterial or viral infection.

In some embodiments, the Withania somnifera derived exosome-like nanovesicles and/or exosomes or compositions thereof described herein is useful to treat wrinkles or skin lines which includes but is not limited to microgroove, deep wrinkle, laugh line, crows-feet, striae gravidarum, and liparitosis.

In some embodiments, the Withania somnifera derived exosome-like nanovesicles and/or exosomes or compositions thereof described herein is useful to treat variable color skin which includes but is not limited to pigmentation skin, hypopigmentation's skin, flaw skin, injury with blood-stasis and blood vessel hyperplasia skin.

In some embodiments, the Withania somnifera derived exosome-like nanovesicles and/or exosomes or composition thereof described herein is useful to treat pigmentation of the skin which includes but is not limited to pigmentation (PIH) and other variable color skin after freckle, senile plaque (Exposure to Sunlight freckle), sunshine speckle, chloasma, the sick Huang of face, pigmentation, the inflammation. An example of hypopigmentation includes but is not limited to vitiligo.

In some embodiments, the Withania somnifera derived exosome-like nanovesicles and/or exosomes or composition thereof described herein is useful to treat skin defects which includes but is not limited to the rash of pustule, blackhead, pimple, blackhead or other types relevant with acne. In some embodiments, the Withania somnifera derived exosome-like nanovesicles and/or exosomes or compositions thereof described herein is useful to treat dermopathic examples of cicatrix includes but is not limited to the cicatrix that caused by acne, operation, sting, burn, injured, wound and other wounds. In some embodiments, the composition described herein also can be used for the treatment of mucosal disease (for example oral cavity and vaginal mucosa disease). Include but is not limited to the example of mucosal disease periodontal disease, gingival, oropharynx cancer, Candida mycoderma infect, cause such as herpes of mouth such as cold sore and fever blister and as herpes simplex or other viral infection of the genital herpes of genital ulcer.

### Compositions

In some embodiments, the present disclosure is directed to compositions, optionally cosmetic compositions, comprising Withania somnifera derived exosome-like nanovesicles and/or exosomes. In some embodiments, the present disclosure is directed to a cosmetic composition comprising Withania somnifera derived exosome-like nanovesicles and/or exosomes and one or more additional ingredient(s) selected from water, a carrier, an emulsifier, a preservative, a thickener, an emollient, a coloring agent, a fragrance and a pH stabilizer.

In some embodiments, the Withania somnifera derived exosome-like nanovesicles and/or exosomes are within a composition. In some embodiments, the Withania somnifera derived exosome-like nanovesicles and/or exosomes composition further comprises a carrier. In some embodiments, the Withania somnifera derived exosome-like nanovesicles and/or exosomes composition is applied topically. In some embodiments, the Withania somnifera derived exosome-like nanovesicles and/or exosomes composition is topically applied to the scalp.

In some embodiments, the number of exosome-like nanovesicles and/or exosomes within the composition is between about 1×10⁷ per mL and about 1×10¹² per mL. In some embodiments, the number of exosome-like nanovesicles and/or exosomes within the composition is between about 1×10⁸ per mL and about 1×10¹² per mL. In some embodiments, the number of exosome-like nanovesicles and/or exosomes within the composition is between about 1×10⁹ per mL and about 1×10¹² per mL. In some embodiments, the number of exosome-like nanovesicles and/or exosomes within the composition is between about 1×10¹⁰ per mL and about 1×10¹² per mL. In some embodiments, the number of exosome-like nanovesicles and/or exosomes within the composition is between about 1×10⁷ per mL and about 1×10¹¹ per mL. In some embodiments, the number of exosome-like nanovesicles and/or exosomes within the composition is between about 1×10⁸ per mL and about 1×10¹¹ per mL. In some embodiments, the number of exosome-like nanovesicles and/or exosomes within the composition is between about 1×10⁹ per mL and about 1×10¹¹ per mL. In some embodiments, the number of exosome-like nanovesicles and/or exosomes within the composition is between about 1×10¹⁰ per mL and about 1×10¹¹ per mL. In some embodiments, the number of exosome-like nanovesicles and/or exosomes within the composition is between about 1×10⁸ per mL and about 1×10¹⁰ per mL. In some embodiments, the number of exosome-like nanovesicles and/or exosomes within the composition is between about 1×10⁹ per mL and about 1×10¹⁰ per mL. In some embodiments, the number of exosome-like nanovesicles and/or exosomes within the composition is about 1×10⁷ per mL. In some embodiments, the number of exosome-like nanovesicles and/or exosomes within the composition is about 1×10⁸ per mL. In some embodiments, the number of exosome-like nanovesicles and/or exosomes within the composition is about 1×10⁹ per mL. In some embodiments, the number of exosome-like nanovesicles and/or exosomes within the composition is about 1×10¹⁰ per mL. In some embodiments, the number of exosome-like nanovesicles and/or exosomes within the composition is about 1×10¹¹ per mL. In some embodiments, the number of exosome-like nanovesicles and/or exosomes within the composition is about 1×10¹² per mL.

In some embodiments, the Withania somnifera derived exosome-like nanovesicles and/or exosomes composition further comprises an alcohol-free carrier. In some embodiments, the Withania somnifera derived exosome-like nanovesicles and/or exosomes composition further comprises an alcohol-free carrier and is topically applied to the scalp. In some embodiments, the carrier base fluids that may be used in the Withania somnifera derived exosome-like nanovesicles and/or exosomes composition include any carrier fluid or combination of excipients suitable for use in cosmetic and/or medicinal applications. For example, the Withania somnifera derived exosome-like nanovesicles and/or exosomes composition may comprise an aqueous carrier base fluid. In an embodiment, the aqueous carrier base fluid comprises deionized water.

In some embodiments, the carrier base fluid may act as a solvent, carrier, diluent and/or dispersant for the constituents of the composition, and may allow for the uniform application of the constituents to the surface of the skin at an appropriate dilution, e.g., topical application. For example, carrier base fluids can be emulsions, lotions, creams, tonics, sprays, aerosols, and the like. The carrier base fluid may also facilitate penetration of the composition into the skin.

In some embodiments, the carrier base fluid comprises a lotion suitable for topical application. In such embodiment, the lotion may comprise carbomer, water, glycerin, isopropyl myristate, mineral oil, stearic acid, glycol stearate, cetyl alcohol, dimethicone, preservatives, triethanolamine, and the like, or combinations thereof.

In some embodiments, the carrier base fluid comprises a gel suitable for topical application. In such embodiment, the gel may comprise water, carbomer, glycerin, propylene glycol, preservatives, and the like, or combinations thereof.

The carrier base fluid may be present in an amount of from about 1 wt. % to about 99.99 wt. % based on the total weight of the Withania somnifera derived exosome-like nanovesicles and/or exosomes. Alternatively, the carrier base fluid may comprise the balance of the Withania somnifera derived exosome-like nanovesicles and/or exosomes composition after considering the amount of the other components used.

In some embodiments, the Withania somnifera derived exosome-like nanovesicles and/or exosomes may be soluble or insoluble in the carrier base fluid. In an embodiment, the Withania somnifera derived exosome-like nanovesicles and/or exosomes are soluble in the carrier base fluid, and the carrier base fluid acts as a solvent. In another embodiment, one or more of the ingredients used in the Withania somnifera derived exosome-like nanovesicles and/or exosomes composition may be solubilized in a solubilizer prior to mixing in the carrier base fluid, such that these ingredients become soluble in the carrier base fluid. Nonlimiting examples of solubilizers suitable for use in the present disclosure include water, glycerin (e.g., vegetable glycerin), various esters, polyethylene glycol (PEG), derivatives thereof, or combinations thereof.

In some embodiments, the Withania somnifera derived exosome-like nanovesicles and/or exosomes composition may further comprise inactive ingredients, such as surfactants, co-solvents, and excipients or fillers (e.g., solid, semi-solid, liquid, etc.); emollients; delivery enhancers; circulation enhancers; antimicrobial agents; anti-inflammatory agents; foaming agents; carriers; diluents; binding agents (e.g., dextran); thickening agents; gelling agents; vitamins, retinoids, and retinols (e.g., vitamin B3, vitamin A, etc.); pigments; fragrances; sunscreens and sunblocks; anti-oxidants and radical scavengers (e.g., tocopheryl acetate or vitamin E acetate); organic hydroxy acids; exfoliants; skin conditioners (e.g., ethylhexylglycerin, hydrolyzed soy protein, glycol distearate, cyclopentasiloxane, quaternium-79 hydrolyzed keratin, propylene glycol, etc.); moisturizers; humectants (e.g., hydrolyzed soy protein, propylene glycol, etc.); ceramides, pseudoceramides; phospholipids, sphingolipids, cholesterol, glucosamine; pharmaceutically acceptable penetrating agents (e.g., n-decylmethyl sulfoxide, lecithin organogels, tyrosine, lysine, etc.); preservatives (e.g., phenoxyethanol, benzoic acid, dehydroacetic acid, polyaminopropyl biguanide, DMDM hydantoin or 1,3-bis(hydroxymethyl)-5,5-dimethylimidazolidine-2,4-dione, iodopropynyl butylcarbamate, iodopropynyl butylcarbonate, stearalkonium chloride, etc.); amino acids such as proline; pyrrolidone carboxylic acid, its derivatives and salts; saccharide isomerate; panthenol (i.e., provitamin of B5); buffers together with a base such as triethanolamine or sodium hydroxide; waxes, such as beeswax, ozokerite wax, paraffin wax; plant extracts, obtained from plants such as Aloe vera leaf, cornflower, witch hazel, elderflower, green tea (e.g., Camellia sinensis) leaf, grape (e.g., Vitis vinifera) seed, jojoba (e.g., Simmondsia chinensis) seed, tea tree (e.g., Melaleuca alternifolia) leaf, rosemary (e.g., Rosmarinus officinalis), henna, sunflower (e.g., Helianthus annuus) seed, wild soybean (e.g., Glycine soja), argan tree kernel or argan, cucumber, shiso, etc.; or combinations thereof. As will be appreciated by one of skill in the art viewing this disclosure, the selection and amount of optional ingredients may be varied.

In some embodiments, the present disclosure is directed to a composition comprising Withania somnifera derived exosome-like nanovesicles and/or exosomes and one or more of water, glycerin, Camellia sinensis(green tea) leaf extract, glycine, Larix europaea wood extract, sodium metabisulfite, zinc chloride, Pisum sativum (pea) sprout extract, alcohol, Olea europaea (olive) leaf extract, Curcuma longa(turmeric) root extract, Equisetum arvense (horsetail) extract, Hippophae rhamnoides (sea buckthorn) fruit oil, Laminaria saccharina (neptune kelp) extract, Lepidium meyenii (maca) root extract, Melaleuca alternifolia (tea tree) leaf oil, Moringa oleifera (moringa) leaf extract, Panax ginseng(ginseng) root extract, DL-panthenol, L-theanine, Melatonin, Niacinamide, sodium dehydroacetate, sodium hyaluronate, and phytic acid.

In some embodiments, the present disclosure is directed to a composition comprising Withania somnifera derived exosome-like nanovesicles and/or exosomes, glycerin, Camellia sinensis(green tea) leaf extract, glycine, Larix europaea wood extract, sodium metabisulfite, zinc chloride, Pisum sativum (pea) sprout extract, alcohol, Olea europaea (olive) leaf extract, Curcuma longa(turmeric) root extract, Equisetum arvense (horsetail) extract, Hippophae rhamnoides (sea buckthorn) fruit oil, Laminaria saccharina (neptune kelp) extract, Lepidium meyenii (maca) root extract, Melaleuca alternifolia (tea tree) leaf oil, Moringa oleifera (moringa) leaf extract, Panax ginseng(ginseng) root extract, DL-panthenol, L-theanine, Melatonin, Niacinamide, sodium dehydroacetate, sodium hyaluronate, and phytic acid.

In some embodiments, the present disclosure is directed to a composition comprising Withania somnifera derived exosome-like nanovesicles and/or exosomes and one or more of water, glycerin, Melaleuca alternifolia leaf water propanediol, 1,2-hexanediol, panthenol, niacinamide, hydroxyethylcellulose, Lepidium meyenii, root extract, maltodextrin, caprylhydroxamic acid, Hippophae rhamnoides fruit extract, Equisetum arvense extract, Laminaria saccharina extract, Chondrus crispus extract, sodium metabisulfite, alcohol, phospholipids, arginine, lactic acid, melatonin, potassium sorbate, lactobacillus ferment, Pisum sativum extract, and/or phosphate buffered saline.

In some embodiments, the present disclosure is directed to a composition comprising Withania somnifera derived exosome-like nanovesicles and/or exosomes, water, glycerin, Melaleuca alternifolia leaf water propanediol, 1,2-hexanediol, panthenol, niacinamide, hydroxyethylcellulose, Lepidium meyenii, root extract, maltodextrin, caprylhydroxamic acid, Hippophae rhamnoides fruit extract, Equisetum arvense extract, Laminaria saccharina extract, Chondrus crispus extract, sodium metabisulfite, alcohol, phospholipids, arginine, lactic acid, melatonin, potassium sorbate, lactobacillus ferment, Pisum sativum extract, and/or phosphate buffered saline.

In some embodiments, the present disclosure is directed to a composition comprising Withania somnifera derived exosome-like nanovesicles and/or exosomes and one or more of water, glycerin, Melaleuca alternifolia leaf water, propanediol, butylene glycol, caffeine, 1,2-hexanediol, niacinamide, hydroxyethylcellulose, panthenol, Lepidium meyenii root extract, maltodextrin, caprylhydroxamic acid, Chondrus crispus extract, Hippophae rhamnoides fruit extract, Laminaria saccharina (neptune kelp) extract, alcohol, phospholipids, sodium metabisulfite, arginine, lactic acid, melatonin, potassium sorbate, lactobacillus ferment, Pisum sativum extract, phosphate buffered saline, and/or Panax ginseng root extract.

In some embodiments, the present disclosure is directed to a composition comprising Withania somnifera derived exosome-like nanovesicles and/or exosomes, water, glycerin, Melaleuca alternifolia leaf water, propanediol, butylene glycol, caffeine, 1,2-hexanediol, niacinamide, hydroxyethylcellulose, panthenol, Lepidium meyenii root extract, maltodextrin, caprylhydroxamic acid, Chondrus crispus extract, Hippophae rhamnoides fruit extract, Laminaria saccharina (neptune kelp) extract, alcohol, phospholipids, sodium metabisulfite, arginine, lactic acid, melatonin, potassium sorbate, lactobacillus ferment, Pisum sativum extract, phosphate buffered saline, and/or Panax ginseng root extract.

In some embodiments, the composition comprises water. In some embodiments, the composition comprises glycerin. In some embodiments, the composition comprises Camellia sinensis (green tea) leaf extract. In some embodiments, the composition comprises glycine. In some embodiments, the composition comprises Larix europaea wood extract. In some embodiments, the composition comprises sodium metabisulfite. In some embodiments, the composition comprises zinc chloride. In some embodiments, the composition comprises Pisum sativum (pea) sprout extract. In some embodiments, the composition comprises an alcohol. In some embodiments, the composition comprises Olea europaea (olive) leaf extract. In some embodiments, the composition comprises Curcuma longa (turmeric) root extract. In some embodiments, the composition comprises Equisetum arvense (horsetail) extract. In some embodiments, the composition comprises Hippophae rhamnoides (sea buckthorn) fruit oil. In some embodiments, the composition comprises Laminaria saccharina (neptune kelp) extract. In some embodiments, the composition comprises Lepidium meyenii (maca) root extract. In some embodiments, the composition comprises Melaleuca alternifolia (tea tree) leaf oil. In some embodiments, the composition comprises Moringa oleifera (moringa) leaf extract. In some embodiments, the composition comprises Panax ginseng (ginseng) root extract. In some embodiments, the composition comprises DL-panthenol. In some embodiments, the composition comprises L-theanine. In some embodiments, the composition comprises melatonin. In some embodiments, the composition comprises niacinamide. In some embodiments, the composition comprises sodium dehydroacetate. In some embodiments, the composition comprises sodium hyaluronate. In some embodiments, the composition comprises phytic acid. In some embodiments, the composition comprises Melaleuca alternifolia leaf water. In some embodiments, the composition comprises propanediol. In some embodiments, the composition comprises 1,2-hexanediol. In some embodiments, the composition comprises Panthenol. In some embodiments, the composition comprises hydroxyethylcellulose. In some embodiments, the composition comprises Lepidium meyenii root extract. In some embodiments, the composition comprises maltodextrin. In some embodiments, the composition comprises caprylhydroxamic acid. In some embodiments, the composition comprises Hippophae rhamnoides fruit extract. In some embodiments, the composition comprises Equisetum arvense extract. In some embodiments, the composition comprises Laminaria saccharina extract. In some embodiments, the composition comprises Chondrus crispus extract. In some embodiments, the composition comprises sodium metabisulfite. In some embodiments, the composition comprises ethyl alcohol. In some embodiments, the composition comprises phospholipids. In some embodiments, the composition comprises arginine. In some embodiments, the composition comprises lactic acid. In some embodiments, the composition comprises potassium sorbate. In some embodiments, the composition comprises lactobacillus ferment. In some embodiments, the composition comprises Pisum sativum extract. In some embodiments, the composition comprises phosphate buffered saline. In some embodiments, the composition comprises butylene glycol. In some embodiments, the composition comprises caffeine. In some embodiments, the composition comprises Chondrus crispus extract. In some embodiments, the composition comprises Laminaria saccharina (neptune kelp) extract. In some embodiments, the composition comprises Panax ginseng root extract.

In some embodiments, the composition comprises the Withania somnifera-extracted exosome-like nanovesicles or exosomes in an amount of about 0.01% to 10% by weight of the composition. In some embodiments, the composition comprises the Withania somnifera-extracted exosome-like nanovesicles or exosomes in an amount of about 0.01% to 1% by weight of the composition. In some embodiments, the composition comprises the Withania somnifera-extracted exosome-like nanovesicles or exosomes in an amount of about 0.01% to 2% by weight of the composition. In some embodiments, the composition comprises the Withania somnifera-extracted exosome-like nanovesicles or exosomes in an amount of about 0.01% to 3% by weight of the composition. In some embodiments, the composition comprises the Withania somnifera-extracted exosome-like nanovesicles or exosomes in an amount of about 0.01% to 4% by weight of the composition. In some embodiments, the composition comprises the Withania somnifera-extracted exosome-like nanovesicles or exosomes in an amount of about 0.01% to 5% by weight of the composition. In some embodiments, the composition as described in any of the above comprises 0.1% to 1% Withania somnifera exosome-like nanovesicles and/or exosomes. In some embodiments, the composition as described in any of the above comprises 0.1% to 2% Withania somnifera exosome-like nanovesicles and/or exosomes. In some embodiments, the composition as described in any of the above comprises 0.1% to 3% Withania somnifera exosome-like nanovesicles and/or exosomes. In some embodiments, the composition as described in any of the above comprises 0.1% to 4% Withania somnifera exosome-like nanovesicles and/or exosomes. In some embodiments, the composition as described in any embodiment herein comprises 0.1% to 5% Withania somnifera exosome-like nanovesicles and/or exosomes. In some embodiments, the composition as described in any embodiment herein comprises 0.1% to 10% Withania somnifera exosome-like nanovesicles and/or exosomes. In some embodiments, the composition as described in any of the above comprises about 0.3% to about 1% Withania somnifera exosome-like nanovesicles and/or exosomes.

### Combinations

In some embodiments, the Withania somnifera derived exosome-like nanovesicles and/or exosomes are administered in combination with one or more additional active ingredient. In some embodiments, the Withania somnifera derived exosome-like nanovesicles and/or exosomes are administered in combination with a human derived exosome. In some embodiments, the Withania somnifera derived exosome-like nanovesicles and/or exosomes are administered in combination with another plant derived exosome-like nanovesicle and/or exosome. In some embodiments, the Withania somnifera derived exosome-like nanovesicles and/or exosomes are administered in combination with an aloe derived exosome-like nanovesicle and/or exosome. In some embodiments, the Withania somnifera derived exosome-like nanovesicles and/or exosomes are administered in combination with both a human derived exosome and an aloe derived exosome-like nanovesicle and/or exosome.

In some embodiments, the combination of active ingredients are in the same composition. In some embodiments, the combination of active ingredients are administered as separate compositions. In some embodiments, the Withania somnifera derived exosome-like nanovesicles and/or exosomes are administered in combination with both a human derived exosome and an aloe derived exosome-like nanovesicle and/or exosome, wherein the ingredients are in separate compositions. In some embodiments, the Withania somnifera derived exosome-like nanovesicles and/or exosomes are administered in combination with both a human derived exosome and an aloe derived exosome-like nanovesicle and/or exosome, wherein the ingredients as a part of the same composition.

In some embodiments, the Withania somnifera derived exosome-like nanovesicles and/or exosomes are administered in combination with a human derived exosome, wherein the ingredients as a part of the same composition. In some embodiments, the Withania somnifera derived exosome-like nanovesicles and/or exosomes are administered in combination with a human derived exosome, wherein the ingredients are in separate compositions.

In some embodiments, the Withania somnifera derived exosome-like nanovesicles and/or exosomes are administered in combination with an aloe derived exosome-like nanovesicle and/or exosome, wherein the ingredients as a part of the same composition. In some embodiments, the Withania somnifera derived exosome-like nanovesicles and/or exosomes are administered in combination with an aloe derived exosome-like nanovesicle and/or exosome, wherein the ingredients are in separate compositions.

### Administration

The Withania somnifera derived exosome-like nanovesicles and/or exosomes composition may be applied to skin and hair using any suitable treatment regime. The Withania somnifera derived exosome-like nanovesicles and/or exosomes composition may be applied at least once a week, such as at least every two days, or at least once each day. For example, application may be twice per day.

In general, treatment using the Withania somnifera derived exosome-like nanovesicles and/or exosomes composition described here may be continued indefinitely. Alternatively, the treatment may be repeated only for a limited period, e.g. several weeks or months. Treatment may then be repeated for a similar period at a later date.

Most commonly, the area of the skin to which the composition is applied will be the scalp, i.e., the composition will be used to combat hair loss on the user's head. Other areas may be suitable for application, for example to promote the growth of eyebrow hair, or eyelashes. In some embodiments, in addition to treating or preventing hair loss and/or promoting the growth of the hair, the methods and compositions described here may also improve the appearance of hairs to which the composition is applied, e.g. by thickening the hair and improving the lustre, color (less grey, less white) condition and manageability of the hair.

In some embodiments, a method for treating hair loss comprises topical application of the exosome composition on the scalp or any other body area where hair growth or regrowth is desirable. The Withania somnifera derived exosome-like nanovesicles and/or exosomes may be useful for treating hair loss by preventing or slowing hair loss and/or stimulating or increasing hair growth or regrowth. The compositions comprising Withania somnifera derived exosome-like nanovesicles and/or exosomes described herein may be useful in a wide variety of finished products, including pharmaceutical products and cosmetic products. The exosomes may be prepared, packaged, and labeled for modulation of hair growth or regrowth, and for diminishing the hair loss process.

In some embodiments, the Withania somnifera derived exosome-like nanovesicles and/or exosomes disclosed herein may be topically administered in the form of a solution, aqueous solution, gel, lotion, cream, ointment, oil-in-water emulsion, water-in-oil emulsion, stick, spray, aerosol, paste, mousse, tonic, liposome or other cosmetically and topically suitable form. In an embodiment, the Withania somnifera derived exosome-like nanovesicles and/or exosomes or composition thereof may be topically applied to an area to be treated, for example the scalp in humans, by dropper, spraying, dabbing, swabbing, rubbing, or combinations thereof.

In some embodiments, the Withania somnifera derived exosome-like nanovesicles and/or exosomes may be topically applied in the form of a scalp stimulator foam. In another embodiment, the Withania somnifera derived exosome-like nanovesicles and/or exosomes may be topically dispersed on the scalp in an aerosol form such as in a chlorofluorocarbon solvent, for delivery in spray form. The spray form may present some advantages including high loading, enhanced drug uptake, convenient application, and less matting the hair in the region of application. In such embodiments, the exosomes may remain on the scalp for a period of time of about 1 week, alternatively about 1 day, alternatively about 12 h, alternatively about 4 h, alternatively about 1 h, alternatively about 30 min, alternatively about 5 min, or alternatively about 1 min. The exosomes may be removed at any desired point in time by washing and/or rinsing the scalp.

In some embodiments, the Withania somnifera derived exosome-like nanovesicles and/or exosomes may be topically applied in the form of a shampoo, conditioner, or any other suitable hair care product formulation, or combinations thereof. In such embodiment, the shampoo or conditioner may be rinsed after application, for example, immediately after the application, alternatively after a period of time of about 5 s, alternatively about 30 s, alternatively about 1 min, alternatively about 5 min., alternatively about 30 min, alternatively about 1 h, alternatively about 4 h, alternatively about 12 h, or alternatively about 24 h. In an embodiment, the conditioner may be the “leave-in” type: conditioner, e.g., the conditioner may be left on the scalp without rinsing until the next scalp washing. In an embodiment, more than one form of Withania somnifera derived exosome-like nanovesicles and/or exosomes may be applied to the hair, for example, in one treatment session, alternatively in different treatment sessions, the Withania somnifera derived exosome-like nanovesicles and/or exosomes may be topically applied to the scalp as a shampoo, conditioner, scalp stimulator foam, or combinations thereof.

In some embodiments, the Withania somnifera derived exosome-like nanovesicles and/or exosomes may be topically administered at least on a daily, a twice daily, or a three times daily, basis for a period of time sufficient to bring about the desired level of improvement in modulation of hair growth or regrowth. For example, a user may topically administer the Withania somnifera derived exosome-like nanovesicles and/or exosomes directly to a balding area or other area where increased hair growth is desired by gently massaging the composition of the present disclosure into the desired area. This process may be repeated later the same day. In an embodiment, the Withania somnifera derived exosome-like nanovesicles and/or exosomes may be left on the scalp or other area where increased hair growth is desired between applications occurring on the same day or on different days. As will be appreciated by one skilled in the art with the help of this disclosure, when the Withania somnifera derived exosome-like nanovesicles and/or exosomes may be topically applied/administered periodically on a routine basis prior to, during, and subsequent to modulation of hair growth or regrowth. Generally, the Withania somnifera derived exosome-like nanovesicles and/or exosomes may be topically administered on a daily basis, although more frequent applications also may be used.

In some embodiments, the application exosomes may continue for any suitable period of time. For example, within a few weeks to a few months of the initial application, a user may notice a reduction in hair loss and/or an increase in hair growth or regrowth. It should be appreciated that the frequency with which the Withania somnifera derived exosome-like nanovesicles and/or exosomes should be applied will vary depending on the desired effect. In some embodiments, the degree of cosmetic enhancement might vary directly with the total amount of Withania somnifera derived exosome-like nanovesicles and/or exosomes used.

In some embodiments, disclosed herein is a method of treating a skin or a hair condition comprising administering a composition to dermal papilla cells of a subject, wherein the composition comprises hinoki oil, red clover extract, and a peptide; and increasing a growth factor from the dermal papilla cells of the subject in response to administering the composition.

In some embodiments, disclosed herein is a method of treating a skin or a hair condition comprising administering a composition to dermal papilla cells of a subject, wherein the composition comprises hinoki oil, red clover extract, and a peptide; and increasing secreted leukemia inhibitory factor (LIF), placental growth factor 1 (PLGF-1), basic fibroblast growth factor (FGF-2), vascular endothelial growth factor A (VEGF-A), or any combination thereof, from the dermal papilla cells of the subject in response to administering the composition.

As will be appreciated by those of skill in the art with the help of this disclosure, other methods may be used to topically apply/administer the exosomes described herein.

In an embodiment, a composition for the treatment of hair loss such as Withania somnifera derived exosome-like nanovesicles and/or exosomes may be advantageously used to diminish hair loss and/or promote hair growth and/or regrowth. For example, as disclosed herein, a composition for the treatment of hair loss such as the Withania somnifera derived exosome-like nanovesicles and/or exosomes may diminish and/or stop hair loss in a time period of from about 7 days to about 80 days, alternatively from about 10 days to about 28 days, or alternatively from about 14 days to about 21 days.

While not intending to be limited by theory, in some embodiments, the Withania somnifera derived exosome-like nanovesicles and/or exosomes and compositions thereof may advantageously regrow hair in a time period of from about 4 weeks to about 52 weeks, alternatively from about 6 weeks to about 26 weeks, or alternatively from about 8 weeks to about 12 weeks.

In some embodiments, the Withania somnifera derived exosome-like nanovesicles and/or exosomes and compositions thereof advantageously diminish and/or stop the hair loss on the scalp when Withania somnifera derived exosome-like nanovesicles and/or exosomes and compositions thereof are topically applied to the scalp.

In an embodiment, the Withania somnifera derived exosome-like nanovesicles and/or exosomes and compositions thereof may advantageously promote hair growth from dormant and/or injured hair follicles, e.g., the Withania somnifera derived exosome-like nanovesicles and/or exosomes and compositions thereof may have a rejuvenating effect on the hair follicles. Additional advantages of the Withania somnifera derived exosome-like nanovesicles and/or exosomes and compositions thereof and methods of using same may be apparent to one of skill in the art viewing this disclosure.

Dosage of the Withania somnifera derived exosome-like nanovesicles and/or exosomes composition of the disclosure is dependent upon many factors including, but not limited to, the severity of the hair loss, the subject's age, general health and individual response to the compositions of the disclosure. Accordingly, dosages of the compositions can vary and be readily adjusted, depending on each subject's response.

In some embodiments, the present disclosure is directed to an article of manufacture or kit containing a topical dosage form prepared from Withania somnifera derived exosome-like nanovesicles and/or exosomes, packaged for retail distribution, in association with instructions advising the consumer how to use the product to promote hair growth.

The exosomes may be used to manufacture preparations to promote hair growth in other mammals besides humans. For example, the exosomes may be used with farm animals such as sheep, in which fur (hair) growth would exhibit an economic benefit. The exosomes may also be used to stimulate hair growth in companion animals such as dogs, cats, gerbils, etc. The dosages required to obtain this effect will fit within the guidelines described above. Likewise, the exosomes may be administered using formulations typically used for veterinary applications, taking into account the type of animal being treated. Other applications of the exosomes to promote hair growth will become readily apparent to one skilled in the art based upon the disclosure of this application and should be considered to be encompassed by the claims.

### Routes of Administration

The Withania somnifera derived exosome-like nanovesicles and/or exosomes of the present disclosure, or compositions thereof, can be administered orally, ingested, transdermally, subcutaneously, intramuscularly, and intravenously. In some embodiments, the Withania somnifera derived exosome-like nanovesicles and/or exosomes is administered orally or ingested. In some embodiments, the Withania somnifera derived exosome-like nanovesicles and/or exosomes is administered topically. In some embodiments, the Withania somnifera derived exosome-like nanovesicles and/or exosomes is administered topically to the scalp. One skilled in the art will recognize the advantages of certain routes of administration.

Dosage forms for the topical or transdermal administration of the Withania somnifera derived exosome-like nanovesicles and/or exosomes of this disclosure include powders, sprays, ointments, pastes, creams, lotions, gels, solutions, patches and inhalants. In some embodiments, the Withania somnifera derived exosome-like nanovesicles and/or exosomes is mixed under sterile conditions with an acceptable carrier, and with any preservatives, buffers or propellants that are required.

For administration by inhalation, the Withania somnifera derived exosome-like nanovesicles and/or exosomes are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the Withania somnifera derived exosome-like nanovesicles and/or exosomes are formulated into ointments, salves, gels, or creams as generally known in the art.

A composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water, saline solution, fixed oils, ethyl alcohol, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

### Methods of Production

In some embodiments, a composition comprising Withania somnifera derived exosome-like nanovesicles and/or exosomes for treating a hair follicle is provided, the composition comprising: i) an effective amount of isolated the Withania somnifera derived exosome-like nanovesicles and/or exosomes; and ii) a carrier, wherein the isolated the Withania somnifera derived exosome-like nanovesicles and/or exosomes are produced by a process comprising: (a) growing a Withania somnifera plant; and (h) isolating the Withania somnifera exosome-like nanovesicles and/or exosomes from one or more of the Withania somnifera stem, root, seeds, leaf, or fruit.

In some embodiments, a composition comprising Withania somnifera derived exosome-like nanovesicles and/or exosomes for treating a hair follicle is provided, the composition comprising: i) an effective amount of isolated the Withania somnifera derived exosome-like nanovesicles and/or exosomes; and ii) a carrier, wherein the isolated the Withania somnifera derived exosome-like nanovesicles and/or exosomes are produced by a process comprising: (a) growing a Withania somnifera plant, wherein the growth condition includes a step of heat shocking plant by increasing the temperature from a range of about 20° C. to about 30° C. to about 33° C. to about 37° C. for about 1 hour to about 3 hours, and/or to about 40° C. to about 45° C. for about 1 hour to about 3 hours, and wherein one or more of the Withania somnifera stem, root, seeds, leaf or fruit contains the Withania somnifera exosome-like nanovesicles and/or exosomes having the increased levels of heat shock stress-response molecules; and (h) isolating the Withania somnifera exosome-like nanovesicles and/or exosomes having increased levels of heat shock stress-response molecules.

In some embodiments, the present disclosure is directed to a composition comprising Withania somnifera derived exosome-like nanovesicles and/or exosomes for treating a hair follicle, the composition comprising: i) an effective amount of isolated/extracted Withania somnifera derived exosome-like nanovesicles and/or exosomes; and ii) a carrier, Wherein the isolated the Withania somnifera derived exosome-like nanovesicles and/or exosomes are produced by a process comprising: (a) growing a Withania somnifera plant, wherein the growth condition includes a step of heat shocking plant by increasing the temperature from a range of about 20° C. to about 30° C., to about 33° C. to about 37° C. for about 1 hour to about 3 hours, and/or to about 40° C. to about 45° C. for about 1 hour to about 3 hours, and wherein the Withania somnifera root contains the Withania somnifera exosome-like nanovesicles and/or exosomes having the increased levels of heat shock stress-response molecules; and (b) isolating the Withania somnifera exosomes having increased levels of heat shock stress-response molecules.

In some embodiments, the Withania somnifera is dried. In some embodiments, the Withania somnifera is dried after reaching maturity. In some embodiments, the Withania somnifera seeds are dried. In some embodiments, the Withania somnifera seeds are dried and subsequently rehydrated before extracting the exosomes.

### EXAMPLES

While the present disclosure has been described in terms of exemplary aspects, those skilled in the art will recognize that the present disclosure can be practiced with modifications in the spirit and scope of the appended claims. These examples and embodiments given above are merely illustrative and are not meant to be an exhaustive list of all possible designs, aspects, applications or modifications of the present disclosure.

### Example 1: Ashwagandha Exosome Protein Analysis and Heat Shocked Ashwagandha (Withania somnifera) Extraction Protocol

Different parts of the Ashwagandha plant including but not limited to the root, central stem, leaf stem, leaf and fruit were used for extraction. This extraction procedure has never been completed with the Ashwagandha plant and it is also the first time that the Ashwaghanda plant exosomes have produced and characterized. The parts of the Ashwagandha plant for exosome extraction are the root, the stem, the leaf, the fruit, the seed and then the heat shocked version of the leaf and the stem.

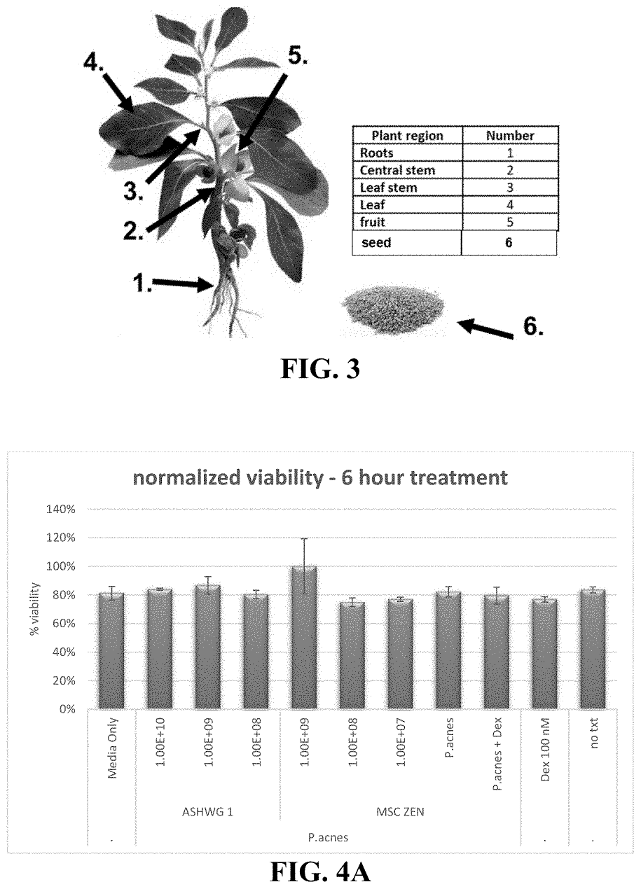

Ashwagandha (Withania somnifera) plants were harvested after 2 weeks and were dissected into 5 parts, according to FIG. 3. A small portion of each sample was retained for RNA extraction and western blot analysis.

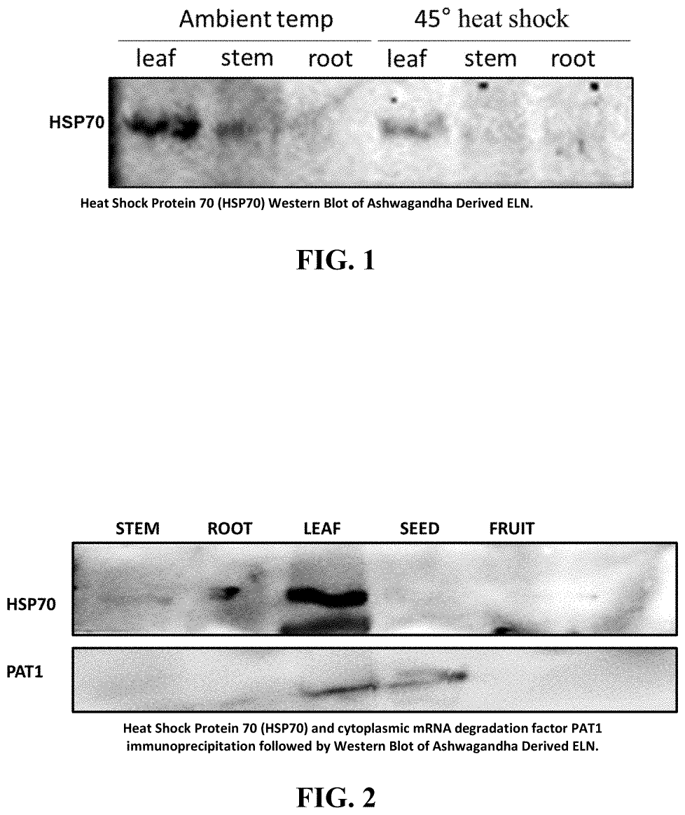

Maturing Ashwagandha plants were maintained at ambient temperature or exposed to 45° C. for 3 hours of heat shock. The leaves, stems and roots were harvested. After homogenization, exosomes were isolated from each sample and quantified for particle number and protein content. 20 ugs of protein were loaded onto a 4-12% gradient gel for electrophoresis and transferred to PVDF membrane. Blots were probed with antibodies from PhytoAB: HSP70 antibody (PHY0167), 1:1000 and SYP41/43 (PHY1010S), 1:1000. The results shown in FIG. 1 show that the HSP70 antibody recognized a band migrating around 75 KDa. HSP70 is most abundant in the leaves with very low detection in the roots at ambient temperatures. After heat shock, HSP70 quantity is lower in the leaves and the stem but appears to be increased in the roots. SYP proteins were not detected in exosomes derived from ambient temperature plant parts. Interestingly, heat shock loaded exosomes isolated from the leaves and stem with SYP proteins.

Exosomes were prepared as described and 20 ugs of protein were loaded onto a 4-12% gradient gel for electrophoresis and transferred to PVDF membrane. Blots were probed with antibodies from ABCAM: PAT1a antibody (ab124257), 1:1000 and PAT1b (ab139556), 1:1000. The results in FIG. 2 show that PAT1b was detected in leaves and stem of ambient temperature preps. Heat shock appears to reduce the quantity of PAT1b in the leaves and having no effect of PAT1b loading in stem and roots. PAT1a antibody showed no detection in any sample. Signal could be increased with more input sample and/or lower antibody dilution (1:100).

### Homogenization Protocol for Ashwagandha Derived Exosomes

Plant tissue samples were added to 15 ml Precellys homogenization bead vial containing 1.4 mm and 2.8 mm ceramic beads (CKmix50). Sterile PBS (7 mL) was added followed by the homogenization protocol. Pulse samples at 8000 rpm for 20 s. Remove samples and place on dry ice for 45 s. Repeat 3 more times. Pulse samples at 10000 rpm for 20 s. Remove samples and place on dry ice for 45 s. Remove contents to 50 mL conical tubes. Rinse beads with 7 ml of PBS. Repeat 3 times. Volume sample to ˜40 mL. Mix and place at −80° C. until ready for exosome purification. Isolate exosome using centrifugation. Quantify yield. Purify RNA from exosomes. Make cDNA. Quantify gene expression. Protein analysis was performed by western blot, flow cytometry, and immunostaining.

Each sample was filtered through a 0.22 um filter before centrifugation at 100,000×g for 2 hr. Root, and stem exosomes pellets were resuspended in 0.5 mL DPBS. Leaf pellet required resuspension in 5 mL DPBS and filtration through a 0.22 uM filter and re-centrifugation at 100,000×g for 2 hr. Before resuspending exosomes pellet in 0.5 mL DPBS exosomes were characterized using a Thermo NanoDrop spectrophotometer for protein determination and approximate RNA concentration by direct absorbance; exosomes were not lysed, stained, or RNA extracted prior to measurements. Particle diameter and concentration were assessed by tunable resistive pulse sensing (TRPS; qNano, Izon Science Ltd) using a NP150 nanopore membrane at a 47 mm stretch. The concentration of particles was standardized using multi-pressure calibration with 110 nm carboxylated polystyrene beads at a concentration of 1.2×10¹³ particles/mL

TABLE 1
Initial Isolation of Exosomes from Ashwagandha

| Sample | Protein mg/ml | RNA ng/ul | 260/280 | 260/230 |
|---|---|---|---|---|
| Ashwagandha | 0.47 | 21.1 | 1.17 | 0.266 |
| Leaves | | | | |
| Ashwagandha | 3.31 | 102.1 | 1.34 | 0.39 |
| Stem | | | | |
| Ashwagandha | 2.05 | 91.5 | 1.42 | 0.44 |
| Root | | | | |
| | Diameter Mean | Diameter Mode | Concentration | Particles/gram |
| Sample | (nm) | (nm) | (particle/ml) | of tissue |
| Ashwagandha | 117 | 107 | 1.89 × 10¹⁰ | 6.64 × 109 |
| Leaves | | | | |
| Ashwagandha | 133 | 114 | 1.87 × 10¹¹ | 4.56 × 10¹⁰ |
| Stem | | | | |
| Ashwagandha | 126 | 107 | 2.19 × 10¹¹ | 6.64 × 10¹⁰ |
| Root | | | | |

Stressors lead to an increase in the number of exosomes released and the Ashwagandha plant were heat stressed before extracting the exosomes. This results in the exosomes being “primed’ to conduct therapeutic effects, and in some instances, against chronically elevated levels of cortisol.

The heat shock protocol started with priming the Ashwagandha plant at 35° C. for 2 hours. Followed by heat shocking the plant at 45° C. for 3 hours followed by a 24 hr recovery period at room temperature. Then the samples were homogenized with Precellys following this 24 hr recovery period at room temperature. Then the samples were diluted with PBS and frozen at −80° C. until ready for centrifugation. The last step involved isolating the exosome using centrifugation and subsequently quantifying the yield. This was then followed by the homogenization protocol for the head shocked extracts. The sample was added to a 15 ml Precellys homogenization bead vial containing 1.4 mm and 2.8 mm ceramic beads (CKmix50). 7 mL of sterile PBS was added and then the homogenization protocol was run. The samples were pulsed at 8000 rpm for 20 s and then removed and placed on dry ice for 45 s. this process was repeated 3 more times. Subsequently the samples were pulse at 10,000 rpm for 20 s.

The samples were removed and placed on dry ice for 45 s. Next the contents were rinsed with beads with 7 mL of PBS which was repeated three times. The volume of the sample equated to around ˜40 mL. The samples were placed at −80° C. until ready for exosome purification. The last steps were the exosome isolation and purification before conducting a standard protein analysis such as western blot, flow cytometry, immunostaining.

The results of exosome extraction process for both the heat stressed Ashwagandha exosomes and the non-heat stressed exosomes are depicted in Table 2.

TABLE 2
Characterization of Ashwagandha Exosome Heat Stressed and Non-Heat Stressed

| | | | | | Total# | | |
|---|---|---|---|---|---|---|---|
| | Diameter | Diameter | Concentration | Total | particles | Particles/uL | Fraction |
| Sample | Mean (nm) | Mode (nm) | (particle/mL) | Volume (uL) | 10¹⁰ | (10⁷) | Number |
| Ashwagandha. | 115 | 94 | 3.42 × 10¹¹ | 500 | 17.1 | 34.2 | 8 |
| Root | | | | | | | |
| Ashwagandha | 132 | 114 | 1.88 × 10¹¹ | 500 | 9.4 | 18.8 | 7 |
| Stem | | | | | | | |
| Ashwagandha | 128 | 89 | 1.85 × 10¹¹ | 500 | 9.25 | 18.5 | 7 |
| Leaf | | | | | | | |
| Ashwagandha | 149 | 92 | 1.81 × 10¹¹ | 500 | 9.05 | 18.1 | 7 |
| Fruit | | | | | | | |
| Ashwagandha | 142 | 95 | 1.47 × 10¹¹ | 500 | 7.35 | 14.7 | 7 |
| Seed | | | | | | | |
| Ashwagandha | 117 | 83 | 4.22 × 10¹⁰ | 500 | 2.11 | 4.22 | 7 |
| Leaf HS | | | | | | | |
| Ashwagandha | 125 | 99 | 9.78 × 10¹⁰ | 500 | 4.89 | 9.78 | 7 |
| Root HS | | | | | | | |
| Ashwagandha | 108 | 82 | 4.04 × 10¹⁰ | 500 | 2.02 | 4.04 | 7 |
| Stem HS | | | | | | | |

Key: HS = Heat Shock

### Example 2. Extraction of Exosomes from Dried Ashwagandha Seeds

The seeds of the ashwagandha plant were dried and obtained. The seeds are hydrated in DPBS for 3 days at 4 degrees centigrade. The hydrated seeds are frozen at −80° C. for 48 hours. The seeds are thawed and are added to 15-mL Precellys homogenization bead vial containing 1.4 mm and 2.8 mm ceramic beads (CFmix50) followed by the addition of 7 mL sterile PBS followed by homogenization. The samples are pulsed at 8000 rpm for 20 seconds. The samples are removed and placed on dry ice for 45 seconds. This is repeated three times. The samples are pulsed at 10000 rpm for 20 seconds. The samples are removed and placed on dry ice for 45 seconds. The contents are removed to 50-mL conical tubes. The beads are rinsed with 7 mL of PBS which is repeated three times. Volume sample to ˜40 ml. Mix and place at −80° C. until ready for exosome purification. Centrifuge each sample@ 3000 g for 15 minutes. Transfer supernatant to new vials. Centrifuge each sample at 15000 g for 15 minutes. Transfer supernatant to 250-mL bottles. Each sample was filtered through a 0.22-um filter before centrifugation at 100,000×g for 2 hr. Exosomes were characterized using a Thermo NanoDrop spectrophotometer for protein determination and approximate RNA concentration by direct absorbance; exosomes were not lysed, stained, or RNA extracted prior to measurements. Particle diameter and concentration was assessed by Nanoparticle Tracking Analysis (NTA) using a Particle Metrix ZetaView®.

### Example 3. Evaluation of Ashwagandha Seed Derived Exosomes Safety (In Vitro)

Free radical generation of oxygen is a byproduct of cellular respiration and becomes elevated in response to stress and inflammation. Reactive Oxygen Species (ROS) including superoxide, peroxides, and hydroxyl ions can be detected using fluorescent dyes that are selective for different free radicals. Dihydroethidium (DHE) becomes highly fluorescent in the presence of superoxides and peroxides, while CellROX is selective for superoxides. CellROX is a fluorogenic probe for measuring cellular oxidative stress in both live and fixed cell imaging, with absorption/emission maxima at ˜644/665 nm. The cell-permeant dye is non-fluorescent while in a reduced state and exhibits bright fluorescence upon oxidation by reactive oxygen species (ROS). CellROX is very sensitive to O2-radicals formed during cell respiration and show a “relatively” high basal level of detection.

P. acnes exposure induces inflammation and an immediate upregulation in stress response genes that scavenge and reduce the levels of cellular ROS. This response is detected using CellROX by a detectible reduction of ROS after 6 and 24 hours of stress exposure. Modulation of ROS is very transient and can be monitored at earlier time points (1-3 h) to see pharmacological effects. DHE fluorescence accumulates in the cell after exposure to P. acnes. After 24 hours, ROS is elevated compared to media only treatment.

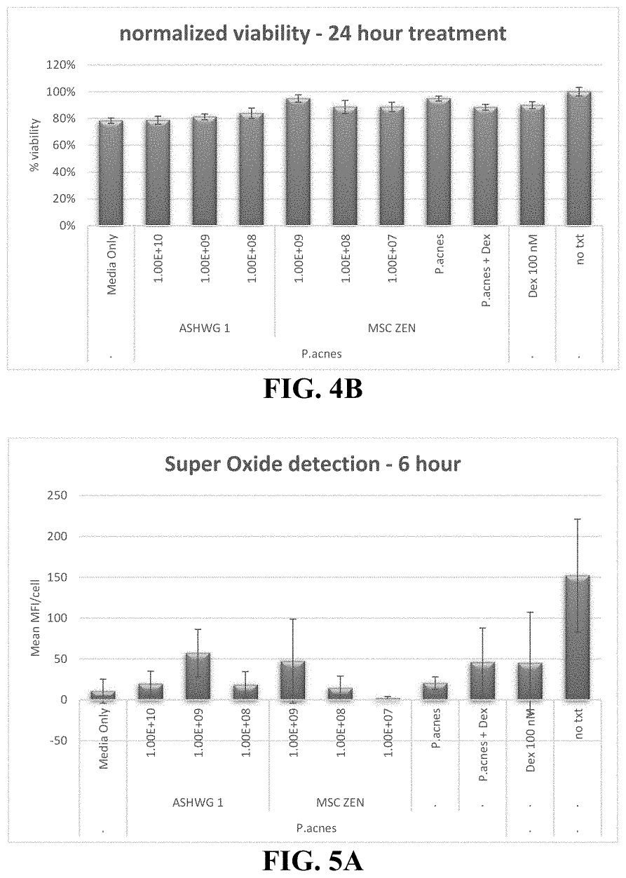

### Viability (See FIG. 4)

Dermal fibroblasts were treated with Ashwagandha Seed Derived Exosomes or Human Adipose Tissue Mesenchymal Stem Cells Derived Exosomes as control, at concentration between 1×10⁸ to 1×10¹⁰, for 6 and 24 hours. After treatment, media was removed and replaced with Cell Titer Blue viability reagent. Viability was measured after 1 hour using a plate reader. Media was removed and cells were washed with warm PBS for imaging assays.

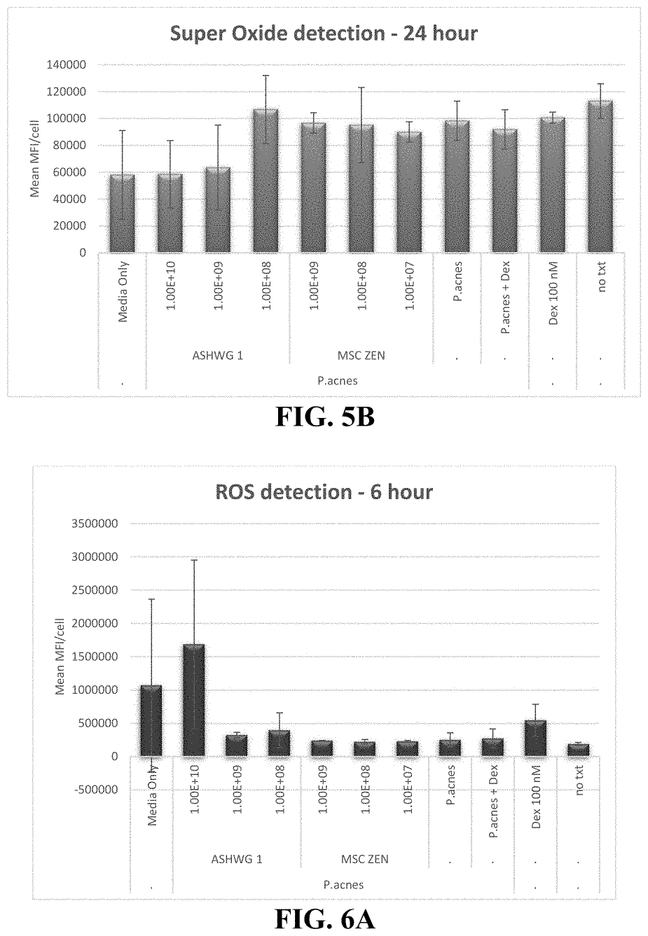

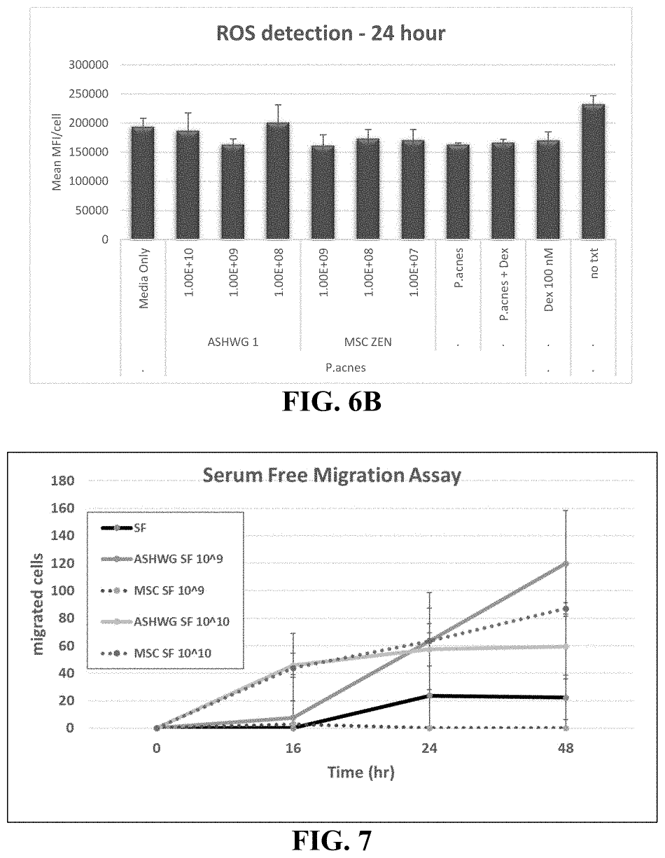

### Oxygen Radical Induced Oxidative Stress (See FIG. 5 and FIG. 6)

After the viability assay, cells were incubated with a non-selective super oxide fluorescent marker Dihydroethidium (DHE), a ROS selective marker, CellROX, and Hoechst nuclear stain. Cells were incubated at 37 C for 30 minutes and imaged using automated High Content Imager. Fluorescent signal per cell was analyzed for each treatment condition and graphed above. Red line denotes P. acnes level for reference across treatments.

### Example 4. Efficacy of Ashwagandha Seed Derived Exosomes (In Vitro)—Cell Migration Test

The Oris™ Cell Migration Assay (Platypus Technologies, Madison, Wis.), is a reproducible, sensitive, and flexible assay that can be used to monitor cell migration. Formatted for a 96-well plate, the assay utilizes Oris™ Cell Seeding Stoppers made from a medical-grade silicone to restrict cell seeding to the outer annular regions of the wells. Removal of the stoppers reveals a 2 mm diameter unseeded region in the center of each well, i.e., the detection zone, into which the seeded cells may then migrate. The Oris™ Detection Mask is applied to the plate bottom and restricts visualization to the detection zones, allowing only cells that have migrated to be detected (see FIG. 1). The Oris™ Cell Migration Assay is designed to be used with any commercially available stain or labeling technique. Readout can be performed by microscopy or use of a microplate reader.

### Experiment 1 (See FIG. 7)

Human Umbilical Vein Endothelial Cells (HuVEC) were seeded onto collagen coated Platypus migration plates and allowed to attach for 2 hours in complete medium (Endothelial Cell Growth Media, Cat #CCM027, R&D Systems). Cells were then washed and fed with serum-free medium alone (negative control, ECGM base medium, without growth factors or serum) or serum free medium supplemented with Ashwagandha Seed Derived Exosomes or Human Adipose Tissue Mesenchymal Stem Cells Derived Exosomes at either 1×10⁹ or 1×10¹⁰ particle/treatment, in triplicate, and incubated at 37° C., 5% CO₂. Cells were stained with Calcein AM prior to imaging at 16, 24, and 48 hours. The number of cells within the defined region were counted for each biological triplicate. Statistical Analysis was performed (2way ANOVA with Bonferroni's multiple comparison test).

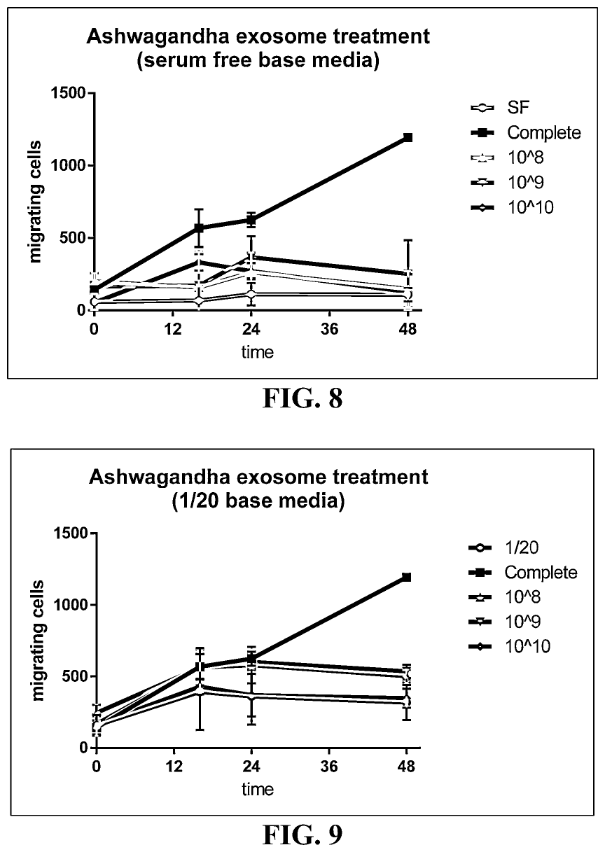

### Experiment 2 (See FIGS. 8 and 9)

Dermal Fibroblast Cells were seeded in triplicates onto collagen coated Platypus migration plates and allowed to attach to surface. Cells were incubated in complete media for 2 hours at 37 C, 5% CO₂ to allow cells to attached. Attached cells were then washed with DPBS to remove all growth factors and serum. Ashwagandha Seed Derived Exosomes at 1×10⁸, 1×10⁹ or 1×10¹⁰ particle/treatment, in triplicate, were prepared either in Serum Free, growth factor free media or in 1/20 complete media (complete media was diluted 1:20 with serum-free, growth factor-free media), and incubated at 37 C, 5% CO₂. Cells were stained with Calcein AM prior to imaging at 0, 16, 24, and 48 hours. The number of cells within the defined region were counted for each biological triplicate. Statistical Analysis was performed (2way ANOVA with Bonferroni's multiple comparison test).

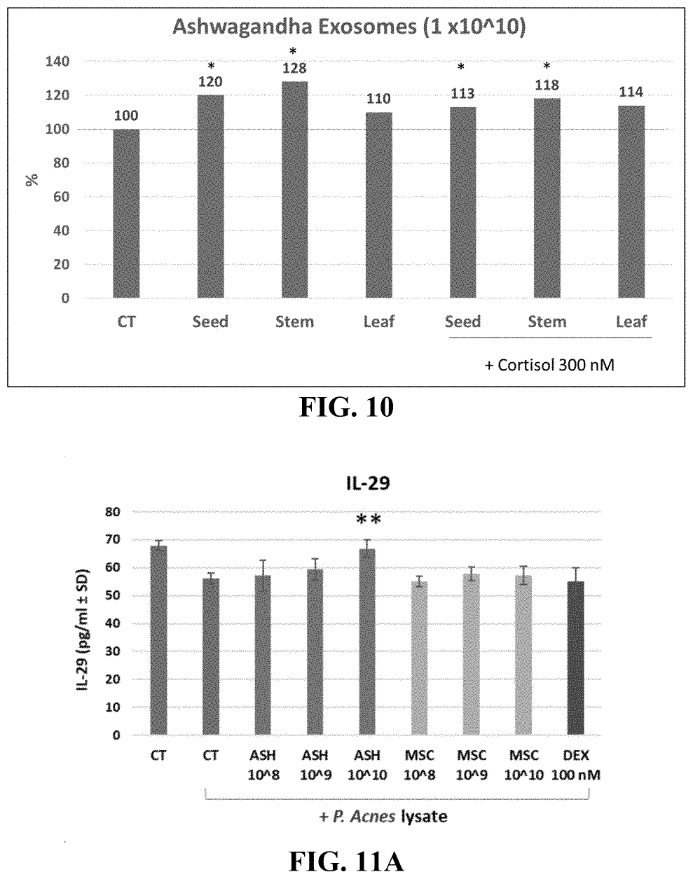

### Experiment 3 (See FIG. 10).

Human Hair Follicle Dermal Papilla Cells (HFDPC) were used at the 6th passage, incubated at 37° C. and 5% CO₂. Cells were cultured in HFDPC basal medium (PromoCell), supplemented with Penicillin (50 U/ml) and Streptomycin (50 μg/ml). In order to determine live cells and count, cell viability was checked by staining with Trypan-Blue Solution. Cell were seeded at 4000 cells/well density in a 96-wells plate. During the experiments, medium was replaced with assay medium (DMEM; L-glutamine, 2 mM; Penicillin, 50 U/ml; Streptomycin, 50 mg/ml; and FCS, 1%).

### Baseline Experiment

HFDPC were seeded in 96-well plate and grown for 24 hours in culture medium. The medium was then replaced by assay medium containing or not (untreated) Ashwagandha Exosomes from Seed, Stem and Leaf at a concentration of 1×10¹⁰. Cells were incubated for 72 hours. BrdU was added for the last 24 hours of incubation. All experimental conditions were performed in n=3.

### Stress Induced Experiment

HFDPC were seeded in 96-well plate and grown for 24 hours in culture medium. The medium was then replaced by assay medium containing or not (untreated) Ashwagandha Exosomes from Seed, Stem and Leaf at a concentration of 1×10¹⁰+cortisol (300 nM). Cells were incubated for 96 hours. BrdU was added for the last 24 hours of incubation. All experimental conditions were performed in n=5.

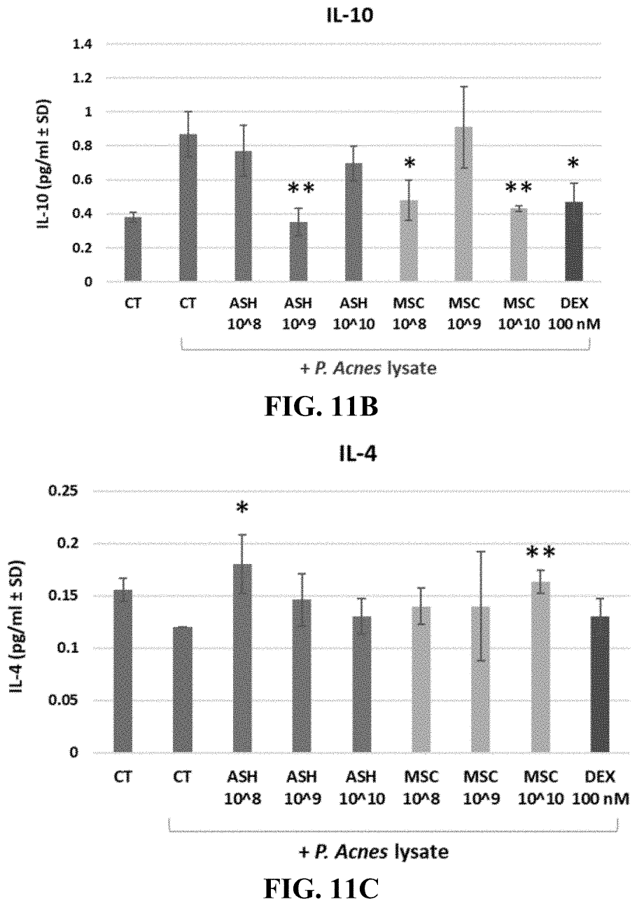

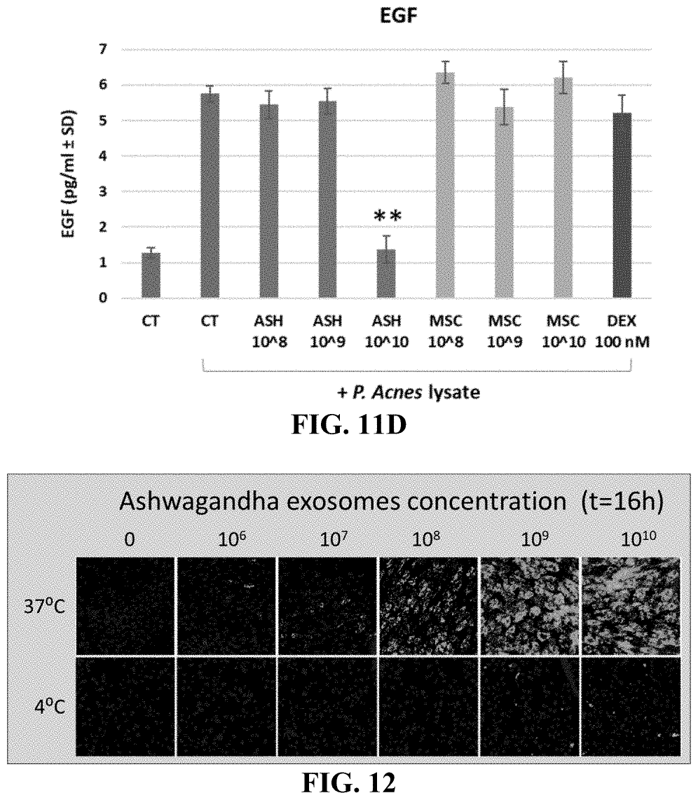

### Anti-Inflammatory Action (See FIG. 11)

Dermal fibroblasts were seeded in 96 well plate at 9000 cells per well. Cells were allowed to attach overnight at 37 C, 5% CO₂. Ashwagandha Seed Derived Exosomes (ASH) were diluted in growth media to a final concentration of 1×10¹⁰, 1×10⁹ and 1×10⁸. As a control, Human Adipo Tissue Mesenchimal Stem Cells Derived Exosomes (MSC) were diluted at the same concentrations. Dexamethasone (DEX) was used as a positive control and diluted to 100 nM in growth medium. ASH, MSC and DEX were incubated for 10 min at 37 C 5% CO₂. Propionibacterium acnes (P. acnes) lysate was used to induce an inflammatory response in cell culture. Cytokine release was induced by activation of Toll-Like Receptor pathways. 2× P. acnes treatment was added in growth media at 90 ug/ml (final 45 ug/ml). After 6 hours, 200 ml of media was collected and stored at −80 C until ELISA assay.

### Results

At 6 hours, P. acnes lysate induced in dermal fibroblasts a significant reduction in IL-29 release compared to vehicle alone. This reduction was prevented with 1×10¹⁰ ASH (statistically significant). Other treatment, including DEX, did not show the same recovery. When tested for IL-10 release, P. acnes lysate induced a 2-fold increase in dermal fibroblasts compared to vehicle alone. This induction was significantly prevented with 1×10⁹ NV/mL of ASH exosomes and 1×10¹⁰ and 1×10⁸ NV/mL of MSC exosomes. DEX showed a significant recovery to baseline as well. When IL-4 release was measured in dermal fibroblasts, P. acnes lysate induced a slight decrease. ASH at 1×10⁸ were able to significantly neutralize the decrease, as well as MSC at 1×10¹⁰. DEX did not show a recovery to baseline. Finally, P. acnes lysate induced a 5-fold increase in EGF protein levels in dermal fibroblasts compared to vehicle alone. This increase was significantly prevented with 1×10¹⁰ ASH exosomes but not with MSC exosomes or DEX.

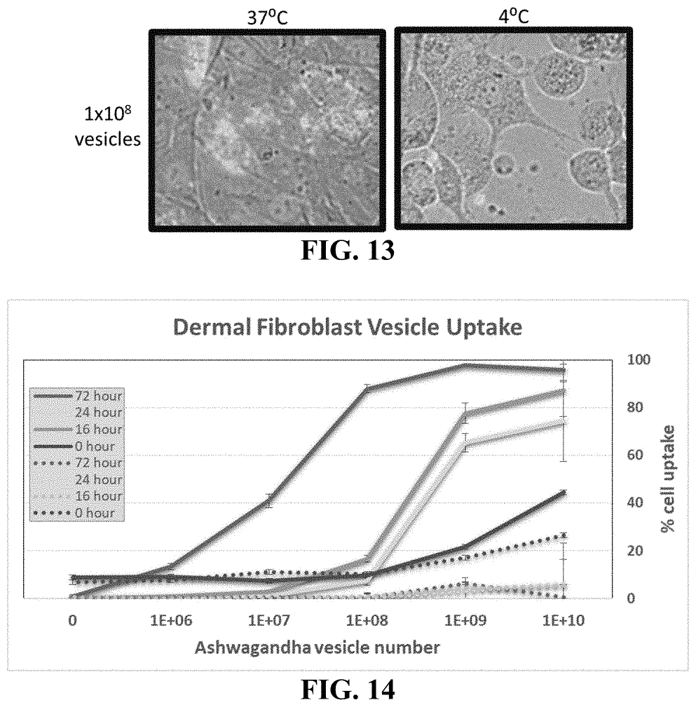

### Example 5. Ashwagandha Seed Derived Exosomes Attachment and Uptake in Dermal Fibroblasts (See FIGS. 12-14)

### Protocol

Ashwagandha exosomes were isolated from the dry seeds. Purified exosomes were labeled with a green, fluorescent lipid dye (Vybrant DiD 650 nm, Thermo cat #V22887). Labeled exosomes were enumerated using ZetaView Particle NanoTracking Analysis.

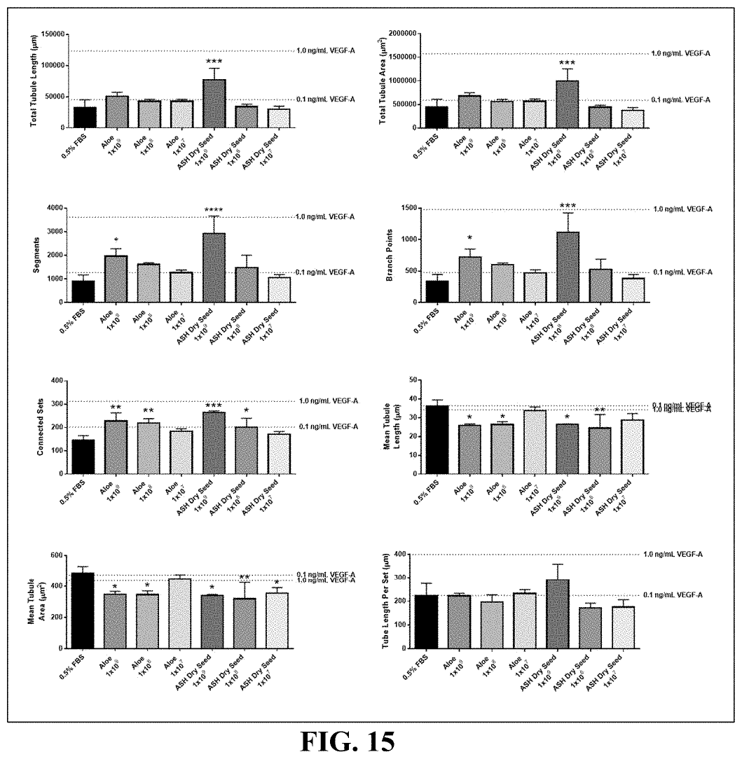

Dermal Fibroblasts were plated in replicate 96 well plates and allowed to attach overnight at 37° C., 5% CO₂. Labeled exosomes were diluted in growth media. Plate 1: Cells were washed with warm PBS, replaced with treatment media and placed at 37° C., 5% CO₂ for 15 minutes to allow nuclear dye to accumulate in the cells. The plate was then imaged at t=0, 16, 24, and 72 hours and the percentage of cells containing the fluorescent dye were tabulated and graphed (see solid line in FIG. 14) Plate 2: Cells were washed with cold PBS, replaced with cold treatment media and placed at 4° C. for 15 minutes to allow nuclear dye to accumulate in the cells. The plate was then imaged at t=0, 16, 24, and 72 hours and the percentage of cells containing the fluorescent dye were tabulated and graphed (see dotted line in FIG. 14).

Labeled Ashwagandha exosomes immediately attached to the dermal fibroblast and began to be taken in (FIG. 12). At 16 hours, a green, fluorescent signal was strongly detected with exosome concentrations 1×10⁸ and nearly 80% of all cells have taken in fluorescent exosomes at 1×10⁹ (FIG. 12). At 72 hours, 1×10⁸ shows ˜90% of cells containing labeled Ashwagandha exosomes.

### Example 6. Tubulogenesis—Human Umbilical Vein Endothelial Cells Tube Formation Using Ashwagandha Seed Derived Exosomes

The purpose of this study was to determine the ability of ASH dry seed exosomes to induce endothelial tube formation in human umbilical vein endothelial cells (HUVEC). One of the most widely used in vitro assays to model the reorganization stage of angiogenesis is the tubulogenesis assay. Angiogenesis is the process by which new blood vessels form, allowing the delivery of oxygen and nutrients to the body's tissues. It is a vital function, required for growth and development as well as the healing of wounds.

### Protocol

Pooled Dermal fibroblasts (Passage 2; Lot DFM012221) were seeded at 10,000 cells per well in a 96-well plate (Perkin Elmer CellCarrier—96 Ultra Microplates). Cells were allowed to grow in normal growth media (DMEM+10% FBS) until confluency, which was approximately 48 hrs. Cells were incubated at 37° C., 5% CO₂. After dermal fibroblasts reached confluency, 2,500 pooled human umbilical vein endothelial cells (HUVECs) (Passage3; ATCC Lot 70032758) were added to each well in 1:1 ratio of dermal fibroblast growth media to endothelial cell growth media. HUVECS were allowed to attach to dermal fibroblast feeding layer for 24 h. After 24 h, the appropriate EVs, VEGF-A titrated media (VEGF-A, Lot), and standard endothelial growth media (ECGM-1) was added to individual wells (n=3). All samples except the titrated VEGF-A media contained 0.5% FBS. VEGF-A media contained 0.1% BSA. Media was changed every 2 days for 7 days. Cells were incubated at 37° C., 5% CO₂. After 7 days in incubation, media was removed, and cells were washed 2× with DPBS. Next, cells were fixed with 4% paraformaldehyde for 30 min at room temperature. Following fixation, cells were washed 2× with DPBS. Next, cells were permeabilized with 0.3% Triton X-100 for 5 min at room temperature. After permeabilization, cells were blocked with normal human serum for 1 h at room temperature. Following blocking with serum, cells were washed 1× with DPBS and stained with 1:200 CD31-AlexaFluor 647 (Abcam ab215912) in DPBS for 2 h at room temperature. After staining, cells were washed 2× with DPBS, and 100 μL of 50/50 Glycerol/PBS was added to each cell to preserve cell staining. HUVEC tubulogenesis was imaged using the ImageXpress Pico Automated Cell Imaging System (Molecular Devices). Tubulogenesis was analyzed using the preprogrammed angiogenesis network analysis template (Molecular Devices).

### Results

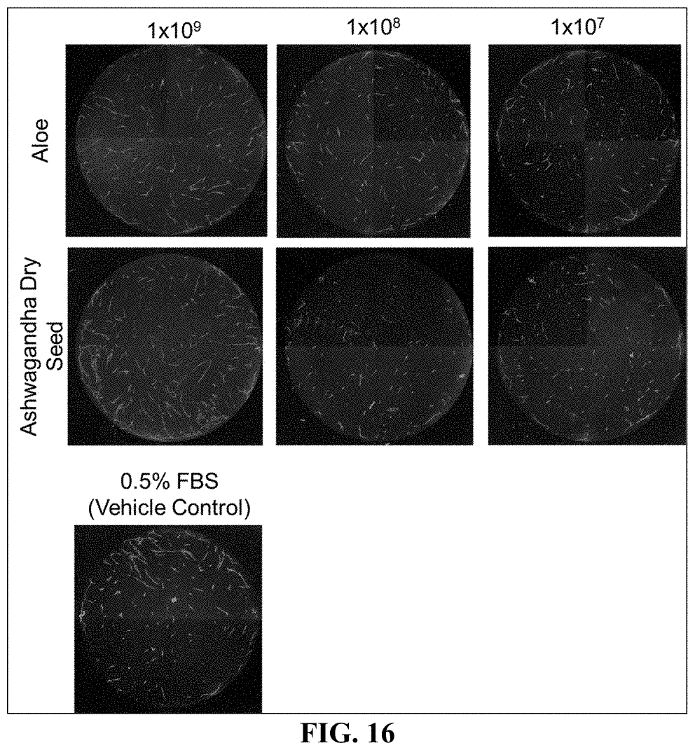

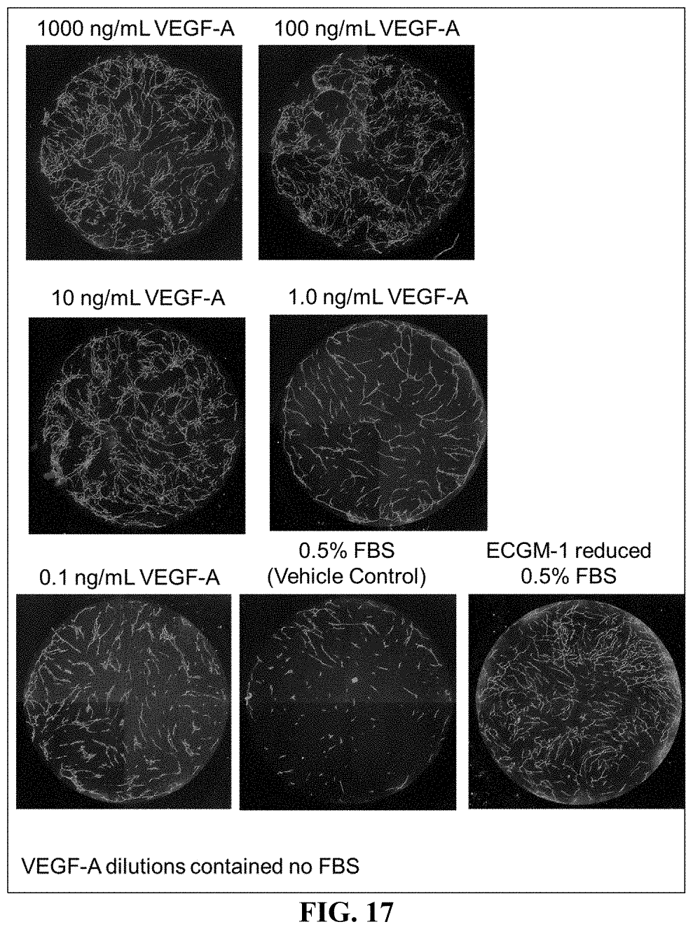

ASH dry seed derived exosomes promoted tubulogenesis significantly (see FIGS. 15 and 16). Their effect was comparable to VEGF (a known promoter of tubulogenesis and angiogenesis) that was used as a positive control (FIGS. 15 and 17). ASH dry seed derived exosomes efficacy was superior to Aloe leaf derived exosomes for most parameters analyzed (see FIGS. 15 and 16).

### Example 7. Electron Microscopy Preparation for Ashwagandha Seed Derived Exosomes or ELN (Exosome Like Nanovescicles)

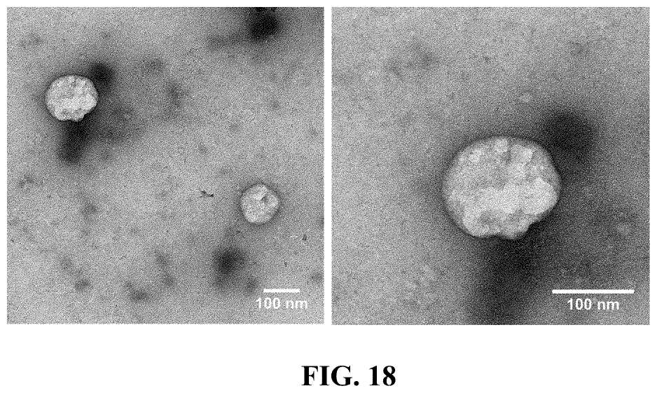

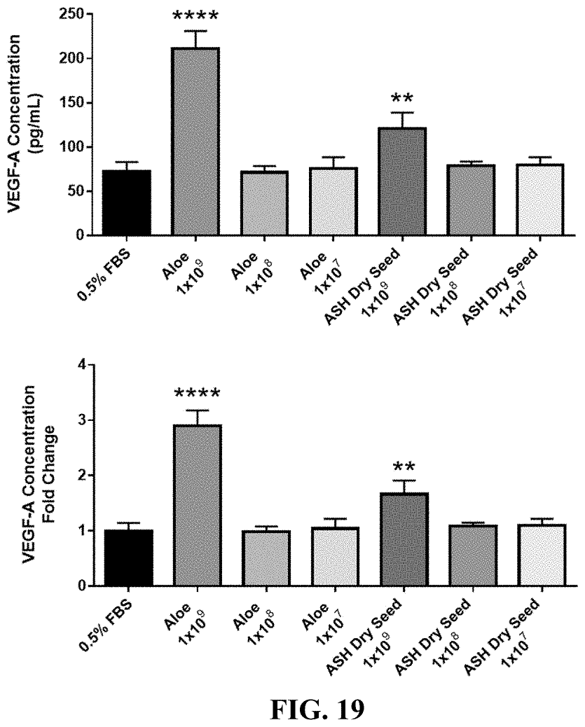

Ashwagandha Seed Derived Exosomes or ELN sample (5 μL; diluted 1:1 with 0.05M PBS) was applied to the carbon side of the 300-mesh copper grid, which was previously glow discharged for 30 seconds. The sample adsorbed to the grid surface for 30 seconds to 1 minute. The grid was wicked with a piece of filter paper and allow capillary action to pull off excess sample. Two drops of filtered 1% aqueous uranyl acetate were placed a sheet of parafilm. Each grid was touched to one drop of UA and immediately wicked with filter paper and repeated a second time. Grids air dried completely before viewing in the TEM. The resulting image can be seen in FIG. 18.

### Example 8. VEGF-A Induction by Aloe Leaf Derived Exosome/ELN and Ashwagandha Seed Derived Exosome/ELN

Pooled human dermal fibroblasts were cultured until 70% confluent in standard growth media (DMEM-HG+10% FBS). After cells reached 70% confluency, cells were washed 1× with PBS and serum starved overnight (18 h) in 0.5% FBS. After serum starvation, cells were washed 1× with PBS and treated for 48 h with 1×10⁹, 1×10⁸, 1×10⁷ particles per 100 μL of Aloe ELN or Ashwagandha ELN. All ELN conditions were incubated in 0.5% FBS+DMEM-HG. Cells were also incubated. All treatments were performed in biological triplicates. After treatment, the conditioned media was measured for VEGF-A using a Human VEGF Quantikine ELISA Kit (R&D Systems, DVE00) following the manufacturer's protocols. The basal concentration of VEGF-A within the EV dosage media, 0.5% FBS vehicle control, and 10% FBS positive control was measured for background subtraction. Only VEGF-A was detected in the 10% FBS and was subtracted from the conditioned media VEGF-A concentration.

### Example 9. Melanogenesis Stimulation by Ashwagandha Seed Derived Exosomes/ELN

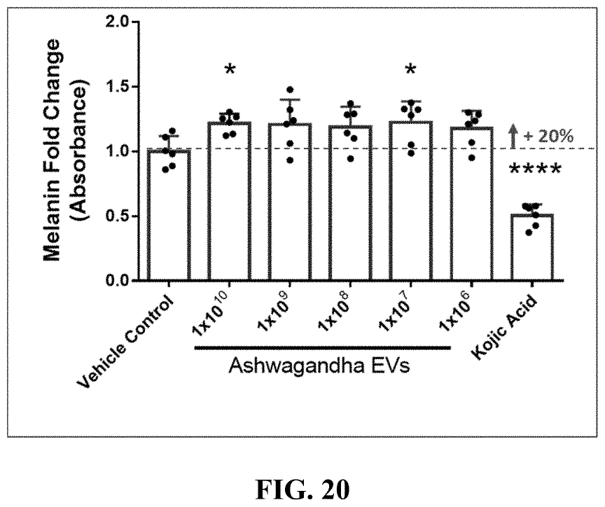

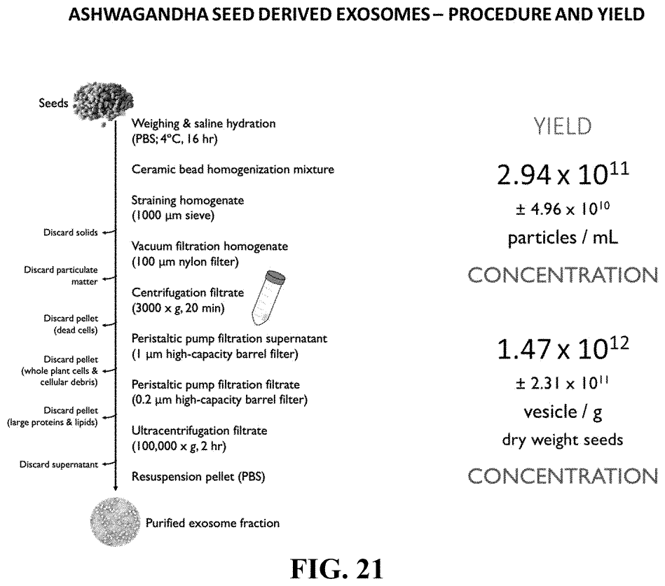

B16 mouse melanoma cells were plated and grown in the 96-well tissue culture plates. Controls included Kojic Acid (positive, B16 cells), and vehicle alone (negative). Cells were treated with 1×10⁶, 1×10⁷, 1×10⁸, 1×10⁹, or 1×10¹⁰ Ashwagandha dry seed derived exosomes for 3 days. Following the treatment period, the level of pigment produced was quantified using a microplate reader at 540 nm. To monitor cell viability MTT conversion method was used, which measures the reduction of MTT dye from yellow, water-soluble, tetrazolium salt to a bluish-purple insoluble formazan precipitate. The intensity of blue color is indicative of cell viability. After quantifying the amount of pigment produced, the medium was removed, and the cells exposed to MTT solution for two hours. Formazan material was solubilized with reagent alcohol (95% ethanol: 5% isopropanol) and shaken on an orbital shaker for 30 minutes. Dye uptake and conversion by viable cells was determined by measuring the extracted formazan at 570 nm. Total pigmentation was determined by normalizing the levels of pigment with the levels of cell viability. Results can be seen in FIG. 20.

### Example 10. Penetration of Fluorescently Labelled Ashwagandha Seed Derived Exosomes in Micro-Dissected Hair Follicle During Organ Culture

Twenty-four to 30 micro-dissected Skin Explants containing Human Hair Follicles are run for 4 experimental groups (vehicle, 3 concentrations of labelled-exosome formulation). Two time points during organ culture are evaluated and in vitro output that is measured is imaging for the labelled exosomes.

### Example 11. Effect of Ashwagandha Seed Derived Exosome on Micro-Dissected Hair Follicle (HFs) During Organ Culture. Hair Growth and Pigmentation

Twenty-five to 30 micro-dissected HFs are removed from one donor. At one time point during organ culture, hair follicle elongation (hair shaft production) is measured ex vivo (digital brightfield microscope). Furthermore, the following parameters are evaluated in situ:
1. Microscopic hair cycle (Ki-67/TUNEL or caspase 3, Masson Fontana), also for selecting anagen HFs
2. Hair matrix proliferation and apoptosis (Ki-67/TUNEL immunofluorescence)
3. Hair cycle-independent hair follicle melanin content (Masson Fontana histochemistry)
4. Hair cycle-independent melanosome formation (Gp100 immunofluorescence)
5. Hair cycle-independent tyrosinase activity (tyrosinase in situ zymography)

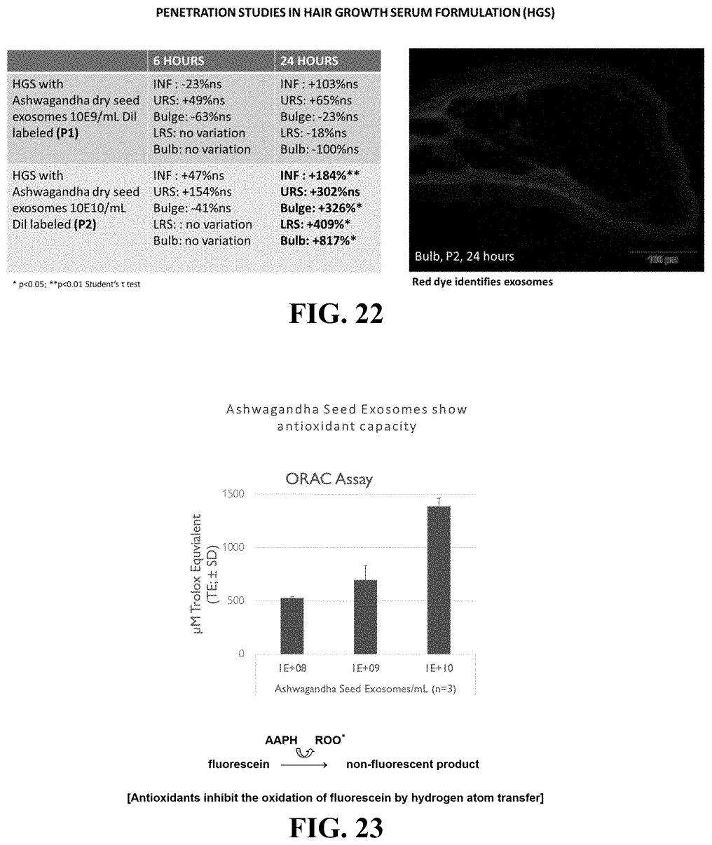

### Example 12. Hair Follicle Penetration (See FIG. 22)

The penetration of two formulations (Hair Growth Serum/P1 and Hair Growth Serum/P2) containing Ashwagandha dry seed exosomes labeled with a dye (Dil) at 2 concentrations (1×10⁹/mL=P1 and 1×10¹⁰/mL=P2) and 2 timepoints throughout human hair follicles.

Hairy human abdominal skin with adipose tissue was used. The ex vivo phase allows reproduction of topical application of the test products. The histological phase allows to evaluate modulation of biological parameters by staining and immunostaining. The penetration of the product was assessed by microscopic analysis of product's fluorescence in hair follicle.

### Procedure

The study was conducted on a human hairy skin explants with adipose tissue obtained from an abdominoplasty of a 27-year-old Caucasian male (P2406-AB27) with a phototype II (according to Fitzpatrick skin color classification) and frozen two months at −20° C. before the study beginning. 12 explants of 20 mm-diameter were prepared and kept in survival in BEM culture medium (BIO-EC's Explants Medium) at 37° C. in a humid, 5%-CO₂ atmosphere.

After 1 h of skin thawing, the tested products containing Ashwagandha dry seed exosomes 1×10⁹/mL Dil labeled (P1) and containing Ashwagandha dry seed exosomes 1×10¹⁰/mL Dil labeled (P2) were applied topically on the basis of 6 μl per explant (2 μl/cm²) and spread using a small spatula. The control batch (T) did not receive any treatment.

On day 0+6 h (6 h after product application) and day 1 (24 h after product application), the explants of all batches were collected and frozen at −80° C., in the dark.

The frozen samples were cut into 7-μm-thick sections using a Leica CM 3050 cryostat. Serial sections were then mounted on histological glass slides.

The microscopical observations were realized using an Olympus BX43 microscope. Pictures were digitized with a numeric DP72 Olympus camera with cellSens storing software. The penetration of the tested DIL-labeled product was analyzed along the hair follicles on frozen hairy skin sections, using a DIL-specific microscope filter (DIL: Absorbance wavelength: 549 nm, Emission wavelength 565 nm).

### Results

Using a DIL-specific filter, a red fluorescence was observed in all areas of the hair follicle: infundibulum (INF), upper root sheaths (URS), bulge, lower root sheaths (LRS) and bulb, for all conditions.

Based on the observations of the blank batch at 6 h (T-6 h) and 24 h (T-24 h), a red autofluorescence was observed particularly in the hair shaft and in the sebaceous glands, located between the upper root sheaths (URS) and the bulge areas. So, the red signal that has been observed on the blank batch at 6 H and 24 H is to be considered as no specific signal (background). If DIL-specific staining exhibits a low fluorescence intensity, close or lower to the one observed on the blank batch T, it will not be possible to discriminate this specific red signal from the autofluorescence (background). That is why, a specific DIL detection is considered only if the red fluorescence is significantly higher than the one observed on the blank batch T at 6 h or 24 h. The product containing Ashwagandha dry seed exosomes 1×10⁹/mL Dil labeled (P1) does not increase significantly the DIL-specific fluorescence in any part of the hair follicles, suggesting that the product P1 cannot be specifically detected.

The product containing Ashwagandha dry seed exosomes 1×10¹⁰/mL Dil labeled (P2) increases significantly the DIL-specific fluorescence in all parts of the hair follicles after 24 h. These results indicate that the product containing Ashwagandha dry seed exosomes 1×10¹⁰/mL Dil labeled (P2) can be specifically detected and can penetrate throughout the hair follicle after 24 hours.

### Example 13. ORAC (Oxygen Radical Absorbance Capacity) Antioxidant Assay (See FIG. 23)

The ORAC Antioxidant Assay measures the loss of fluorescein fluorescence over time due to peroxyl-radical formation by the breakdown of AAPH (2,2′-azobis-2-methyl-propanimidamide, dihydrochloride). Trolox [6-Hydroxy-2,5,7,8-tetramethylchroman-2-carboxylic acid], a water soluble vitamin E analog, serves as a positive control inhibiting fluorescein decay in a dose dependent manner. The ORAC assay is a kinetic assay measuring fluorescein decay and antioxidant protection over time. The antioxidant activity in biological fluids, cells, tissues, and natural extracts can be normalized to equivalent Trolox units to quantify the composite antioxidant activity present.

Principle of Assay A peroxyl radical (ROO⁻) is formed from the breakdown of AAPH (2,2′-azobis-2-methyl-propanimidamide, dihydrochloride) at 37° C. The peroxyl radical can oxidize fluorescein (3′,6′-dihydroxy-spiro[isobenzofuran-1[3H], 9′[9H]-xanthen]-3-one) to generate a product without fluorescence. Antioxidants suppress this reaction by a hydrogen atom transfer mechanism, inhibiting the oxidative degradation of the fluorescein signal. The fluorescence signal is measured over 30 minutes by excitation at 485 nm, emission at 538 nm, and cutoff=530 nm. The concentration of antioxidant in the test sample is proportional to the fluorescence intensity through the course of the assay and is assessed by comparing the net area under the curve to that of a known antioxidant, Trolox.

### Procedure

1. Fluorescein working solution was prepared from stock solution and protected from the light.
2. Trolox standards were prepared for final concentrations of 100, 50, 25, 12.5, and 6.25 μM. PBS was designated as blank standard.
3. 150 μl of the working fluorescein solution was added to each of the inner 60 wells of the black clear bottom assay plate.
4. 25 μl of samples or Trolox standards was added to each individual assay well according to the ORAC template plate map. 25 μl of PBS was added to individual wells as a negative control (Plate Blank). Plate was placed at 37° C. for at least 10 minutes in the plate reader chamber or incubator.
5. While the assay plate equilibrated to 37° C., AAPH working solution was prepared and place on ice until needed.
6. The assay was initiated by adding 25 μl of the AAPH working solution to each of the wells containing standards and samples.
7. A kinetic read was performed on the plate for 30 minutes with 1-minute intervals at excitation=485 nm and emission=528 nm.
8. Upon completion of the assay area under the curve (AUC), net AUC and μM Trolox values for were calculated each unknown.

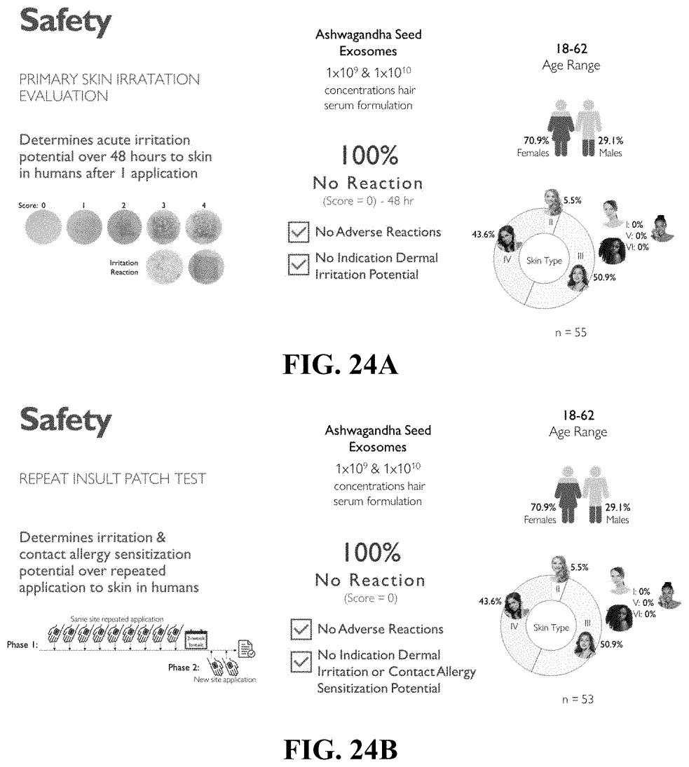

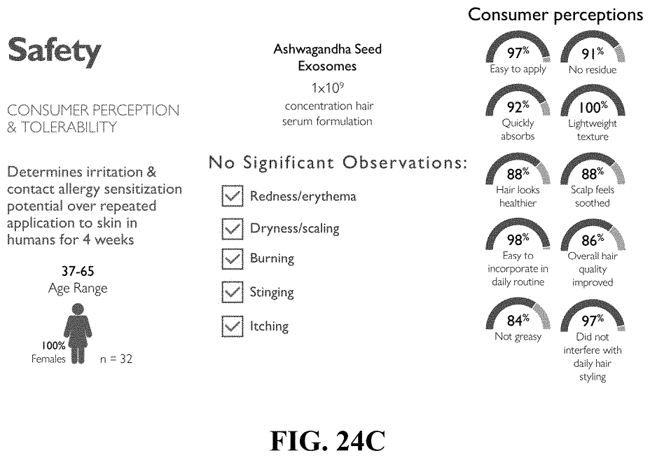

### Example 14. Primary Skin Irritation Evaluation (See FIG. 24A)

### Objective

To determine the primary (acute) irritation potential of a test material (hair growth serum comprising ashwagandha exosomes/ELNs 1×10⁹ or 1×10¹⁰ after a single application to the skin of human subjects for 48 hours.

### Inclusion in a Study

Individuals who were not currently under a doctor's care.
Individuals who were free of any dermatological or systemic disorder that would interfere with the results, at the discretion of the Investigator.
Individuals who were free of any acute or chronic disease that would interfere with or increase the risk on study participation.
Individuals who completed a preliminary medical history form and were in general good health.
Individuals who read, understood and signed an informed consent document relating to the specific type of study.
Individuals who were able to cooperate with the Investigator and research staff, and were willing to have test materials applied according to the protocol, and complete the full course of the study.
   Exclusion from a Study
Individuals who were under 18 years of age.
Individuals who were currently under a doctor's care.
Individuals who were currently taking any medication (topical or systemic) that might mask or interfere with the test results.
Individuals who had a history of any acute or chronic disease that might interfere with or increase the risk associated with study participation.
Individuals who were diagnosed with chronic skin allergies.
Female volunteers who indicated that they were pregnant or nursing.

| Number of subjects enrolled | 55 |
|---|---|
| Number of subjects completing study | 55 |
| Age Range | 18-62 |
| Male | 16 |
| Female | 39 |
| Fitzpatrick Skin Type* | |
| 1-always burn, does not tan | 0 |
| 2-burn easily, tan slightly | 3 |
| 3-burn moderately, tan progressively | 28 |
| 4-burn a little, always tan | 24 |
| 5-rarely burn, tan intensely | 0 |
| 6-never burn, tan very intensely | 0 |

### Equipment:

Test materials to be tested under occlusive conditions were placed on an 8-millimeter aluminum Finn Chamber (Epitest Ltd. Oy, Tuusula, Finland) supported on Scanpor Tape (Norgesplaster A/S, Kristiansand, Norway) or an 8-millimeter filter paper coated aluminum Finn Chamber AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRA closed cell system which is made of additive-free polyethylene plastic foam with a filter paper incorporated (It is supplied in units of 10 chambers on a hypoallergenic non-woven adhesive tape; the width of the tape is 52 mm and the length is 118 mm) or other equivalents.

Test materials to be tested under semi-occlusive conditions were placed on a test strip with a Rayon/Polypropylene pad or on a 7.5 mm filter paper disc affixed to a strip of hypoallergenic tape (Johnson & Johnson 1 inch First Aid Cloth Tape).

Test materials to be tested in an open-patch were rubbed directly onto skin for approximately one (1) minute. Approximately 0.02-0.05 mL (in case of liquids) and/or 0.02-0.05 gm (in case of solids) of the test material was used for the study. Liquid test material was dispensed on a 7.5 mm paper disk, which fit in the Finn Chamber.

### Procedure

Subjects were requested to bathe or wash as usual before arrival at the facility. Patches containing the test material were then affixed directly to the skin of the intrascapular regions of the back, to the right or left of the midline and subjects were dismissed with instructions not to wet or expose the test area to direct sunlight. Patches remain in place for 48 hours. Subjects were instructed not to remove the patches prior to their next scheduled visit. Trained skin grading laboratory personnel removed the patch and evaluated the test sites. In the event of an adverse reaction, the area of erythema and edema is measured. The edema is estimated by the evaluation of the skin with respect to the contour of the unaffected normal skin.

### Scoring

Scoring scale and definition of symbols shown below are based on the scoring scheme according to the International Contact Dermatitis Research Group scoring scale. Clinical evaluations are performed by an investigator or designee trained in the clinical evaluation of the skin. Whenever feasible, the same individual does the scoring of all the subjects throughout the study and is blinded to the treatment assignments and any previous scores.
0 no reaction (negative)
1 erythema throughout at least ¾ of patch area
2 erythema and induration throughout at least ¾ of patch area
3 erythema, induration and vesicles
4 erythema, induration and bullae
D Site discontinued
Dc Subject discontinued
DcI Subject discontinued per Investigator

### Observation

No adverse reactions of any kind were reported during the course of this study—for both the 1×10⁹ and 1×10¹⁰ ashwagandha-derived exosome/ELN hair growth formulations. There were six (6) subjects with a Grade 1 reaction to the positive control (2.0% Sodium Lauryl Sulfate Solution). No subjects showed any signs of reaction to the negative control (DI Water).

### Example 15. Repeat Insult Patch Test Evaluation (See FIG. 24B)

### Objective

To determine the irritation and sensitization (contact allergy) potential of a test material (high growth serum comprising ashwagandha exosomes/ELNs 1×10⁹ or 1×10¹⁰ after repeated application to the skin of human subjects.

### Inclusion in a Study

Individuals who were not currently under a doctor's care.
Individuals who were free of any dermatological or systemic disorder that would interfere with the results, at the discretion of the Investigator.
Individuals who were free of any acute or chronic disease that would interfere with or increase the risk on study participation.
Individuals who completed a preliminary medical history form and were in general good health.
Individuals who read, understood and signed an informed consent document relating to the specific type of study.
Individuals who were able to cooperate with the Investigator and research staff, and were willing to have test materials applied according to the protocol, and complete the full course of the study.
   Exclusion from a Study
Individuals who were under 18 years of age.
Individuals who were currently under a doctor's care.
Individuals who were currently taking any medication (topical or systemic) that might mask or interfere with the test results.
Individuals who had a history of any acute or chronic disease that might interfere with or increase the risk associated with study participation.
Individuals who were diagnosed with chronic skin allergies.
Female volunteers who indicated that they were pregnant or nursing.

| | |
|---|---|
| Number of subjects enrolled | 55 |
| Number of subjects completing study | 53 |
| Age Range | 18-62 |
| Male | 15 |
| Female | 38 |
| Fitzpatrick Skin Type* | |
| 1-always burn, does not tan | 0 |
| 2-burn easily, tan slightly | 3 |
| 3-burn moderately, tan progressively | 26 |
| 4-burn a little, always tan | 24 |
| 5-rarely burn, tan intensely | 0 |
| 6-never burn, tan very intensely | 0 |

### Equipment:

Test materials to be tested under occlusive conditions were placed on an 8-millimeter aluminum Finn Chamber (Epitest Ltd. Oy, Tuusula, Finland) supported on Scanpor Tape (Norgesplaster A/S, Kristiansand, Norway) or an 8-millimeter filter paper coated aluminum Finn Chamber AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRA closed cell system which is made of additive-free polyethylene plastic foam with a filter paper incorporated (It is supplied in units of 10 chambers on a hypoallergenic non-woven adhesive tape; the width of the tape is 52 mm and the length is 118 mm) or other equivalents.

Test materials to be tested under semi-occlusive conditions were placed on a test strip with a Rayon/Polypropylene pad or on a 7.5 mm filter paper disc affixed to a strip of hypoallergenic tape (Johnson & Johnson 1 inch First Aid Cloth Tape).

Test materials to be tested in an open-patch were rubbed directly onto skin for approximately one (1) minute. Approximately 0.02-0.05 mL (in case of liquids) and/or 0.02-0.05 gm (in case of solids) of the test material was used for the study. Liquid test material was dispensed on a 7.5 mm paper disk, which fit in the Finn Chamber.

### Procedure

Subjects were requested to bathe or wash as usual before arrival at the facility. Patches containing the test material were then affixed directly to the skin of the intrascapular regions of the back, to the right or left of the midline and subjects were dismissed with instructions not to wet or expose the test area to direct sunlight. Patches remained in place for 48 hours after the first application. Subjects were instructed not to remove the patches prior to their 48-hour scheduled visit. Thereafter, subjects were instructed to remove patches 24 hours after application for the remainder of the study.

This procedure was repeated until a series of nine (9) consecutive, 24-hour exposures had been made three (3) times a week for three (3) consecutive weeks. Prior to each reapplication, the test sites evaluated by trained laboratory personnel. Following a 10-14 day rest period a retest/challenge dose was applied once to a previously unexposed test site. Test sites were evaluated by trained laboratory personnel 48 and 96 hours after application. In the event of an adverse reaction, the area of erythema and edema were measured. Edema is estimated by the evaluation of the skin with respect to the contour of the unaffected normal skin. Subjects were instructed to report any delayed reactions that might occur after the final reading.

### Scoring

Scoring scale and definition of symbols shown below are based on the scoring scheme according to the International Contact Dermatitis Research Group scoring scale. Clinical evaluations are performed by an investigator or designee trained in the clinical evaluation of the skin. Whenever feasible, the same individual does the scoring of all the subjects throughout the study and is blinded to the treatment assignments and any previous scores.
0 no reaction (negative)
1 erythema throughout at least ¾ of patch area
2 erythema and induration throughout at least ¾ of patch area
3 erythema, induration and vesicles
4 erythema, induration and bullae
D Site discontinued
Dc Subject discontinued
DcI Subject discontinued per Investigator

### Observation

No adverse reactions of any kind were reported during the course of this study—for both the 1×10⁹ and 1×10¹⁰ ashwagandha-derived exosome/ELN hair growth formulations. There were six (6) subjects with a Grade 1 reaction to the positive control (2.0% Sodium Lauryl Sulfate Solution). No subjects showed any signs of reaction to the negative control (DI Water).

### Example 16. Consumer Perception and Tolerability Evaluation (See FIG. 24C)

### Objective

Assessment of the cutaneous acceptability (i.e. tolerance) by evaluator grading of erythema/redness and dryness/scaling (individual assessments) of the test product following 4 weeks of consecutive test product use by both men and women.

Assessment of the cutaneous acceptability (i.e. tolerance) by subject rating their level of discomfort for burning, stinging, and itching (individual responses for each parameter) following each use of the test product during 4 weeks of consecutive test product use by both men and women.

Assessment of consumer perception by subject following 4 weeks of consecutive test product use by both men and women.

### Test Product Use Instructions

Apply one dropper full to the entire scalp once per day to clean, dry or damp hair. Position the dropper directly onto the scalp. To distribute the serum evenly, lightly squeeze in a straight line from the front of the hairline to the back of the neck working through sections. There should be a total of about 6 sections. Massage serum into the scalp for 10-15 s. Do not rinse. Can style as per usual. One bottle lasts approximately 30-45 days.

### Assessment

### Clinical Grading of Tolerance

### By Evaluator

Subjects' overall scalp is visually evaluated at the baseline and week 4 interval for erythema/redness and dryness/scaling using the following scoring scale:

Score Severity Erythema/Redness Dryness/Scaling
0 None Normal color No sign
1 Mild Light pink appearance Slight scaling, slight appearance of roughness
2 Moderate Red appearance Small to large scales uniformly distributed with moderate appearance of roughness
3 Severe Deep red to purple appearance Large scales uniformly distributed with severe appearance of roughness. If noted, any other signs of visible irritation are recorded.

### By Subject

Subjects are asked to rate their level of discomfort for burning, stinging, and itchin (individual responses for each parameter) at the baseline and week 4 interval using the following scoring scale:

Score Level of Discomfort
0 No recognizable discomfort
1 Mild discomfort
2 Moderate discomfort
3 Severe discomfort

If noted, any other signs of skin discomfort are recorded.

### Procedure

1. Subjects report to the facility at the start of the study.

2. Prior to beginning all study related activities, prospective subjects completed an informed consent form, medical history form, code of conduct form, and a HIPAA form.

3. Subjects are enrolled on the basis of the inclusion and exclusion criteria. Subjects failing to meet criteria are dismissed from the study.

4. Enrolled subjects are given specific instructions to continue use of their current hair/scalp care regimen and to not introduce any new hair/scalp care products into their normal routine for the duration of the study (with the exception of the test product).

5. Subjects are instructed to replace their current hair/scalp product with the similar test product assigned, if applicable, for the duration of the study.

6. If enrolled, subjects have the below evaluations:

Baseline (Pre-Treatment)

a. Tolerance assessment by evaluator and subject—subjects must meet inclusion/exclusion criteria to continue on the study. Subjects that do not meet criteria are dismissed

7. Following baseline measurements, subjects are given the test product with product use and application instructions. Subjects are also provided with a Subject Evaluation Form to complete following each use of the test product.

8. Subjects are dismissed from the testing facility and informed to return 4 week (±3 days) post-treatment.

9. Subjects are questioned if there have been any changes to their medical information since their last visit. If noted, adverse events are be collected and evaluated.

10. Test products are be collected and weighed, and each participant be interviewed to confirm compliance with the study protocol and provided instructions. Subject Evaluation Forms are collected and reviewed for completeness. Subjects suspected of noncompliance are dismissed from study participation.

11. Subjects have the below evaluations performed:

Week 4 (±3 days) post-treatment
a. Tolerance assessment by evaluator and subject
b. Self-assessment questionnaire

12. Subjects are dismissed from the study following completion of the week 4 testing interval.

### Results Summary

Under conditions of the study a total of 32 healthy female subjects, 37-65 years of age, completed the clinical study evaluating the tolerance of the 1×10⁹ and 1×10¹⁰ ashwagandha-derived exosome/ELN hair growth formulations.

### Clinical Findings:

There were no statistically significant observations of erythema/redness or dryness/scaling from baseline at the week 4 post-treatment interval.

There were no statistically significant reports of burning, stinging, or itching from baseline at the week 4 post-treatment interval.

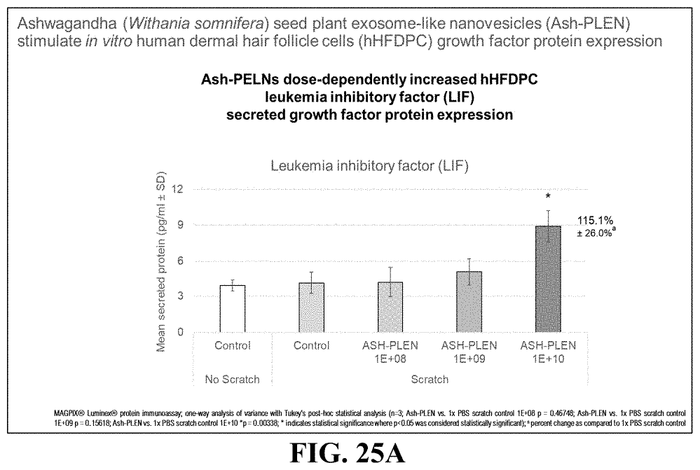

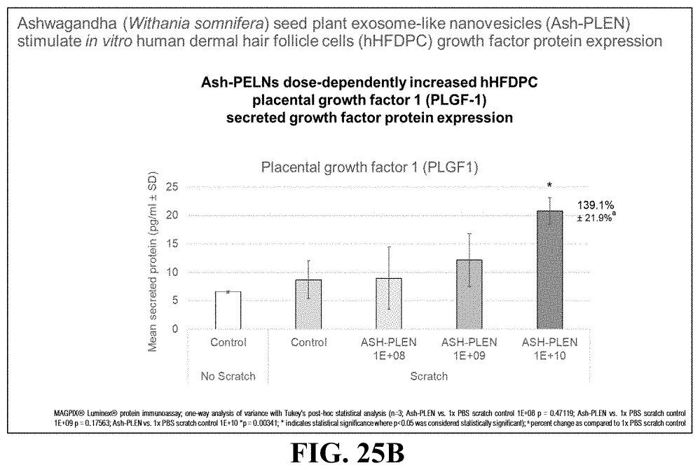

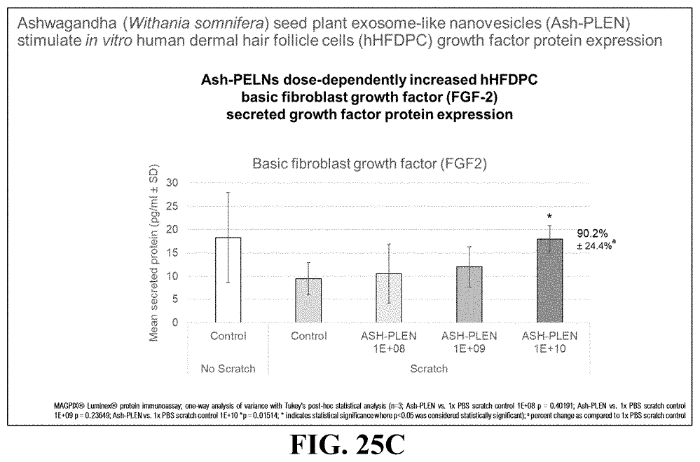

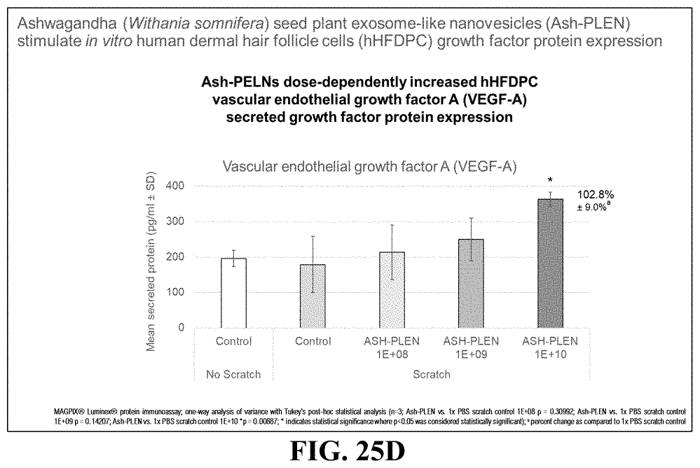

### Example 17. Evaluation of Secreted Growth Factors in Wounded Human Follicle Dermal Papilla Cells (Scratch Assay) Following Treatment with Ashwagandha Nanovesicles (See FIGS. 25A-D)

### Methods

Human Follicle Dermal Papilla cells (HFDPC) were seeded at 10,000 cells/cm² and allowed to attach overnight in growth medium (611K-500). Cells were cultured until reaching 90% confluence at which point the cells were scratched with a p200 tip to create a zone with no cells. The cells were then washed once with PBS to remove cell debris. Ashwagandha seed nanovesicles were diluted in serum and supplement free culture medium to a concentration of 1×10⁸, 1×10⁹, and 1×10¹⁰ particles/mL and added to the wells in triplicate. Unscratched and scratched wells were treated with vehicle (PBS) and served as the assay basal controls. 2% FCS was used as positive control. After 30 hours of treatment, condition medium was removed and centrifuged at 14,000 rpm for 10 mins at 4 C to remove cell debris. The medium was stored then at −80 C until the assay was performed. The attached cells were lysed in 50 ul of NP40 lysis buffer and stored at −80 C for later experiments. Samples to be analyzed using the Growth Factor 11-Plex Human ProcartaPlex™ Panel (Thermofisher Scientific) included the following targets: BDNF, EGF, FGF-2, HGF, LIF, NGF beta, PDGF-BB, PLGF-1, SCF, VEGF-A, VEGF-D. The plate was analyzed on a Luminex MAGPIX System. One-way analysis of variance with Tukey's post-hoc statistical analysis was performed (n=3; p<0.05 considered significant).

### Results

Ashwagandha nanovesicles (Ash-PELNs) dose-dependently increased HFDPC growth factor expression, specifically increasing secreted leukemia inhibitory factor (LIF) (shown in FIG. 25A), placental growth factor 1 (PLGF-1) (shown in FIG. 25B), basic fibroblast growth factor (FGF-2) (shown in FIG. 25C) and vascular endothelial growth factor A (VEGF-A) (shown in FIG. 25D), with significant data at 1×10¹⁰ particles/ml (115.1%±26.0%, p<0.01; 139.1%±21.9%, p<0.01; 90.2%±24.4%, p<0.05; and 102.8%±9.0%, p<0.01, for the different growth factors respectively).

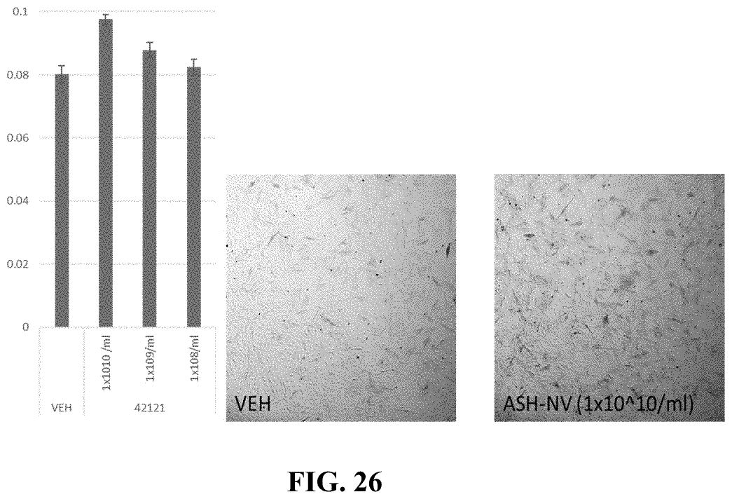

### Example 18. Induction of Melanogenesis in Human Primary Melanocytes (See FIG. 26)

Methods

Human primary melanocytes at passage 5, were seeded in triplicate in 96 well plate and treated with Ashwagandha nanovesicles at 1×10¹⁰, 1×10⁹ and 1×10⁸ particles/mL for 48 hours. The absorbance was read on a plate reader at 400 nm and statistical significance was determine by Students T-test.

### Results

Ashwagandha nanovesicles (ASH-NV) at different concentrations (1×10⁸ NV/mL, 1×10⁹ NV/mL, and 1×10¹⁰ NV/mL), increased melanin production in human primary melanocytes. The effect was dose-dependent and significant at 1×10¹⁰ ASH-NV/mL (Student's t test, p<0.05 vs VEH control).

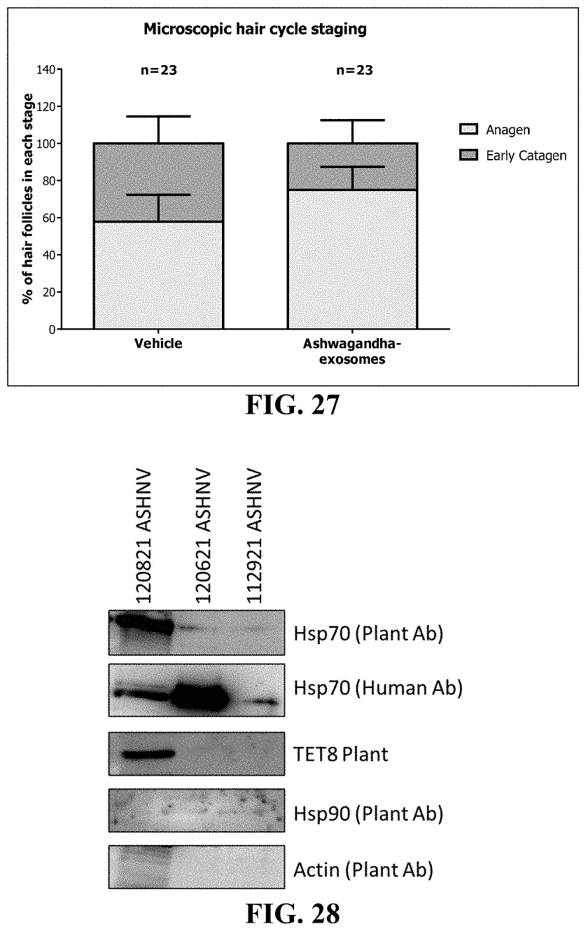

### Example 19: Ashwagandha Nanovesicles Prolong Anagen (Growth) Phase in Ex Vivo Hair Follicles (See FIG. 27)

### Methods

Human micro-dissected hair follicle ex vivo organ culture was used to study the effects of Ashwagandha-nanovesicles (exosomes) over a culture period of 5 days. Human follicles came from 3 donors for 3 different experiments. Exosomes were systemically applied at a concentration of 1×10⁹ nanovesicles/mL and hair growth was assessed by hair cycle staging.

### Results

Treatment with Ashwagandha NV (exosomes) prolongs Anagen Phase after 5 days of culture.

What is claimed is:

1. A composition comprising (a) exosome-like nanovesicles or exosomes and (b) a carrier, wherein the exosome-like nanovesicles or exosomes are extracted from Withania somnifera.

2. The composition of claim 1, wherein the composition is useful for stimulating hair growth or preventing hair loss in a subject.

3. The composition according to any one of the preceding claims, wherein the Withania somnifera is Withania somnifera stem, Withania somnifera root, Withania somnifera leaf, Withania somnifera fruit, or Withania somnifera seed.

4. The composition according to any one of the preceding claims, wherein the exosome-like nanovesicles or exosomes are extracted from Withania somnifera seed.

5. The composition according to any one of the preceding claims, wherein the Withania somnifera is heat-shocked Withania somnifera.

6. The composition according to any one of the preceding claims, wherein the Withania somnifera is not heat shocked.

7. The composition according to any one of the preceding claims, wherein the composition is useful for treating, preventing, or reversing sparse hair growth, short hair growth, thin hair growth, partial or complete hair loss on the scalp, alopecia, androgenic alopecia, alopecia androgenetica, male pattern baldness, female pattern baldness, non-androgenic alopecia, alopecia areata, alopecia totalis, alopecia universalis, radiation induced alopecia, alopecia due to radiotherapy, drug induced alopecia, alopecia due to chemotherapy, traumatic alopecia, scarring alopecia, psychogenic alopecia, stress related alopecia, cortisol related alopecia or anagen effluvium.

8. The composition according to any one of the preceding claims, wherein the composition is a topical composition.

9. The composition according to any one of the preceding claims, wherein the composition is a liquid, an ointment or a cream.

10. The composition according to any one of the preceding claims, wherein the composition is a cosmetic composition.

11. The composition according to any one of the preceding claims, wherein the composition is useful for preventing or reversing cortisol-induced growth arrest in human follicle dermal papilla cells.

12. The composition according to any one of the preceding claims, wherein the Withania somnifera is dried Withania somnifera.

13. The composition according to any one of the preceding claims, wherein the Withania somnifera is dried Withania somnifera seeds.

14. The composition according to any one of the preceding claims, wherein the Withania somnifera is freeze-dried Withania somnifera.

15. The composition according to any one of the preceding claims, wherein the Withania somnifera is freeze-dried Withania somnifera seeds.

16. The composition according to any one of the preceding claims, further comprising aloe-extracted exosome-like nanovesicles or aloe-extracted exosomes.

17. The composition according to any one of the preceding claims, further comprising human exosomes.

18. The composition according to any one of the preceding claims, wherein the number of Withania somnifera-extracted exosome-like nanovesicles or exosomes is from about 1×10⁷ per mL of the composition to about 1×10¹² per mL of the composition.

19. The composition according to claim 18, wherein the number of extracted Withania somnifera-extracted exosome-like nanovesicles or exosomes is about 1×10⁷ per mL of the composition, about 1×10⁸ per mL of the composition, about 1×10⁹ per mL of the composition, about 1×10¹⁰ per mL of the composition, about 1×10¹¹ per mL of the composition, or about 1×10¹² per mL of the composition.

20. The composition according to any one of claims 1-18, wherein the number of Withania somnifera-extracted exosome-like nanovesicles or exosomes is from about 1×10⁹ per mL of the composition to about 1×10¹⁰ per mL of the composition.

21. The composition according to any one of the preceding claims, wherein the number of Withania somnifera-extracted exosome-like nanovesicles or exosomes is about 1×10⁹ per mL of the composition.

22. The composition according to any one of claims 1-20, wherein the number of Withania somnifera-extracted exosome-like nanovesicles or exosomes is about 1×10¹⁰ per mL of the composition.

23. The composition according to any one of the preceding claims, wherein the composition further comprises aloe-extracted exosome-like nanovesicles or exosomes, and the number of the aloe-extracted exosome-like nanovesicles or exosomes is from about 1×10⁷ per mL of the composition to about 1×10¹² per mL of the composition.

24. The composition according to claim 23, wherein the number of aloe-extracted exosome-like nanovesicles or exosomes is about 1×10⁷ per mL, about 1×10⁸ per mL, about 1×10⁹ per mL, about 1×10¹⁰ per mL, about 1×10¹¹ per mL, or about 1×10¹² per mL of the composition.

25. The composition according to claim 23, wherein the number of aloe-extracted exosome-like nanovesicles or exosomes is from about 1×10⁹ per mL to about 1×10¹⁰ per mL of the composition.

26. The composition according to claim 23, wherein the number of aloe-extracted exosome-like nanovesicles or exosomes is about 1×10⁹ per mL of the composition.

27. The composition according to claim 23, wherein the number of aloe-extracted exosome-like nanovesicles or exosomes within the composition is about 1×10¹⁰ per mL of the composition.

28. The composition according to any one of the preceding claims, wherein the Withania somnifera-extracted exosome-like nanovesicles or exosomes are purified.

29. The composition according to any one of the preceding claims, wherein the carrier comprises an aqueous solution, suspension or mixture.

30. The composition according to any one of the preceding claims, wherein the composition further comprises glycerin, Melaleuca alternifolia leaf water, propanediol, 1,2-hexanediol, panthenol, niacinamide, hydroxyethylcellulose, Lepidium meyenii root extract, maltodextrin, caprylhydroxamic acid, Hippophae rhamnoides fruit extract, Equisetum arvense extract, Laminaria saccharina extract, Chondrus crispus extract, sodium metabisulfite, alcohol, phospholipids, arginine, lactic acid, melatonin, potassium sorbate, lactobacillus ferment, Pisum sativum extract, or phosphate buffered saline.

31. The composition according to any one of the preceding claims, wherein the composition further comprises glycerin, Camellia sinensis (green tea) leaf extract, glycine, Larix europaea wood extract, sodium metabisulfite, zinc chloride, Pisum sativum (pea) sprout extract, alcohol, Olea europaea (olive) leaf extract, Curcuma longa(turmeric) root extract, Equisetum arvense (horsetail) extract, Hippophae rhamnoides (sea buckthorn) fruit oil, Laminaria saccharina (neptune kelp) extract, Lepidium meyenii (maca) root extract, Melaleuca alternifolia (tea tree) leaf oil, Moringa oleifera (moringa) leaf extract, Panax ginseng (ginseng) root extract, DL-panthenol, L-theanine, Melatonin, Niacinamide, sodium dehydroacetate, sodium hyaluronate, or phytic acid.

32. The composition according to any one of the preceding claims, wherein the composition further comprises water, glycerin, Melaleuca alternifolia leaf water, propanediol, butylene glycol, caffeine, 1,2-hexanediol, niacinamide, hydroxyethylcellulose, panthenol, Lepidium meyenii root extract, maltodextrin, caprylhydroxamic acid, Chondrus crispus extract, Hippophae rhamnoides fruit extract, Laminaria saccharina (neptune kelp) extract, an alcohol, phospholipids, sodium metabisulfite, arginine, lactic acid, melatonin, potassium sorbate, lactobacillus ferment, Pisum sativum extract, phosphate buffered saline, or Panax ginseng root extract.

33. The composition according to any one of the preceding claims, wherein the carrier is water, and the composition further comprises glycerin, an aqueous buffer, or a naturally occurring preservative.

34. The composition according to any one of the preceding claims, wherein the composition further comprises a naturally occurring preservative.

35. The composition according to claim 34, wherein the naturally occurring preservative comprises lactobacillus ferment.

36. The composition according to any one of the preceding claims, wherein the composition further comprises melatonin.

37. The composition according to any one of the preceding claims, wherein the composition further comprises niacinamide.

38. The composition according to any one of the preceding claims, wherein the composition further comprises an alcohol.

39. The composition according to claim 38, wherein the alcohol is ethyl alcohol.

40. The composition according to any one of the preceding claims, wherein the composition comprises the Withania somnifera-extracted exosome-like nanovesicles or exosomes in an amount of about 0.01% to 10% by weight of the composition.

41. The composition according to claim 40, wherein the composition comprises the Withania somnifera-extracted exosome-like nanovesicles or exosomes in an amount of about 0.1% to 5% by weight of the composition.

42. The composition according to claim 41, wherein the composition comprises the Withania somnifera-extracted exosome-like nanovesicles or exosomes in an amount of about 0.1% to 4% by weight of the composition.

43. The composition according to claim 42, wherein the composition comprises the Withania somnifera-extracted exosome-like nanovesicles or exosomes in an amount of about 0.1% to 3% by weight of the composition.

44. The composition according to claim 43, wherein the composition comprises the Withania somnifera-extracted exosome-like nanovesicles or exosomes in an amount of about 0.1% to 2% by weight of the composition.

45. The composition according to claim 44, wherein the composition comprises the Withania somnifera-extracted exosome-like nanovesicles or exosomes in an amount of about 0.1% to 1% by weight of the composition.

46. The composition according to claim 45, wherein the composition comprises the Withania somnifera-extracted exosome-like nanovesicles or exosomes in an amount of about 0.3% to about 1% by weight of the composition.

47. A method of promoting hair growth or reducing hair loss, comprising administering to a subject in need thereof an effective amount of the composition of any one of claims 1-46 for a time sufficient to promote hair growth or reduce hair loss.

48. A method of preventing, reducing or reversing hair loss, comprising administering to a subject in need thereof an effective amount of a composition according to any one of claims 1-46 for a time sufficient to prevent, reduce or reverse hair loss.

49. The method of claim 47 or 48, wherein the hair loss is caused or mediated by cortisol or stress.

50. A method for effecting a change in mammalian hair appearance, hair growth, hair pigmentation, hair follicle size or hair shaft size, comprising administering to a subject in need thereof an effective amount of a composition according to any one of claims 1-46 for a time sufficient to effect a change in mammalian hair appearance, hair growth, hair pigmentation, hair follicle size or hair shaft size.

51. The method of claim 50, wherein the administering is topically administering.

52. A method for producing a melanogenetic action in hair or promoting its pigmentation, comprising administering to a subject in need thereof an effective amount of a composition according to any one of claims 1-46 for a time sufficient to produce a melanogenetic action in the hair or promote its pigmentation.

53. A method of stimulating hair growth or preventing hair loss, comprising topically administering to a subject in need thereof a composition comprising (a) Withania somnifera-extracted exosome-like nanovesicles or exosomes and (b) a carrier, wherein:
the amount of the Withania somnifera-extracted exosome-like nanovesicles or exosomes is from about 0.1% to about 5% by weight of the composition,
the number of Withania somnifera-extracted exosome-like nanovesicles or exosomes is from about 1×10⁸ per mL of the composition to about 1×10¹⁰ per mL of the composition, and the Withania somnifera is dried Withania somnifera seeds.

54. A method of promoting hair growth or reducing hair loss, comprising
administering to dermal papilla of a subject in need thereof a composition comprising Withania somnifera-extracted exosome-like nanovesicles or exosomes having an increased level of heat shock stress-response exosomes,
wherein the Withania somnifera-extracted exosome-like nanovesicles or exosomes are extracted from Withania somnifera stem, Withania somnifera root, Withania somnifera leaf, or Withania somnifera fruit of a Withania somnifera plant, wherein the Withania somnifera plant is grown at a conditioning temperature.

55. The method according to claim 54, wherein the conditioning temperature is about 33° C. to about 45° C.

56. The method according to claim 54 or 55, wherein the Withania somnifera plant is grown at the conditioning temperature for about 1 hour to about 5 hours.

57. The method according to claim 56, wherein the Withania somnifera plant is grown at a conditioning temperature of about 33° C. to about 45° C. for about 1 hour to about 5 hours.

58. The method according to any one of claims 54-57, wherein the conditioning temperature is about 45° C.

59. The method according to any one of claims 54-58, wherein the Withania somnifera plant is primed by warming it at a priming temperature prior to growing it at the conditioning temperature.

60. The method of claim 59, wherein the priming temperature is about 20° C. to about 33° C.

61. A kit for promoting hair growth or preventing, reducing, or reversing hair loss, comprising the composition of any one of claims 1-46 and instructions for topically administering the composition to a scalp of a subject in need of hair-growth promotion or hair-loss prevention, reduction or reversal.

62. Use of a composition according to any one of claims 1-46 for promoting hair growth or preventing, reducing, or reversing hair loss in a subject in need thereof.
